SEQUENCE LISTING (144 sequences)

SEQ ID NO: 1 - DNA, 10576 bp, Artificial sequence
  Synthetic sequence pEAQ-HT-CPMV/24K
cctgtggttggcatgcacatacaaatggacgaacggataaaccttttcacgcccttttaaatatccgattattctaataaacgctcttttctcttaggtttacccgccaatatatcctgtcaaacactgatagtttgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgttaattaagaattcgagctccaccgcggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaattcgcgatgtctttggatcagagtagtgttgctatcatgtctaagtgtagggctaatctggtttttggaggcactaatttgcaaatagtcatggtaccaggaagacgctttttggcatgcaaacatttcttcacccacataaagaccaaattgcgtgtggaaatagttatggatggaagaaggtactatcatcaatttgatcctgcaaatatttatgatatacctgattctgagttggtcttgtactcccatcctagcttggaagacgtttcccattcttgctgggatctgttctgttgggacccagacaaagaattgccttcagtatttggagcggatttcttgagttgtaaatacaacaagtttgggggtttttatgaggcgcaatatgctgacatcaaagtgcgcacaaagaaagaatgccttaccatacagagtggtaattatgtgaacaaggtgtctcgctatcttgagtatgaagctcctactatccctgaggattgtggatctcttgtgatagcacacattggtgggaagcacaagattgtgggtgttcatgttgctggtattcaaggtaagataggatgtgcttccttattgccaccattggagccaatagcacaagcgcaatagctcgaggcctttaactctggtttcattaaattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgccagcttggcgtaatcatggtcatagctgttgcgattaagaattcgagctcggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttcgactctagaggatccccttaaatcgatatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtctcgaactcttcagcatctctgtgagatggcaattcggtctaagcaagaactgctacagcttgccccaatcgaagtggaaagtaatgtatcaagaggatgccctgaaggtactgagaccttcgaaaaagaaagcgagtaaggggagctcgaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatggcgcgtggccggcctacagtatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgcggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactgtaggccggccctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagagtagacaggctgtctcgcggccgaggggcgcagcccctgggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacactagtcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaactatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcaggtaacctcgcgcatacagccgggcagtgacgtcatcgtctgcgcggaaatggacgggcccccggcgccagatctggggaac

SEQ ID NO: 2 - DNA, 11712 bp, Artificial sequence
  Synthetic sequence pEAQ-HT-CPMV/VP60
cctgtggttggcatgcacatacaaatggacgaacggataaaccttttcacgcccttttaaatatccgattattctaataaacgctcttttctcttaggtttacccgccaatatatcctgtcaaacactgatagtttgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgttaattaagaattcgagctccaccgcggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaattcgcgatggagcaaaacttgtttgccctttctttggatgatacaagctcagttcgtggttctttgcttgacacaaaattcgcacaaactcgagttttgttgtccaaggctatggctggtggtgatgtgttattggatgagtatctctatgatgtggtcaatggacaagattttagagctactgtcgcttttttgcgcacccatgttataacaggcaaaataaaggtgacagctaccaccaacatttctgacaactcgggttgttgtttgatgttggccataaatagtggtgtgaggggtaagtatagtactgatgtttatactatctgctctcaagactccatgacgtggaacccagggtgcaaaaagaacttctcgttcacatttaatccaaacccttgtggggattcttggtctgctgagatgataagtcgaagcagagttaggatgacagttatttgtgtttcgggatggaccttatctcctaccacagatgtgattgccaagctagactggtcaattgtcaatgagaaatgtgagcccaccatttaccacttggctgattgtcagaattggttaccccttaatcgttggatgggaaaattgacttttccccagggtgtgacaagtgaggttcgaaggatgcctctttctataggaggcggtgctggtgcgactcaagctttcttggccaatatgcccaattcatggatatcaatgtggagatattttagaggtgaacttcactttgaagttactaaaatgagctctccatatattaaagccactgttacatttctcatagcttttggtaatcttagtgatgcctttggtttttatgagagttttcctcatagaattgttcaatttgctgaggttgaggaaaaatgtactttggttttctcccaacaagagtttgtcactgcttggtcaacacaagtaaaccccagaaccacacttgaagcagatggttgtccctacctatatgcaattattcatgatagtacaacaggtacaatctccggagattttaatcttggggtcaagcttgttggcattaaggatttttgtggtataggttctaatccgggtattgatggttcccgcttgcttggagctatagcacaaggacctgtttgtgctgaagcctcagatgtgtatagcccatgtatgatagctagcactcctcctgctccattttcagacgtcacagcagtaacttttgacttaatcaacggcaaaataactcctgttggtgatgacaattggaatacgcacatttataatcctccaattatgaatgtcttgcgtactgctgcttggaaatctggaactattcatgttcaacttaatgttaggggtgctggtgtcaaaagagcagattgggatggtcaagtctttgtttacctgcgccagtccatgaaccctgaaagttatgatgcgcggacatttgtgatctcacaacctggttctgccatgttgaacttctcttttgatatcatagggccgaatagcggatttgaatttgccgaaagcccatgggccaatcagaccacctggtatcttgaatgtgttgctaccaatcccagacaaatacagcaatttgaggtcaacatgcgcttcgatcctaatttcagggttgccggcaatatcctgatgcccccatttccactgtcaacggaaactccaccgttattaaagtttaggtttcgggatattgaacgctccaagcgtagtgttatggttggacacactgctactgctgcttagtcgaggcctttaactctggtttcattaaattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgccagcttggcgtaatcatggtcatagctgttgcgattaagaattcgagctcggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttcgactctagaggatccccttaaatcgatatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtctcgaactcttcagcatctctgtgagatggcaattcggtctaagcaagaactgctacagcttgccccaatcgaagtggaaagtaatgtatcaagaggatgccctgaaggtactgagaccttcgaaaaagaaagcgagtaaggggagctcgaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatggcgcgtggccggcctacagtatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgcggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactgtaggccggccctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagagtagacaggctgtctcgcggccgaggggcgcagcccctgggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacactagtcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaactatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcaggtaacctcgcgcatacagccgggcagtgacgtcatcgtctgcgcggaaatggacgggcccccggcgccagatctggggaac

SEQ ID NO: 3 - DNA, 11937 bp, Artificial sequence
  Synthetic sequence pEAQexpress VP60-24K
taagaattcgagctccaccgcggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaattcgcgatggagcaaaacttgtttgccctttctttggatgatacaagctcagttcgtggttctttgcttgacacaaaattcgcacaaactcgagttttgttgtccaaggctatggctggtggtgatgtgttattggatgagtatctctatgatgtggtcaatggacaagattttagagctactgtcgcttttttgcgcacccatgttataacaggcaaaataaaggtgacagctaccaccaacatttctgacaactcgggttgttgtttgatgttggccataaatagtggtgtgaggggtaagtatagtactgatgtttatactatctgctctcaagactccatgacgtggaacccagggtgcaaaaagaacttctcgttcacatttaatccaaacccttgtggggattcttggtctgctgagatgataagtcgaagcagagttaggatgacagttatttgtgtttcgggatggaccttatctcctaccacagatgtgattgccaagctagactggtcaattgtcaatgagaaatgtgagcccaccatttaccacttggctgattgtcagaattggttaccccttaatcgttggatgggaaaattgacttttccccagggtgtgacaagtgaggttcgaaggatgcctctttctataggaggcggtgctggtgcgactcaagctttcttggccaatatgcccaattcatggatatcaatgtggagatattttagaggtgaacttcactttgaagttactaaaatgagctctccatatattaaagccactgttacatttctcatagcttttggtaatcttagtgatgcctttggtttttatgagagttttcctcatagaattgttcaatttgctgaggttgaggaaaaatgtactttggttttctcccaacaagagtttgtcactgcttggtcaacacaagtaaaccccagaaccacacttgaagcagatggttgtccctacctatatgcaattattcatgatagtacaacaggtacaatctccggagattttaatcttggggtcaagcttgttggcattaaggatttttgtggtataggttctaatccgggtattgatggttcccgcttgcttggagctatagcacaaggacctgtttgtgctgaagcctcagatgtgtatagcccatgtatgatagctagcactcctcctgctccattttcagacgtcacagcagtaacttttgacttaatcaacggcaaaataactcctgttggtgatgacaattggaatacgcacatttataatcctccaattatgaatgtcttgcgtactgctgcttggaaatctggaactattcatgttcaacttaatgttaggggtgctggtgtcaaaagagcagattgggatggtcaagtctttgtttacctgcgccagtccatgaaccctgaaagttatgatgcgcggacatttgtgatctcacaacctggttctgccatgttgaacttctcttttgatatcatagggccgaatagcggatttgaatttgccgaaagcccatgggccaatcagaccacctggtatcttgaatgtgttgctaccaatcccagacaaatacagcaatttgaggtcaacatgcgcttcgatcctaatttcagggttgccggcaatatcctgatgcccccatttccactgtcaacggaaactccaccgttattaaagtttaggtttcgggatattgaacgctccaagcgtagtgttatggttggacacactgctactgctgcttagtcgaggcctttaactctggtttcattaaattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgccagcttggcgtaatcatggtcatagctgttgcgattaagaattcgagctccaccgcggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaattcgcgatgtctttggatcagagtagtgttgctatcatgtctaagtgtagggctaatctggtttttggaggcactaatttgcaaatagtcatggtaccaggaagacgctttttggcatgcaaacatttcttcacccacataaagaccaaattgcgtgtggaaatagttatggatggaagaaggtactatcatcaatttgatcctgcaaatatttatgatatacctgattctgagttggtcttgtactcccatcctagcttggaagacgtttcccattcttgctgggatctgttctgttgggacccagacaaagaattgccttcagtatttggagcggatttcttgagttgtaaatacaacaagtttgggggtttttatgaggcgcaatatgctgacatcaaagtgcgcacaaagaaagaatgccttaccatacagagtggtaattatgtgaacaaggtgtctcgctatcttgagtatgaagctcctactatccctgaggattgtggatctcttgtgatagcacacattggtgggaagcacaagattgtgggtgttcatgttgctggtattcaaggtaagataggatgtgcttccttattgccaccattggagccaatagcacaagcgcaatagctcgaggcctttaactctggtttcattaaattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgtggccggccatcttttatctttagagttaagaactctttcgtattttggtgaggttttatcctcttgagttttggtcatagacctattcatggctctgataccaatttttaagcgggggcttatgcggattatttcttaaattgataaggggttattagggggtatagggtataaatacaagcattcccttagcgtatagtataagtatagtagcgtacctctatcaaatttccatcttcttaccttgcacagggcctgcaaccttatccttccttgtcttcctccttccttccgtccacttcatcatatttaaaccaaacctacgggggagtcaacgtaaccaaccctgccttagcatcttttccctaacggcctcctgcctaagcggtacttctagcttcgaacggcgtctgggctccaggtttagtcgtctcgtgtctggtttatattcacgacaaagatctatagggactttaggagatctggattttagtactggattttggttttaggaattagaaattttattgatagaagtattttacaaatacaaatacatactaagggtttcttatatgctcaacacatgagcgaaaccctataagaaccctaatttcccttatcgggaaactactcacacattatttatggagaaaatagagagagatagatttgtagagagagactggtgatttcagcgaattcgagctccccttactcgctttctttttcgaaggtctcagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagtggtacctcctgatcctccatcccaacgttcactgttagcttgttccctagcgtcgtttccttgtatagctcgttccatatcgatttaaggggatcctctagagtcgaagcttgggctgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattaccctttgttgaaaagtctcaatagccctttggtcttctgagactgtatctttgacatttttggagtaggggggtaccgagctcgaattcggccggccctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagagtagacaggctgtctcgcggccgaggggcgcagcccctgggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacactagtcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaactatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcaggtaacctcgcgcatacagccgggcagtgacgtcatcgtctgcgcggaaatggacgggcccccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgttaat

SEQ ID NO: 4 - DNA, 3481 bp, Cowpea mosaic virus
tattaaaatcttaataggttttgataaaagcgaacgtggggaaacccgaaccaaaccttcttctaaattctctctcatctctcttaaagcaaacttctctcttgtctttcttgcatgagcgatcttcaacgttgtcagatcgtgcttcggcaccagtacaatgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgaaatggaaagcattatgagccgtggtattccttcaggaattttggaggaaaaagctattcagttcaaacgtgccaaagaagggaataaacccttgaaggatgagattcccaagcctgaggatatgtatgtgtctcacacttctaaatggaatgtgctcagaaaaatgagccaaaagactgtggatctttccaaagcagctgctgggatgggattcatcaataagcatatgcttacgggcaacatcttggcacaaccaacaacagtcttggatattcccgtcacaaaggataaaacacttgcgatggccagtgattttattcgtaaggagaatctcaagacttctgccattcacattggagcaattgagattattatccagagctttgcttcccctgaaagtgatttgatgggaggctttttgcttgtggattctttacacactgatacagctaatgctattcgtagcatttttgttgctccaatgcggggaggaagaccagtcagagtggtgaccttcccaaatacactggcacctgtatcatgtgatctgaacaatagattcaagctcatttgctcattgccaaactgtgatattgtccagggtagccaagtagcagaagtgagtgtaaatgttgcaggatgtgctacttccatagagaaatctcacaccccttcccaattgtatacagaggaatttgaaaaggagggtgctgttgttgtagaatacttaggcagacagacctattgtgctcagcctagcaatttacccacagaagaaaaacttcggtcccttaagtttgactttcatgttgaacaaccaagtgtcctgaagttatccaattcctgcaatgcgcactttgtcaagggagaaagtttgaaatactctatttctggcaaagaagcagaaaaccatgcagttcatgctactgtggtctctcgagaaggggcttctgcggcacccaagcaatatgatcctattttgggacgggtgctggatccacgaaatgggaatgtggcttttccacaaatggagcaaaacttgtttgccctttctttggatgatacaagctcagttcgtggttctttgcttgacacaaaattcgcacaaactcgagttttgttgtccaaggctatggctggtggtgatgtgttattggatgagtatctctatgatgtggtcaatggacaagattttagagctactgtcgcttttttgcgcacccatgttataacaggcaaaataaaggtgacagctaccaccaacatttctgacaactcgggttgttgtttgatgttggccataaatagtggtgtgaggggtaagtatagtactgatgtttatactatctgctctcaagactccatgacgtggaacccagggtgcaaaaagaacttctcgttcacatttaatccaaacccttgtggggattcttggtctgctgagatgataagtcgaagcagagttaggatgacagttatttgtgtttcgggatggaccttatctcctaccacagatgtgattgccaagctagactggtcaattgtcaatgagaaatgtgagcccaccatttaccacttggctgattgtcagaattggttaccccttaatcgttggatgggaaaattgacttttccccagggtgtgacaagtgaggttcgaaggatgcctctttctataggaggcggtgctggtgcgactcaagctttcttggccaatatgcccaattcatggatatcaatgtggagatattttagaggtgaacttcactttgaagttactaaaatgagctctccatatattaaagccactgttacatttctcatagcttttggtaatcttagtgatgcctttggtttttatgagagttttcctcatagaattgttcaatttgctgaggttgaggaaaaatgtactttggttttctcccaacaagagtttgtcactgcttggtcaacacaagtaaaccccagaaccacacttgaagcagatggttgtccctacctatatgcaattattcatgatagtacaacaggtacaatctccggagattttaatcttggggtcaagcttgttggcattaaggatttttgtggtataggttctaatccgggtattgatggttcccgcttgcttggagctatagcacaaggacctgtttgtgctgaagcctcagatgtgtatagcccatgtatgatagctagcactcctcctgctccattttcagacgttacagcagtaacttttgacttaatcaacggcaaaataactcctgttggtgatgacaattggaatacgcacatttataatcctccaattatgaatgtcttgcgtactgctgcttggaaatctggaactattcatgttcaacttaatgttaggggtgctggtgtcaaaagagcagattgggatggtcaagtctttgtttacctgcgccagtccatgaaccctgaaagttatgatgcgcggacatttgtgatctcacaacctggttctgccatgttgaacttctcttttgatatcatagggccgaatagcggatttgaatttgccgaaagcccatgggccaatcagaccacctggtatcttgaatgtgttgctaccaatcccagacaaatacagcaatttgaggtcaacatgcgcttcgatcctaatttcagggttgccggcaatatcctgatgcccccatttccactgtcaacggaaactccaccgttattaaagtttaggtttcgggatattgaacgctccaagcgtagtgttatggttggacacactgctactgctgcttaactctggtttcattaaattttctttagtttgaatttactgttatttggtgtgcatttctatgtttggtgagcggttttctgtgctcagagtgtgtttattttatgtaatttaatttctttgtgagctcctgtttagcaggtcgtcccttcagcaaggacacaaaaagattttaattttatt

SEQ ID NO: 5 - DNA, 31 bp, Artificial sequence
  Synthetic sequence Oligonucleotide A115G-F
cttgtctttcttgcgtgagcgatcttcaacg

SEQ ID NO: 6 - DNA, 31 bp, Artificial sequence
  Synthetic sequence Oligonucleotide A115G-R
cgttgaagatcgctcacgcaagaaagacaag

SEQ ID NO: 7 - DNA, 33 bp, Artificial sequence
  Synthetic sequence Oligonucleotide U162C-F
ggcaccagtacaacgttttctttcactgaagcg

SEQ ID NO: 8 - DNA, 33 bp, Artificial sequence
  Synthetic sequence Oligonucleotide U162C-R
cgcttcagtgaaagaaaacgttgtactggtgcc

SEQ ID NO: 9 - DNA, 35 bp, Artificial sequence
  Synthetic sequence Oligonucleotide KS 19
gagtttgggcagatctagaaatgtctttggatcag

SEQ ID NO: 10 - DNA, 60 bp, Cowpea mosaic virus
ggtacaacaatgttcctctcaagagaagagtttgggcagacgcacaaatgtctttggatc

SEQ ID NO: 11 - protein, 20 aa, Cowpea mosaic virus
GTTMFLSREEFGQTHKCLWI

SEQ ID NO: 12 - protein, 20 aa, Cowpea mosaic virus
VQQCSSQEKSLGRRTNVFGS

SEQ ID NO: 13 - protein, 20 aa, Cowpea mosaic virus
YNNVPLKRRVWADAQMSLDQ

SEQ ID NO: 14 - DNA, 59 bp, Cowpea mosaic virus
gatccaaagacatttgtgcgtctgcccaaactcttctcttgagaggaacattgttgtac

SEQ ID NO: 15 - DNA, 34 bp, Artificial sequence
  Synthetic sequence Oligonucleotide KS 20
cttcggactagtctattgcgcttgtgctattggc

SEQ ID NO: 16 - DNA, 60 bp, Cowpea mosaic virus
cacaagcgcaaggtgctgaggaatactttgattttcttccagctgaagagaatgtatctt

SEQ ID NO: 17 - protein, 20 aa, Cowpea mosaic virus
HKRKVLRNTLIFFQLKRMYL

SEQ ID NO: 18 - protein, 5 aa, Cowpea mosaic virus
TSARC

SEQ ID NO: 19 - protein, 4 aa, Cowpea mosaic virus
FSSS

SEQ ID NO: 20 - protein, 5 aa, Cowpea mosaic virus
RECIF

SEQ ID NO: 21 - protein, 20 aa, Cowpea mosaic virus
QAQGAEEYFDFLPAEENVSS

SEQ ID NO: 22 - DNA, 60 bp, Cowpea mosaic virus
aagatacattctcttcagctggaagaaaatcaaagtattcctcagcaccttgcgcttgtg

SEQ ID NO: 23 - DNA, 32 bp, Artificial sequence
  Synthetic sequence Oligonucleotide KS 17
ggctagtgatcacacaaatggagcaaaacttg

SEQ ID NO: 24 - DNA, 60 bp, Cowpea mosaic virus
gctggatccacgaaatgggaatgtggcttttccacaaatggagcaaaacttgtttgccct

SEQ ID NO: 25 - protein, 20 aa, Cowpea mosaic virus
AGSTKWECGFSTNGAKLVCP

SEQ ID NO: 26 - protein, 20 aa, Cowpea mosaic virus
LDPRNGNVAFPQMEQNLFAL

SEQ ID NO: 27 - protein, 20 aa, Cowpea mosaic virus
WIHEMGMWLFHKWSKTCLPF

SEQ ID NO: 28 - DNA, 60 bp, Cowpea mosaic virus
agggcaaacaagttttgctccatttgtggaaaagccacattcccatttcgtggatccagc

SEQ ID NO: 29 - DNA, 31 bp, Artificial sequence
  Synthetic sequence Oligonucleotide KS 18
taatgaattcccagagttaagcagcagtagc

SEQ ID NO: 30 - DNA, 120 bp, Cowpea mosaic virus
tcgggatattgaacgctccaagcgtagtgttatggttggacacactgctactgctgcttaactctggtttcattaaattttctttagtttgaatttactgttatttggtgtgcatttcta

SEQ ID NO: 31 - protein, 4 aa, Cowpea mosaic virus
TLQA

SEQ ID NO: 32 - protein, 11 aa, Cowpea mosaic virus
CYGWTHCYCCL

SEQ ID NO: 33 - protein, 19 aa, Cowpea mosaic virus
RDIERSKRSVMVGHTATAA

SEQ ID NO: 34 - protein, 20 aa, Cowpea mosaic virus
GILNAPSVVLWLDTLLLLLN

SEQ ID NO: 35 - DNA, 120 bp, Cowpea mosaic virus
tagaaatgcacaccaaataacagtaaattcaaactaaagaaaatttaatgaaaccagagttaagcagcagtagcagtgtgtccaaccataacactacgcttggagcgttcaatatcccga

SEQ ID NO: 36 - DNA, 26 bp, Artificial sequence
  Synthetic sequence Oligonucleotide KS11
gtcggatcccaacatgggtctcccag

SEQ ID NO: 37 - DNA, 60 bp, Cowpea mosaic virus
cgggactttcttagtcttgacccaacatgggtctcccagaatatgaggccgatagtgagg

SEQ ID NO: 38 - protein, 20 aa, Cowpea mosaic virus
RDFLSLDPTWVSQNMRPIVR

SEQ ID NO: 39 - protein, 14 aa, Cowpea mosaic virus
GTFLVLTQHGSPRI

SEQ ID NO: 40 - protein, 14 aa, Cowpea mosaic virus
PNMGLPEYEADSEA

SEQ ID NO: 41 - DNA, 60 bp, Cowpea mosaic virus
cctcactatcggcctcatattctgggagacccatgttgggtcaagactaagaaagtcccg

SEQ ID NO: 42 - DNA, 20 bp, Artificial sequence
  Synthetic sequence Oligonucleotide KS 10
ttatcctagtttgcgcgcta

SEQ ID NO: 43 - DNA, 624 bp, Cowpea mosaic virus
atgtctttggatcagagtagtgttgctatcatgtctaagtgtagggctaatctggtttttggaggcactaatttgcaaatagtcatggtaccaggaagacgctttttggcatgcaaacatttcttcacccacataaagaccaaattgcgtgtggaaatagttatggatggaagaaggtactatcatcaatttgatcctgcaaatatttatgatatacctgattctgagttggtcttgtactcccatcctagcttggaagacgtttcccattcttgctgggatctgttctgttgggacccagacaaagaattgccttcagtatttggagcggatttcttgagttgtaaatacaacaagtttgggggtttttatgaggcgcaatatgctgatatcaaagtgcgcacaaagaaagaatgccttaccatacagagtggtaattatgtgaacaaggtgtctcgctatcttgagtatgaagctcctactatccctgaggattgtggatctcttgtgatagcacacattggtgggaagcacaagattgtgggtgttcatgttgctggtattcaaggtaagataggatgtgcttccttattgccaccattggagccaatagcacaagcgcaa

SEQ ID NO: 44 - protein, 26 aa, Cowpea mosaic virus
CLWIRVVLLSCLSVGLIWFLEALICK

SEQ ID NO: 45 - protein, 17 aa, Cowpea mosaic virus
SWYQEDAFWHANISSPT

SEQ ID NO: 46 - protein, 7 aa, Cowpea mosaic virus
RPNCVWK

SEQ ID NO: 47 - protein, 59 aa, Cowpea mosaic virus
LWMEEGTIINLILQIFMIYLILSWSCTPILAWKTFPILAGICSVGTQTKNCLQYLERIS

SEQ ID NO: 48 - protein, 34 aa, Cowpea mosaic virus
VVNTTSLGVFMRRNMLISKCAQRKNALPYRVVIM

SEQ ID NO: 49 - protein, 21 aa, Cowpea mosaic virus
TRCLAILSMKLLLSLRIVDLL

SEQ ID NO: 50 - protein, 20 aa, Cowpea mosaic virus
HTLVGSTRLWVFMLLVFKVR

SEQ ID NO: 51 - protein, 11 aa, Cowpea mosaic virus
DVLPYCHHWSQ

SEQ ID NO: 52 - protein, 5 aa, Cowpea mosaic virus
HKRKV

SEQ ID NO: 53 - protein, 5 aa, Cowpea mosaic virus
VFGSE

SEQ ID NO: 54 - protein, 5 aa, Cowpea mosaic virus
CCYHV

SEQ ID NO: 55 - protein, 6 aa, Cowpea mosaic virus
SGFWRH

SEQ ID NO: 56 - protein, 40 aa, Cowpea mosaic virus
FANSHGTRKTLFGMQTFLHPHKDQIACGNSYGWKKVLSSI

SEQ ID NO: 57 - protein, 5 aa, Cowpea mosaic virus
SCKYL

SEQ ID NO: 58 - protein, 7 aa, Cowpea mosaic virus
VGLVLPS

SEQ ID NO: 59 - protein, 31 aa, Cowpea mosaic virus
LGRRFPFLLGSVLLGPRQRIAFSIWSGFLEL

SEQ ID NO: 60 - protein, 8 aa, Cowpea mosaic virus
IQQVWGFL

SEQ ID NO: 61 - protein, 4 aa, Cowpea mosaic virus
GAIC

SEQ ID NO: 62 - protein, 15 aa, Cowpea mosaic virus
YQSAHKERMPYHTEW

SEQ ID NO: 63 - protein, 9 aa, Cowpea mosaic virus
LCEQGVSLS

SEQ ID NO: 64 - protein, 5 aa, Cowpea mosaic virus
SSYYP

SEQ ID NO: 65 - protein, 26 aa, Cowpea mosaic virus
GLWISCDSTHWWEAQDCGCSCCWYSR

SEQ ID NO: 66 - protein, 19 aa, Cowpea mosaic virus
DRMCFLIATIGANSTSARC

SEQ ID NO: 67 - protein, 207 aa, Cowpea mosaic virus
MSLDQSSVAIMSKCRANLVFGGTNLQIVMVPGRRFLACKHFFTHIKTKLRVEIVMDGRRYYHQFDPANIYDIPDSELVLYSHPSLEDVSHSCWDLFCWDPDKELPSVFGADFLSCKYNKFGGFYEAQYADIKVRTKKECLTIQSGNYVNKVSRYLEYEAPTIPEDCGSLVIAHIGGKHKIVGVHVAGIQGKIGCASLLPPLEPIQAQ

SEQ ID NO: 68 - DNA, 624 bp, Cowpea mosaic virus
ttgcgcttgtgctattggctccaatggtggcaataaggaagcacatcctatcttaccttgaataccagcaacatgaacacccacaatcttgtgcttcccaccaatgtgtgctatcacaagagatccacaatcctcagggatagtaggagcttcatactcaagatagcgagacaccttgttcacataattaccactctgtatggtaaggcattctttctttgtgcgcactttgatatcagcatattgcgcctcataaaaacccccaaacttgttgtatttacaactcaagaaatccgctccaaatactgaaggcaattctttgtctgggtcccaacagaacagatcccagcaagaatgggaaacgtcttccaagctaggatgggagtacaagaccaactcagaatcaggtatatcataaatatttgcaggatcaaattgatgatagtaccttcttccatccataactatttccacacgcaatttggtctttatgtgggtgaagaaatgtttgcatgccaaaaagcgtcttcctggtaccatgactatttgcaaattagtgcctccaaaaaccagattagccctacacttagacatgatagcaacactactctgatccaaagacat

SEQ ID NO: 69 - DNA, 1764 bp, Cowpea mosaic virus
atggagcaaaacttgtttgccctttctttggatgatacaagctcagttcgtggttctttgcttgacacaaaattcgcacaaactcgagttttgttgtccaaggctatggctggtggtgatgtgttattggatgagtatctctatgatgtggtcaatggacaagattttagagctactgtcgcttttttgcgcacccatgttataacaggcaaaataaaggtgacagctaccaccaacatttctgacaactcgggttgttgtttgatgttggccataaatagtggtgtgaggggtaagtatagtactgatgtttatactatctgctctcaagactccatgacgtggaacccagggtgcaaaaagaacttctcgttcacatttaatccaaacccttgtggggattcttggtctgctgagatgataagtcgaagcagagttaggatgacagttatttgtgtttcgggatggaccttatctcctaccacagatgtgattgccaagctagactggtcaattgtcaatgagaaatgtgagcccaccatttaccacttggctgattgtcagaattggttaccccttaatcgttggatgggaaaattgacttttccccagggtgtgacaagtgaggttcgaaggatgcctctttctataggaggcggtgctggtgcgactcaagctttcttggccaatatgcccaattcatggatatcaatgtggagatattttagaggtgaacttcactttgaagttactaaaatgagctctccatatattaaagccactgttacatttctcatagcttttggtaatcttagtgatgcctttggtttttatgagagttttcctcatagaattgttcaatttgctgaggttgaggaaaaatgtactttggttttctcccaacaagagtttgtcactgcttggtcaacacaagtaaaccccagaaccacacttgaagcagatggttgtccctacctatatgcaattattcatgatagtacaacaggtacaatctccggagattttaatcttggggtcaagcttgttggcattaaggatttttgtggtataggttctaatccgggtattgatggttcccgcttgcttggagctatagcacaaggacctgtttgtgctgaagcctcagatgtgtatagcccatgtatgatagctagcactcctcctgctccattttcagacgttacagcagtaacttttgacttaatcaacggcaaaataactcctgttggtgatgacaattggaatacgcacatttataatcctccaattatgaatgtcttgcgtactgctgcttggaaatctggaactattcatgttcaacttaatgttaggggtgctggtgtcaaaagagcagattgggatggtcaagtctttgtttacctgcgccagtccatgaaccctgaaagttatgatgcgcggacatttgtgatctcacaacctggttctgccatgttgaacttctcttttgatatcatagggccgaatagcggatttgaatttgccgaaagcccatgggccaatcagaccacctggtatcttgaatgtgttgctaccaatcccagacaaatacagcaatttgaggtcaacatgcgcttcgatcctaatttcagggttgccggcaatatcctgatgcccccatttccactgtcaacggaaactccaccgttattaaagtttaggtttcgggatattgaacgctccaagcgtagtgttatggttggacacactgctactgctgcttaa

SEQ ID NO: 70 - protein, 10 aa, Cowpea mosaic virus
GAKLVCPFFG

SEQ ID NO: 71 - protein, 9 aa, Cowpea mosaic virus
YKLSSWFFA

SEQ ID NO: 72 - protein, 17 aa, Cowpea mosaic virus
HKIRTNSSFVVQGYGWW

SEQ ID NO: 73 - protein, 4 aa, Cowpea mosaic virus
CVIG

SEQ ID NO: 74 - protein, 7 aa, Cowpea mosaic virus
CGQWTRF

SEQ ID NO: 75 - protein, 24 aa, Cowpea mosaic virus
SYCRFFAHPCYNRQNKGDSYHQHF

SEQ ID NO: 76 - protein, 11 aa, Cowpea mosaic virus
QLGLLFDVGHK

SEQ ID NO: 77 - protein, 4 aa, Cowpea mosaic virus
WCEG

SEQ ID NO: 78 - protein, 24 aa, Cowpea mosaic virus
CLYYLLSRLHDVEPRVQKELLVHI

SEQ ID NO: 79 - protein, 10 aa, Cowpea mosaic virus
SKPLWGFLVC

SEQ ID NO: 80 - protein, 7 aa, Cowpea mosaic virus
DDKSKQS

SEQ ID NO: 81 - protein, 27 aa, Cowpea mosaic virus
DDSYLCFGMDLISYHRCDCQARLVNCQ

SEQ ID NO: 82 - protein, 7 aa, Cowpea mosaic virus
AHHLPLG

SEQ ID NO: 83 - protein, 7 aa, Cowpea mosaic virus
LSELVTP

SEQ ID NO: 84 - protein, 14 aa, Cowpea mosaic virus
SLDGKIDFSPGCDK

SEQ ID NO: 85 - protein, 33 aa, Cowpea mosaic virus
GSKDASFYRRRCWCDSSFLGQYAQFMDINVEIF

SEQ ID NO: 86 - protein, 6 aa, Cowpea mosaic virus
NELSIY

SEQ ID NO: 87 - protein, 10 aa, Cowpea mosaic virus
SHCYISHSFW

SEQ ID NO: 88 - protein, 5 aa, Cowpea mosaic virus
CLWFL

SEQ ID NO: 89 - protein, 4 aa, Cowpea mosaic virus
EFSS

SEQ ID NO: 90 - protein, 5 aa, Cowpea mosaic virus
NCSIC

SEQ ID NO: 91 - protein, 26 aa, Cowpea mosaic virus
GKMYFGFLPTRVCHCLVNTSKPQNHT

SEQ ID NO: 92 - protein, 12 aa, Cowpea mosaic virus
SRWLSLPICNYS

SEQ ID NO: 93 - protein, 9 aa, Cowpea mosaic virus
YNRYNLRRF

SEQ ID NO: 94 - protein, 8 aa, Cowpea mosaic virus
SWGQACWH

SEQ ID NO: 95 - protein, 7 aa, Cowpea mosaic virus
GFLWYRF

SEQ ID NO: 96 - protein, 15 aa, Cowpea mosaic virus
WFPLAWSYSTRTCLC

SEQ ID NO: 97 - protein, 5 aa, Cowpea mosaic virus
SLRCV

SEQ ID NO: 98 - protein, 5 aa, Cowpea mosaic virus
PMYDS

SEQ ID NO: 99 - protein, 14 aa, Cowpea mosaic virus
HSSCSIFRRYSSNF

SEQ ID NO: 100 - protein, 10 aa, Cowpea mosaic virus
LNQRQNNSCW

SEQ ID NO: 101 - protein, 7 aa, Cowpea mosaic virus
QLEYAHL

SEQ ID NO: 102 - protein, 21 aa, Cowpea mosaic virus
SSNYECLAYCCLEIWNYSCST

SEQ ID NO: 103 - protein, 23 aa, Cowpea mosaic virus
GCWCQKSRLGWSSLCLPAPVHEP

SEQ ID NO: 104 - protein, 18 aa, Cowpea mosaic virus
CADICDLTTWFCHVELLF

SEQ ID NO: 105 - protein, 5 aa, Cowpea mosaic virus
YHRAE

SEQ ID NO: 106 - protein, 14 aa, Cowpea mosaic virus
ICRKPMGQSDHLVS

SEQ ID NO: 107 - protein, 12 aa, Cowpea mosaic virus
MCCYQSQTNTAI

SEQ ID NO: 108 - protein, 7 aa, Cowpea mosaic virus
GQHALRS

SEQ ID NO: 109 - protein, 24 aa, Cowpea mosaic virus
FQGCRQYPDAPISTVNGNSTVIKV

SEQ ID NO: 110 - protein, 4 aa, Cowpea mosaic virus
VSGY

SEQ ID NO: 111 - protein, 4 aa, Cowpea mosaic virus
TLQA

SEQ ID NO: 112 - protein, 11 aa, Cowpea mosaic virus
CYGWTHCYCCL

SEQ ID NO: 113 - protein, 587 aa, Cowpea mosaic virus
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQGPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPLLKFRFRDIERSKRSVMVGHTATAA

SEQ ID NO: 114 - protein, 67 aa, Cowpea mosaic virus
WSKTCLPFLWMIQAQFVVLCLTQNSHKLEFCCPRLWLVVMCYWMSISMMWSMDKILELLSLFCAPML

SEQ ID NO: 115 - protein, 13 aa, Cowpea mosaic virus
QLPPTFLTTRVVV

SEQ ID NO: 116 - protein, 16 aa, Cowpea mosaic virus
GVSIVLMFILSALKTP

SEQ ID NO: 117 - protein, 26 aa, Cowpea mosaic virus
RGTQGAKRTSRSHLIQTLVGILGLLR

SEQ ID NO: 118 - protein, 6 aa, Cowpea mosaic virus
VEAELG

SEQ ID NO: 119 - protein, 15 aa, Cowpea mosaic virus
QLFVFRDGPYLLPQM

SEQ ID NO: 120 - protein, 31 aa, Cowpea mosaic virus
TGQLSMRNVSPPFTTWLIVRIGYPLIVGWEN

SEQ ID NO: 121 - protein, 5 aa, Cowpea mosaic virus
LFPRV

SEQ ID NO: 122 - protein, 10 aa, Cowpea mosaic virus
QVRFEGCLFL

SEQ ID NO: 123 - protein, 35 aa, Cowpea mosaic virus
EAVLVRLKLSWPICPIHGYQCGDILEVNFTLKLLK

SEQ ID NO: 124 - protein, 12 aa, Cowpea mosaic virus
ALHILKPLLHFS

SEQ ID NO: 125 - protein, 45 aa, Cowpea mosaic virus
LLVILVMPLVFMRVFLIELFNLLRLRKNVLWFSPNKSLSLLGQHK

SEQ ID NO: 126 - protein, 44 aa, Cowpea mosaic virus
TPEPHLKQMVVPTYMQLFMIVQQVQSPEILILGSSLLALRIFVV

SEQ ID NO: 127 - protein, 14 aa, Cowpea mosaic virus
VLIRVLMVPACLEL

SEQ ID NO: 128 - protein, 16 aa, Cowpea mosaic virus
HKDLFVLKPQMCIAHV

SEQ ID NO: 129 - protein, 13 aa, Cowpea mosaic virus
LALLLLHFQTLQQ

SEQ ID NO: 130 - protein, 4 aa, Cowpea mosaic virus
STAK

SEQ ID NO: 131 - protein, 17 aa, Cowpea mosaic virus
LLLVMTIGIRTFIILQL

SEQ ID NO: 132 - protein, 40 aa, Cowpea mosaic virus
MSCVLLLGNLELFMFNLMLGVLVSKEQIGMVKSLFTCASP

SEQ ID NO: 133 - protein, 10 aa, Cowpea mosaic virus
TLKVMMRGHL

SEQ ID NO: 134 - protein, 8 aa, Cowpea mosaic virus
SHNLVLPC

SEQ ID NO: 135 - protein, 6 aa, Cowpea mosaic virus
TSLLIS

SEQ ID NO: 136 - protein, 50 aa, Cowpea mosaic virus
GRIADLNLPKAHGPIRPPGILNVLLPIPDKYSNLRSTCASILISGLPAIS

SEQ ID NO: 137 - protein, 13 aa, Cowpea mosaic virus
CPHFHCQRKLHRY

SEQ ID NO: 138 - protein, 24 aa, Cowpea mosaic virus
SLGFGILNAPSVVLWLDTLLLLLN

SEQ ID NO: 139 - DNA, 1764 bp, Cowpea mosaic virus
ttaagcagcagtagcagtgtgtccaaccataacactacgcttggagcgttcaatatcccgaaacctaaactttaataacggtggagtttccgttgacagtggaaatgggggcatcaggatattgccggcaaccctgaaattaggatcgaagcgcatgttgacctcaaattgctgtatttgtctgggattggtagcaacacattcaagataccaggtggtctgattggcccatgggctttcggcaaattcaaatccgctattcggccctatgatatcaaaagagaagttcaacatggcagaaccaggttgtgagatcacaaatgtccgcgcatcataactttcagggttcatggactggcgcaggtaaacaaagacttgaccatcccaatctgctcttttgacaccagcacccctaacattaagttgaacatgaatagttccagatttccaagcagcagtacgcaagacattcataattggaggattataaatgtgcgtattccaattgtcatcaccaacaggagttattttgccgttgattaagtcaaaagttactgctgtaacgtctgaaaatggagcaggaggagtgctagctatcatacatgggctatacacatctgaggcttcagcacaaacaggtccttgtgctatagctccaagcaagcgggaaccatcaatacccggattagaacctataccacaaaaatccttaatgccaacaagcttgaccccaagattaaaatctccggagattgtacctgttgtactatcatgaataattgcatataggtagggacaaccatctgcttcaagtgtggttctggggtttacttgtgttgaccaagcagtgacaaactcttgttgggagaaaaccaaagtacatttttcctcaacctcagcaaattgaacaattctatgaggaaaactctcataaaaaccaaaggcatcactaagattaccaaaagctatgagaaatgtaacagtggctttaatatatggagagctcattttagtaacttcaaagtgaagttcacctctaaaatatctccacattgatatccatgaattgggcatattggccaagaaagcttgagtcgcaccagcaccgcctcctatagaaagaggcatccttcgaacctcacttgtcacaccctggggaaaagtcaattttcccatccaacgattaaggggtaaccaattctgacaatcagccaagtggtaaatggtgggctcacatttctcattgacaattgaccagtctagcttggcaatcacatctgtggtaggagataaggtccatcccgaaacacaaataactgtcatcctaactctgcttcgacttatcatctcagcagaccaagaatccccacaagggtttggattaaatgtgaacgagaagttctttttgcaccctgggttccacgtcatggagtcttgagagcagatagtataaacatcagtactatacttacccctcacaccactatttatggccaacatcaaacaacaacccgagttgtcagaaatgttggtggtagctgtcacctttattttgcctgttataacatgggtgcgcaaaaaagcgacagtagctctaaaatcttgtccattgaccacatcatagagatactcatccaataacacatcaccaccagccatagccttggacaacaaaactcgagtttgtgcgaattttgtgtcaagcaaagaaccacgaactgagcttgtatcatccaaagaaagggcaaacaagttttgctccat

SEQ ID NO: 140 - DNA, 5889 bp, Cowpea mosaic virus
tattaaaatcaatacaggttttgataaaagcgaacgtggagaaatccaaacctttctttctttcctcaatctcttcaattgcgaacgaaatccaagctttggttttgctgaaacaaatacacaacgtatactgaatttggcaaatttctctctctctctctgtcattttctttcttctgtcgggactttcttagtcttgacccaacatgggtctcccagaatatgaggccgatagtgaggctttattaagtcaactcactatcgaattcacacccggcatgacagtttcttcattgttggcacaagtcaccactaatgactttcacagtgccattgagttttttgctgcagaaaaagcagtagacattgagggcgttcattacaatgcgtatatgcaacaaattaggaaaaaccctagtttattacgcatttccgtggtagcttatgctttccacgtttcagacatggtagctgagaccatgtcttatgatgtttatgaatttctgtataaacattatgcccttttcatctctaatctggtgaccagaacactcagatttaaagagcttttgctgttctgtaagcagcaatttctggagaaaatgcaagcttcaatagtctgggctccggaacttgagcaatatcttcaagttgaaggggatgctgtggctcaaggagtttcacaactgttatacaagatggtcacttgggtgcccacttttgtcagaggagcagtagactggagcgttgatgcgattttggtcagtttcaggaaacattttgaaaagatggttcaggagtatgtgcccatggctcatcgcgtttgcagttggctgagccaactatgggataagatcgtgcaatggatctcacaagcaagtgagaccatgggttggtttctagatggttgtcgggatttgatgacttggggaattgccactctcgcaacatgtagtgctctctccctggttgagaagctgttagtcgcaatgggttttctggttgagcctttcggcttgagtggaatcttcttgcggacgggagttgttgcggcagcttgttataactatgggactaattctaagggttttgccgagatgatggctttgttgtcattggcggctaactgtgtctctacagttatagttggtggctttttccctggtgaaaaggacaatgcacagagtagtcctgttatcctcttagaaggattggctgggcagatgcaaaacttttgtgagactacacttgtcagtgttgggaaaacatgcactgccgtcaatgctatctcaacatgttgtgggaatctgaaagcactggccggaaggatcttgggcatgctcagagattttatctggaagactttgggctttgagaccagatttctagcagatgcatctttgctttttggcgaggatgttgatggatggctcaaagcaatcagtgatctgcgagatcaatttattgccaaatcatactgttcgcaggatgagatgatgcagattttggtgttgcttgaaaagggaaggcagatgcggaaaagtggtctttctaaaggaggcatttctcctgctatcattaatctgattctcaaagggattaatgatcttgaacaattgaaccgcagctgttcagtgcaaggagtaagaggagttaggaaaatgccatttaccattttcttccaaggaaagtcacgcactggtaagagtttgctgatgagtcaggttacaaaggattttcaggatcactatggattgggtggagaaactgtgtacagtagaaatccttgtgatcaatattggagtggatatcggcggcaaccttttgtgctgatggatgattttgccgccgttgttactgagccgtctgctgaggctcagatgatcaatctgatttctagtgctccatatcctttgaatatggctggacttgaagaaaaaggaatttgttttgattctcaatttgtttttgtttccaccaacttcttggaagtatctcctgaagccaaagttagggacgatgaggctttcaagaacaggagacatgtgattgttcaggtttcaaatgatcctgccaaagcatatgatgctgcaaattttgctagcaaccaaatttacaccattttggcatggaaggatggtcgatacaacaccgtgtgcgttattgaggactatgatgagctggtggcatatttgttgactaggagtcaacagcatgctgaagagcaggagaagaatcttgctaacatgatgaagagtgctacatttgaaagtcatttcaaaagtttagttgaagtccttgagctcggttctatgatatctgctggttttgatatcattcggccagaaaaacttcctagtgaagctaaggagaagagagtcctttacagtattccctacaatggggagtattgtaatgcactcattgatgacaattacaatgttacttgctggtttggtgagtgtgttggtaatcctgagcagctctctaagtacagtgaaaagatgcttttgggtgcttatgaatttcttctgtgttctgagagcttgaatgttgtaattcaggcacatttgaaggaaatggtttgccctcaccattatgacaaggagctcaattttattggcaagataggagagacctactatcacaatcagatggtttcaaatatcggctctatgcagaaatggcatcgtgccattctgtttggaattggggttctcttgggaaaggaaaaagagaagacatggtaccaagttcaggttgccaatgttaaacaagctctttacgacatgtacactaaggagattcgtgattggcccatgccgatcaaagtcacctgtggaattgtcttggcagctattgggggtagtgccttttggaaagtgtttcaacaactagtgggaagcggaaatggtccagtattgatgggtgtggctgctggagcattcagtgctgagcctcaaagtagaaagcccaataggtttgatatgcagcaatacaggtacaacaatgttcctctcaagagaagagtttgggcagacgcacaaatgtctttggatcagagtagtgttgctatcatgtctaagtgtagggctaatctggtttttggaggcactaatttgcaaatagtcatggtaccaggaagacgctttttggcatgcaaacatttcttcacccacataaagaccaaattgcgtgtggaaatagttatggatggaagaaggtactatcatcaatttgatcctgcaaatatttatgatatacctgattctgagttggtcttgtactcccatcctagcttggaagacgtttcccattcttgctgggatctgttctgttgggacccagacaaagaattgccttcagtatttggagcggatttcttgagttgtaaatacaacaagtttgggggtttttatgaggcgcaatatgctgatatcaaagtgcgcacaaagaaagaatgccttaccatacagagtggtaattatgtgaacaaggtgtctcgctatcttgagtatgaagctcctactatccctgaggattgtggatctcttgtgatagcacacattggtgggaagcacaagattgtgggtgttcatgttgctggtattcaaggtaagataggatgtgcttccttattgccaccattggagccaatagcacaagcgcaaggtgctgaggaatactttgattttcttccagctgaagagaatgtatcttctggagtggctatggtagcaggactcaaacaaggagtttacataccattacccacaaaaacagcgctagtggagaccccctccgagtggcatttggacacaccatgtgacaaagttcctagcattttagttcccacggatccccgaattcctgcgcaacatgaaggatatgatcctgctaagagtggggtttccaagtattcccagcctatgtctgctctggaccctgagttacttggcgaggtggctaatgatgttctcgagctatggcatgactgcgctgtagattgggacgattttggtgaagtgtctctggaggaagctttgaatggatgtgaaggagtggaatatatggaaaggattccattagcaacttctgagggctttccgcacattctttctagaaatgggaaagaaaaggggaaaagacggtttgttcagggagatgattgtgttgtctcactaattccaggaactactgtagccaaagcttatgaggagttggaagcaagtgcacacagatttgttcccgctcttgttgggattgaatgtccaaaagatgagaagttgcctatgagaaaggtttttgataagcctaagaccaggtgttttaccattttgccaatggaatataatttggtcgttcgtaggaagtttctgaattttgtgcgctttatcatggccaatcgtcacagactcagttgtcaagtgggtattaatccatattcaatggaatggagtcgcttagcagcaaggatgaaagagaaaggcaatgatgtcttgtgttgtgattatagctcattcgatggcttgctttctaagcaagtgatggatgtcattgctagcatgatcaatgaactttgtggtggagaggatcaactcaaaaatgcaaggcgaaacttgttaatggcgtgttgctctaggttggctatttgcaagaatacagtatggagagttgagtgtggtattccttcagggtttccaatgacagtgattgtgaatagcatttttaatgagattctcattcgctatcattacaagaaactcatgcgcgaacaacaagctcctgaactgatggtacagagttttgataaactcatagggctggtgacttatggtgatgataatctgatttcagtgaatgctgttgtgacaccctattttgatgggaagaaattgaagcaatctttggctcagggtggtgtgactatcactgatggtaaggacaaaacaagtttggaacttccttttcgcagattggaagaatgtgattttctcaagagaacttttgttcagaggagcagtaccatctgggacgctccagaggataaggcaagtttgtggtcgcagcttcattatgttaattgcaacaattgtgagaaagaagttgcttatttgactaatgttgttaatgttcttcgtgaactttatatgcatagtcctcgggaagccacagaatttaggaggaaggtcttaaagaaggtcagttggatcactagtggagatttgcctactttggcacaattgcaagagttctatgagtaccagcggcagcaaggtggggcagacaacaatgacacttgtgacttgttaacaagtgtagacttgctaggtcctcctttgtcttttgagaaagaagcgatgcacggatgcaaagtgtctgaagaaatcgtcaccaagaatttggcatattacgatttcaaaaggaaaggtgaggatgaagtggtatttctgttcaatacgctctatcctcagagttcattgcctgatgggtgtcactctgtgacctggtctcagggtagtggaaggggaggtttgcccacacaaagttggatgagctataatataagcaggaaagattctaatatcaacaagattattagaactgctgtttcttcgaagaaacgagtgatattctgtgctcgtgataatatggttcctgttaacattgtagctttgctctgtgctgttagaaacaagctgatgcccactgctgtatctaatgctacacttgtcaaggtgatggaaaatgccaaagctttcaagtttttaccagaagagttcaatttcgctttttctgatgtttaggtaaataatgcttatgtttttgtttgctcctgtttagcaggtcgttccttcagcaagaacaacaaaaatatgtgtttttatt

SEQ ID NO: 141 - protein, 214 aa, Artificial Sequence
  Synthetic sequence
MGPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPLLKFRFRDIERSKRSVMVGHTATAA

SEQ ID NO: 142 - DNA, 126 bp, Artificial Sequence
  Construct used to express VP60 with a His-tag
aggtcaacatgcgcttcgatcctaatttcagggttgccggcaatatcctgatgcccccatttccactgtcaacggaaactccacctgtacccgggcatcaccatcaccatcactagctcgaggcct

SEQ ID NO: 143 - protein, 37 aa, Artificial Sequence
  Synthetic Construct
VNMRFDPNFRVAGNILMPPFPLSTETPPVPGHHHHHH

SEQ ID NO: 144 - DNA, 126 bp, Artificial Sequence
  Construct used to express VP60 with a His-tag
aggcctcgagctagtgatggtgatggtgatgcccgggtacaggtggagtttccgttgacagtggaaatgggggcatcaggatattgccggcaaccctgaaattaggatcgaagcgcatgttgacct

(19) United States
(12) Patent Application Publication
(10) Pub. No.: US 2012/0174263 A1
(43) Pub. Date: Jul. 5, 2012

(54) PRODUCTION OF VIRAL CAPSIDS

(75) Inventors: Keith Saunders, Norwich (GB); George Peter Lomonossoff, Norwich (GB); Frank Sainsbury, Quebec City (CA)

(73) Assignee: PLANT BIOSCIENCE LIMITED, Norwich, Norfolk (UK)

(21) Appl. No.: 13/378,347

(22) PCT Filed: Jun. 15, 2010
(86) PCT No.: PCT/GB10/01183

§ 371 Date: Dec. 14, 2011

Related U.S. Application Data
(60) Provisional application No. 61/186,970, filed on Jun. 15, 2009.

(51) Int. Cl. A01H 5/00; C07K 14/00; C12N 5/04; C12N 15/63; C12P 21/06; C12N 7/06

(52) U.S. Cl. 800/298; 435/69.1; 435/238; 435/410; 435/320.1; 530/350

(57) ABSTRACT

The invention provides methods of producing “empty” RNA virus capsids (e.g. from Cowpea mosaic virus) by assembly of viral small (S) and large (L) coat proteins in such a way that encapsidation of native viral RNA is avoided. Aspects of the invention employ in planta expression of capsid components from DNA vectors encoding the S and L proteins or S-L polyproteins including them. Such capsids have utility for the encapsidation or presentation of foreign proteins or desired payloads.

### FIELD OF THE INVENTION

The present invention relates generally to methods and materials for generating ‘empty’ viral capsids in host cells which are do not carry the natural RNA viral genome, and hence are non-infective.

### BACKGROUND OF THE INVENTION

Cowpea mosaic virus (CPMV) is a bipartite single-stranded, positive-sense RNA virus and is the type member of the genus comovirus which is classified with genera faba- and nepovirus as genera within the family Comoviridae. CPMV has a genome consisting of two molecules of positive-strand RNA (RNA-1 and RNA-2) which are separately encapsidated in icosahedral particles of approximately 28 nm diameter. These particles contain 60 copies each of a Large (L) and Small (S) protein arranged with pseudo T=3 (P=3) symmetry (Lomonossoff and Johnson, 1991; Lin et al., 1999). The L and S proteins are situated around the 3- and 5-fold symmetry axes and contain two and one β-barrel, respectively. The S protein can exist in two forms, fast and slow, depending on whether the C-terminal 24 amino acids are present (Taylor et al., 1999)

Both CPMV genomic RNAs are expressed through the synthesis and subsequent processing of large precursor polyproteins (for a review, see Goldbach and Wellink, 1996).

RNA-1 encodes the proteins involved in protein processing and RNA replication (Lomonossoff & Shanks, 1983). The polyprotein encoded by RNA-1 self-processes in cis through the action of the 24K proteinase domain to give the 32K proteinase co-factor, the 58K helicase, the VPg, the 24K proteinase and the 87K RNA-dependent RNA-polymerase.

RNA-2 is translated to give a pair of polyproteins, (the 105K and 95K proteins) as a result of initiation at two different AUG codons at positions 161 and 512. These polyproteins are processed by the RNA-1-encoded 24K proteinase in trans at 2 sites to give the 58K/48K pair of proteins (which differ only at their N-terminus) and the mature L and S coat proteins (FIG. 1a).

Two cleavages of the 95/105K polyprotein are required to produce the mature L and S coat protein—at a Gln/Met site between the 58/48K protein and the L coat protein and at a Gln/Gly site between the L and S coat proteins. Cleavage at the 58/48K-L junction requires not only the action of the 24K proteinase but is also dependent on the presence of the RNA-1-encoded 32K proteinase co-factor (Vos et al., 1988). Cleavage at this site leads to the production of an L-S fusion protein (termed VP60) which has been proposed as the immediate precursor of the mature L and S proteins (Franssen et al., 1982; Wellink et al., 1987).

Detailed knowledge of the structure of the CPMV particle, coupled with its robustness, has led to it being extensively used in bio- and nanotechnology (for a recent reviews, see Steinmetz et al., 2009; Destito et al., 2009).

However, though much is known about the structure and properties of the mature CPMV particle, relatively little is known about the mechanism of virus assembly. It has, to date, proved impossible to develop an in vitro assembly assay since the L and S proteins isolated from virions are insoluble in the absence of denaturants (Wu and Bruening, 1971).

To date, CPMV particles have generally been isolated from infected plants. Yields of up to 1 g of virus per kg of starting leaf material are readily obtained from typical CPMV infections. In such natural preparations approximately 90% of the particles contain either the viral RNA-1 or RNA-2. The presence of viral RNA within the particles has several undesirable consequences for their technological application. These include:
The virus preparations retain their ability to infect plants and spread in the environment.
While CPMV RNAs have not be shown to be capable of replication in mammalian cells, uptake of particles does occur both in vitro and in vivo, raising biosafety concerns if RNA-containing particles are used for veterinary or medical applications
The presence of the RNA within the particles precludes the incorporation of additional material within the CPMV capsids.

To address these issues, attempts have been made to inactivate or eliminate the viral RNAs.

Langeveld et al., 2001 reported a canine parvovirus vaccine based on a recombinant chimeric CPMV construct (CPMV-PARVO1). This was inactivated by UV treatment to remove the possibility of replication of the recombinant plant virus in a plant host after manufacture of the vaccine.

Rae et al., 2008 used UV irradiation to crosslink the RNA genome within intact particles. Intermediate doses of 2.0-2.5 J/cm2 were reported to maintain particle structure and chemical reactivity, with cellular binding properties being reported to be similar to CPMV-WT.

Ochoa et al., 2006 reported a method to generate a CPMV empty capsids from their native nucleoprotein counterparts by removing the encapsidated viral genome by chemical means.

Phelps et al., 2007 reported chemical Inactivation and purification of cowpea mosaic virus-like particles displaying peptide antigens from Bacillus anthracis.

However, all these inactivation or purification processes have to be carefully monitored as they risk altering the structural properties of the particles.

Shanks & Lomonossof (2000) describes how regions of RNA-2 of Cowpea mosaic virus (CPMV) that encoded the L and S coat proteins could be expressed either individually or together in Spodoptera frugiperda (sf21) cells using baculovirus vectors. Co-expression of the two coat proteins from separate promoters in the same construct resulted in the formation of virus-like particles whose morphology closely resembled that of native CPMV virions. The authors concluded that the expression of the coat proteins in insect cells could provide a fruitful route for the study of CPMV morphogenesis.

A presentation was given at the ASSOCIATION OF APPLIED BIOLOGISTS (AAB) “Advances in Virology” meeting, University of Greenwich, UK held on 11-12 Sep. 2007, entitled “Cowpea mosaic virus from insect cell culture; a template for bionanotechnology” by K SAUNDERS, M SHANKS & G P LOMONOSSOFF (John Innes, Norwich, UK). This presentation described possible uses of CPMV produced from insect cell culture in bionanotechnology. It was reported that virus like particles could result from co-expression of the L and S coat proteins in insect cells. Additionally, insect cells co-infected with RNA1 and RNA2 derived constructs produced high molecular weight bands when probed with suitable antibodies.

Wellink et al., 2006 reported studies in which the coding regions for CPMV capsid proteins VP37 (L) and VP23 (S) were introduced separately into a transient plant expression vector containing an enhanced CaMV 35S promoter. Significant expression of either capsid protein was reportedly observed only in protoplasts transfected simultaneously with both constructs. Immunosorbent electron microscopy apparently revealed the presence of virus-like particles in extracts of these protoplasts. An extract of protoplasts transfected with both constructs together with RNA-1 was able to initiate a new infection, which was interpreted as showing that the two capsid proteins of CPMV can form functional particles containing RNA-1 and that the 60-kDa capsid precursor is not essential for this process.

Interestingly, when Wellink and co-workers attempted to generate particles from a construct (pMMB110) encoding a hybrid polyprotein comprising a 24 kDa proteinase fused to VP60 (the capsid proteins precursor) no particles were found. Wellink and co-workers were unclear why no virus like particles are formed in pMMB110-transfected protoplasts, and noted that the amount of capsid proteins present in these cells was similar to the amount found in the cotransfected cells. The authors suggested that the conformation of the coat proteins produced in this manner may not have been correct to permit assembly. Alternatively, it may indicate that the processing of the artificial precursor was insufficiently precise, since processing by the 24K proteinase is less specific in cis than in trans (Clark et al., 1999).

This difficulty in mimicking the situation plants which occurs during a virus infection (where the mature L and S proteins are both produced by proteolytic processing of the RNA-2-encoded polyprotein) is consistent with earlier experiments with plants transgenic for VP60, which showed that it could not assemble into VLPs (Nida et al., 1992). Likewise attempts to examine the role of VP60 have been further hampered by the fact that it only accumulates to very low levels during infection of plants (Rezelman et al., 1989) and that cleavage at the L-S site only occurs at very low haemin concentration in reticulocyte lysates (Bu and Shih, 1989).

At a presentation on 1 to 3 Apr. 2009 given in Harrogate, UK (“Advances in Plant Virology” held by the Assoc, of Applied Biologists in conjunction with the Society for General Microbiology) one or more of the present inventors described proteolytic processing of the CPMV coat polyprotein precursor and formation of virus-like particles in insect cell culture.

The authors of the presentation attempted to define the minimum requirements for capsid formation, and produced virus-like particles in which the S protein was of the slower migrating form following the co-expression of VP60 (consisting of a fused L-S protein), with the 24K proteinase. Thus it was concluded that the movement protein expressed at the amino terminus of the coat protein precursor polyprotein (P105/P95) was not essential for capsid formation. In contrast both the faster and slower migrating S protein forms were present in virus-like particles as a consequence of the co-expression of VP60 with the amino terminal portion of RNA 1. This suggested that the 32K processing regulator expressed within the amino terminal region of RNA1, in addition to the 24K proteinase, had a role in the processing of the S coat protein but was also non-essential for virus-like particle formation.

Thus it can be seen that at the priority date, some steps had been taken to form CPMV virus-like particles (VLPs) in both cowpea protoplasts (Wellink et al., 1996) and Spodoptera frugiperda (Sf21) insect cells (Shanks and Lomonossoff, 2000) by the co-expression of the individual L and S coat proteins. However in both cases the yield of assembled particles was low. Additionally, problems were reported in using polyprotein precursors, particularly in plant cells (Wellink et al., 1996).

PCT/GB2009/000060 was filed but not published prior to the presently claimed priority date. It describes the so called CPMV “HT” high-expression system. It is noted that it may be used in the transient format in N. benthamiana to co-express the CPMV S and L coat proteins for assembly into virus-like particles.

Part of the work described herein was published after the presently claimed priority date as “Cowpea Mosaic Virus Unmodified Empty Viruslike Particles Loaded with Metal and Metal Oxide” Aljabali, Sainsbury, Lomonossoff, & Evans: Small V6, I7, pp 818-821.

### SUMMARY OF INVENTION

The present invention concerns the use of host cells to produce ‘empty’ capsids using a high-yield expression system in combination with heterologous nucleic acid encoding the L and S coat proteins. In the description below these ‘empty’ capsids, where devoid or nearly devoid of ‘native’ RNA, may be referred to “eVLPs” for brevity.

To investigate the requirements for VLP formation when the mature L and S proteins are produced by proteolytic processing of a precursor in trans, the present inventors first examined the processing of CPMV RNA-2 polyprotein by the RNA-1-encoded 24K proteinase in insect cells. The results showed that VLPs were efficiently produced when the L and S proteins are released from either the full-length RNA-2 polyproteins or from VP60.

However, while processing and VLP formation from the full-length RNA-2 polyproteins required the simultaneous presence of both the 32K co-factor and the 24K proteinase, the inventors showed that processing from VP60 required just the 24K proteinase and gives rise to very efficient VLP formation.

In separate experiments, agroinfiltration of the VP60 and 24K proteinase constructs into plants also gave rise to VLPs demonstrating that this approach is suitable for the generation of empty particles for use in bio- and nanotechnology. Using the VP60 with the 24 kDa proteinase ensures that the L and S proteins are produced in exactly equal amounts, as they are found in the natural capsid.

The inventors have also shown that encoding VP60 and 24K on a single construct gave rise to VLPs at even higher yields than those obtained using separate constructs.

Additionally, the present inventors have shown that expressing the separate L and S proteins in plants using a high-yield expression system such as the “CPMV-HT” system also results in the formation of empty capsids.

In preferred embodiments of the invention, capsids are prepared from the coat protein precursor VP60 through the action of the CPMV 24 kDa proteinase in planta. Elimination of infectivity by irradiation with ultraviolet light or chemically treatment risks altering the structural properties of the particles. The use of plants inoculated with constructs encoding VP60 and the 24K proteinase to produce non-infectious empty capsids circumvents this problem.

Additionally, producing empty particles in this manner rather than through an infection process has the advantage that the particles no longer need to be competent at packaging RNA or spreading within plant tissue. Accordingly the systems of the present invention extend the range of modifications that it is possible to introduce into the coat proteins, thereby extending the range of their applications.

Thus in one aspect there is provided a method of producing RNA virus capsids in a host cell, which capsids are incapable of infection of the host cell, which method comprises:

(a) introducing one or more recombinant nucleic acid (generally DNA) vectors into the host cell or an ancestor thereof, wherein said one or more vectors comprise:
(i) a first nucleotide sequence encoding a polyprotein which can be proteolytically processed in the host cell to viral S and L coat proteins for assembly in the host cell into viral capsids; and
(ii) a second nucleotide sequence encoding a proteinase capable of said proteolytically processing;
   (b) permitting expression of said polyprotein and proteinase from said first and second nucleotide sequences,
such that the polyprotein is proteolytically processed in the host cell to viral S and L coat proteins which assemble in the host cell into viral capsids;

Preferred vectors for use in the invention are high-level expression vectors, such as the CPMV-HT (“hyper translatable”) vectors described in prior-filed patent application PCT/GB2009/000060 or Sainsbury & Lomonossoff 2008.

As noted above the first and second nucleotide sequences may be on the same or different vectors (cf. compare FIGS. 8 and 10). In some preferred embodiments they are on the same vector and hence only one vector need be introduced into the cell.

Typically the polyprotein includes a cleavage site naturally recognised by a proteinase from the same or a closely related RNA virus. However as described below, in other embodiments the cleavage site mayfrom an unrelated virus or source, and a proteinase which is specific for that site is used.

In another aspect there is provided a method of producing RNA virus capsids in a host cell, which capsids are incapable of infection of the host cell, which method comprises:

(a) introducing one or more recombinant nucleic acid (generally DNA) vectors into the host cell or an ancestor thereof, wherein said one or more vectors comprise:
(i) a first nucleotide sequence encoding a viral S coat protein; and
(ii) a second nucleotide sequence encoding a viral L coat protein, each being present in a high-level expression vector,
   (b) permitting expression of said S coat protein and L coat protein from said first and second nucleotide sequences,
such that S and L coat proteins are assembled in the host cell into viral capsids.

As above the first and second nucleotide sequences may be on the same or different vectors.

Again the preferred high-level expression vector is the CPMV-HT vector. The expression of separate L and S proteins permits the relative amounts to be varied, where that is desired—for Example if they are modified such as to alter the standard 60:60 ratio present in wild-type capsids.

Typically the RNA virus is a bipartite RNA virus will be a comovirus such as CPMV. All genera of the family Comoviridae appear to encode two carboxy-coterminal proteins. The genera of the Comoviridae family include Comovirus, Nepovirus, Fabavirus, Cheravirus and Sadwavirus. Comoviruses include Cowpea mosaic virus (CPMV), Cowpea severe mosaic virus (CPSMV), Squash mosaic virus (SqMV), Red clover mottle virus (RCMV), Bean pod mottle virus (BPMV). The sequences of the RNA-2 genome segments of these comoviruses and several specific strains are available from the NCBI database as described in PCT/GB2009/000060.

The host cell may be present in cell culture or in a host organism such as a plant. In such cases the method may further comprise harvesting a tissue (e.g. leaf) in which the CPMV capsids have been assembled, and optionally isolating them from the tissue.

As described below, the present inventors have further devised an improved protocol for extracting or isolating empty CPMV capsids from leaf tissues which omits the previously used organic solvent extraction step. In conjunction with the other methods herein (for example in which the first and second nucleotide sequences are on the same vector), the protocol can provide yields of up to 0.2 g/Kg leaf tissue (i.e. 0.02% w/w) or more.

In another aspect there is provided a gene expression system for producing CPMV capsids in a host cell, which system comprises one or more recombinant nucleic acid vectors (generally DNA, high-level expression vectors), wherein said one or more vectors comprise:
(i) a first nucleotide sequence encoding a polyprotein which can be proteolytically processed in the host cell to CPMV S and L coat proteins for assembly in the host cell into CPMV capsids; and
(ii) a second nucleotide sequence encoding a proteinase capable of said proteolytically processing.

As above the first and second nucleotide sequences may be on the same or different vectors.

In another aspect there is provided a method comprising the step of introducing the gene expression system into the host cell or organism.

In other aspects there are provided CPMV capsids, particularly those which are essentially free of CPMV RNA, for example as obtainable using methods herein.

In any of the aspects described herein the capsids may include a payload which may be, by way of non-limiting example, a nucleic acid (e.g. silencing agent such as siRNA), protein, carbohydrate, or lipid, a drug molecule e.g. a chemotherapeutic, or an inorganic material such as a heavy metal or salts thereof. The payload may or may not be fluorescent. Internal mineralisation using inorganic materials such as cobalt or iron oxide is demonstrated in the Examples below. As noted elsewhere herein, the capsids may themselves be empty, but modified e.g. to present foreign protein sequences as part of the L or S sequences. The inventors have shown, for example, that the C-terminus of VP60 can be modified to carry foreign sequences without impairing its ability to form eVLPs.

In the practice of the invention, the host cell will be eukaryotic host, which is typically a plant or in insect. Preferred hosts are plants. The vectors or nucleotide sequences described above may thus be employed transiently or incorporated into stable transgenic plants. Such hosts form further aspects of the invention, which thus provides:
A host cell organism obtained or obtainable by a method described above.
A host organism transiently transfected with a gene expression system as described herein.
A transgenic host organism stably transformed with a gene expression system as described herein.

To avoid packaging of naturally infective RNA within the capsids, the nucleic acid vectors of the invention do not encode both the native RNA1 and RNA2 genome of CPMV.

Thus at least one of the native RNA genomes will be absent, or modified such that no infectious virus is produced.

Most preferably, the RNA-2 of the system is truncated such that no infectious virus is produced.

Where an entire native 95/105 protein is encoded by the RNA-2 derived nucleic acid, then preferably the region encoded by the 5′ half of RNA-1 (both the 32 kDa and 24 kDa proteins) would be included, but preferably not the 3′ portion encoding the remaining proteins.

Nevertheless, preferably the first nucleotide sequence encoding the polyprotein will not encode the 32K movement protein which is encoded by the native RNA2 (cf. Greenwich disclosure discussed supra). This movement protein expressed at the amino terminus of the coat protein precursor polyprotein is not essential for capsid formation.

In the invention the proteinase, which is typically a CPMV native 24K proteinase, is generally not expressed as part of the same polyprotein as the L-S polyprotein (cf. Wellink et al. disclosure discussed supra wherein no particles were produced). Rather the L and S proteins are produced by proteolytic processing of a polyprotein precursor in trans.

Preferably the polyprotein comprises only the L and S coat proteins, as exemplified for example by the “VP60” protein described herein. As demonstrated by the inventors, processing of the VP60 protein does not require the CPMV 32K proteinase co-factor. Rather, the CPMV 24K proteinase alone can efficiently process VP60. Furthermore, the L and S proteins resulting from in trans proteolytic processing of the precursor polyprotein, can assemble into CPMV capsids.

It will of course be appreciated that the L and S coat proteins themselves may be genetically modified using conventional techniques to incorporate additional features or activities according to the desired purpose of the capsids—for example epitopes, binding entities and so on. Chemical modification after production is also encompassed by the present invention.

Some particular embodiments of the invention will now be described in more detail.

### Capsids

The invention may be utilised to produce “empty” CPMV capsids, by which is meant that they are essentially free of native CPMV RNA which would be present in capsids using conventional prior art techniques and which would lead to infective particles. Generally they will also be free of unwanted cellular nucleic acids. The term “empty” is therefore used for simplicity since it will be well understood by those skilled in the art. Nevertheless it will be appreciated from the present disclosure that the “empty” capsids of the invention may be used to carry a non-natural payload. This is discussed in more detail below.

As used herein, the terms “capsids” and “virus-like particles” (or “VLPs”) are used interchangeably unless context demands otherwise.

“Essentially CPMV RNA-free” refers to a capsid which contains little or no CPMV-derived RNA, and in particular does not encapsulate CPMV RNA which is capable of infection of a plant. Thus the need for irradiation with ultraviolet light or chemical treatment is obviated.

Preferably the method may be used to produce CPMV capsids of which at least 50, 60, 70, 80, 90, 95, 96, 97, 98, or 99% of the capsids are essentially CPMV RNA-free as judged by sucrose gradient density analysis (see Example 5). Particles which are essentially CPMV RNA-free will generally sediment to a position characteristic of Top' components produced during a natural infection.

It will be understood that in certain embodiments of the invention it may be desirable to use the capsids to actually deliver artificial RNAs (such as siRNAs) carrying the appropriate encapsidation signals. The packaging of such artificial RNAs (which will be encoded by nucleic acid introduced into the cell or ancestor thereof specifically for this purpose, and will not consist of natural RNA1 or RNA2 or endogenous cellular mRNA) forms one aspect of the invention.

By contrast, in natural preparations of CPMV particles, approximately 90% of the particles contain the viral either RNA-1 or RNA-2.

### L-S Polyprotein

As noted above, a preferred polyprotein consists essentially of the L and S proteins (optionally modified). VP60 is an example of such a polyprotein. In the Examples below translation iniation was designed to occur from the methionine which forms the N-terminal residue of the L protein, with termination occurring at the natural stop codon downstream of the S protein.

In embodiments of the invention, the S protein may or may not include the 24 carboxyl-terminal amino acids, which are often lost by proteolysis.

Furthermore, in experiments (not shown) the present inventors have demonstrated the substitution of the carboxy-terminal 24 amino acids of VP60 with a hexahistidine sequence and expression of this modified protein (VP60-His) in plants using the CPMV-HT system. The expressed protein was purified from plant extracts in a one-step process using Ni-affinity chromatography.

In other experiments co-infiltration of VP60-His with the CPMV 24K proteinase led to processing to give L and S-His which assembled into eVLPs. These eVLPs could also be purified by Ni-affinity chromatography. This confirms that, by way of non-limiting example, the C-terminus of VP60 can be modified to carry foreign sequences (in this case a His-tag) thus demonstrating the utility of eVLPs as a protein presentation system. This and other example modifications of the L and\or S proteins are discussed in more detail in the section entitled “Utilities for CPMV capsids” below.

By way of non-limiting example, the L or S protein of CPMV can be engineered to display peptides of protective antigens on the surface loop.

Alternatively, the enclosed space in the interior of the capsids may be modified (e.g. to enhance or inhibit accumulation or packaging of a desired or undesired material) by modification of the L protein in regions which are internally presented.

As yet a further alternative, appropriate modification of the proteins can cause the formation of pores in the capsid, where such are desired.

### Proteinasess

As discussed above, the L-S polyprotein includes a cleavage site recognised by a proteinase. Preferably this is one naturally recognised by a proteinase from the same or a closely related bipartite RNA virus (e.g. CPMV 24K proteinase and VP60).

However in other embodiments the cleavage site may be one that is introduced, but originates from an unrelated virus or source, and a proteinase which is specific for that site is used. For example a cleavage site for an unrelated proteinase (e.g. the well known TEV sequence) may be inserted in the polyprotein between the L and S proteins. Those skilled in the art are aware that many viruses use proteolytic processing to achieve expression of their proteins and the cleavages are highly specific. Examples of suitable sequences and proteinases which may be applied in the present invention can be found in Spall, V. E., Shanks, M. and Lomonossoff, G. P. (1997). Polyprotein processing as a strategy for gene expression in RNA viruses. Seminars in Virology 8, 15-23.

Recovery of CPMV plasmids

As discussed in Example 7, the present inventors have further devised an improved protocol for extracting or isolating empty CPMV capsids from leaf tissues which omits the previously used organic solvent extraction step.

Thus a preferred method for extracting or isolating empty CPMV capsids from suitably transformed or treated plants comprises the following steps:

(1) providing plant material from the plant;
(2) homogenising said material;
(3) adding an insoluble binding agent which binds polysaccharides and phenolics;
(4) removing solid matter;
(5) precipitate the virus particles with a polyol;
(6) recovering the polyol precipitate, optionally by centrifugation;
(7) redissolving the pellet in aqueous buffer;
(8) high-speed centrifuging and discarding pelletable material (e.g. 27000 g for 20 mins)
(9) ultracentrifuging and discarding supernatant (e.g. 118,700 g for 150 mins)
(10) resuspending pellet in aqueous buffer;
(11) optionally medium-speed centrifuging and discarding pelletable material (e.g. 10,000 g for 5 mins).

The method may be characterised by not using an organic solvent extraction step.

### Utilities for CPMV Capsids

The observation that VP60 can be used as a precursor in planta as well as in insect cells, provides the means for the generation of significant quantities of empty CPMV capsids. The availability of such particles is of considerable use in bio- and nano-technology.

Reviews of utility of CPMV capsids in bio- and nanotechnology include those of Steinmetz et al., 2009 and Destito et al., 2009. The capsids of the invention may be used in a manner analogous to those described in the art.

For example chemical and genetic modifications on the surface of viral protein cages such as the CPMV can confer unique properties to the virus particles. The enclosed space in the interior of the virus particles further increases its versatility as a nanomaterial and CPMV is increasingly being used as a nanoparticle platform for multivalent display of molecules via chemical bioconjugation to the capsid surface. A growing variety of applications have employed the CPMV multivalent display technology including nanoblock chemistry, in vivo imaging, and materials science.

Chimeric cowpea mosaic virus (CPMV) particles displaying foreign peptide antigens on the particle surface are suitable for development of peptide-based vaccines.

Example utilities are as follows:

RNA-containing CPMV particles from have previously been used extensively to display peptides on the virus surface for immunological and targeting purposes (Destito et al., 2009; Steinmetz et al., 2009). This has been done by inserting the sequences into exposed loops on either the L or S protein. However, there are restrictions concerning the size and sequence of the inserted which is tolerated before the ability of the virus to multiply and spread within plants is impaired (Porta et al., 2003). The current invention obviates the need for replication and spread and therefore allows for a far wider range of peptides, including polypeptides, to be expressed on the virus surface. This expression would is achieved by inserting sequences encoding the desired peptide into loops on the surface of the L and S proteins using conventional molecular biology techniques, and then forming these into capsids according to the present invention.

Chemical conjugation of proteins or other compounds to the viral surface can be achieved by linking them to reactive functional groups on the virus surface. Naturally occurring groups, such as carboxylates provided by the amino acids aspartic and glutamic acid or amino groups provided by lysine residues, on both the L and S proteins have been used to modify wild-type virus particles isolated from plants (Steinmetz et al., 2009). It has also proved possible to introduce amino acids with different functional groups e.g. cysteine with a sulphydryl group while still preserving viral viability. As well as introducing new groups it is also possible to remove them—an example of this is the selective removal of lysine residues (Chatterji et al., 2004). However, the need to retain infectivity has previously limited the number and nature of the amino acids which can be introduced/eliminated. The elimination of the requirement for infectivity means that far more radical changes can be made to the L and S proteins using site-directed mutagenesis to add, remove or change specific amino acids. This increases the range of uses to which CPMV particles can be put.

To date there are no reports of modifications to the inner surface of CPMV particles. It is believed that this is because of the need to retain the RNA-binding properties of the capsids to ensure they encapsidate the viral genome which is a prerequisite for virus viability. In other words, producing virus particles by the normal infection route in plants precludes modifications to the inner surface virus surface. The use of the systems of the present invention ensures that there is no need to retain RNA-binding properties, or to removed RNA prior to encapsidating a “guest” molecule. Rather, the L and S proteins can be modified such as to provide an environment suitable for encapsidating desired molecules, examples of which can be found in Young et al. (2008).

The liberation from the need to retain viral infectivity means that it is possible to envisage making more radical changes to the viral capsid, for example in terms of morphology and permeability, than has hitherto been possible. For example, it may be desired to increase the size of the channel at the 5-fold axis from its wild-type value of 7.5\acute{Å} (Lin et al., 1999) to allow the ingress of larger molecules. Likewise, it may be desired to make the capsid respond to changes in pH and/or ionic environment so that it undergoes structural rearrangements. This would enable guest molecules to be introduced when the virus is in an “open” conformation and then trapped when conditions are changed. It may also be desired to change the size of the virus particles by making changes to the inter-subunit contacts.

Over the past decade or so there has also been a growing interest in the use of viruses as templates, scaffolds and synthons for exploitation in (bio)nanotechnology in areas as diverse as materials science, engineering, electronics, photonics, magnetic storage, catalysis and biomedicine.¹⁻⁹ Plant virus particles having icosahedral symmetry are able to encapsulate nanoparticles within the size and shape constrained viral capsid. For example, host-guest encapsulation of tungstate, vanadate,^{10,11} titania¹² and Prussian blue nanoparticles¹³ has been previously demonstrated within the particles of Cowpea chlorotic mottle virus. This was facilitated, in part, by the ease with which nucleic acid-free empty particles can be obtained by in vitro assembly. As noted above, until now, CPMV has not been used to encapsulate materials as it has been very difficult to obtain empty particles as these comprise only a small fraction (5-10%) of particles produced during an infection. However, as confirmed in the Examples below, using the systems described herein unmodified empty CPMV virus-like particles can be loaded with metal and metal oxide under environmentally benign conditions.

### Vectors and High-Level Expression Vectors

As note above, preferred vectors for use in the invention are high-level expression vectors.

“Vector” as used herein is defined to include, inter alia, any plasmid, cosmid, phage, viral or Agrobacterium binary vector in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication). The constructs used will be wholly or partially synthetic. In particular they are recombinant in that nucleic acid sequences which are not found together in nature (do not run contiguously) have been ligated or otherwise combined artificially. Unless specified otherwise a vector according to the present invention need not include a promoter or other regulatory sequence, particularly if the vector is to be used to introduce the nucleic acid into cells for recombination into the genome.

In embodiments of the invention, a high-level expression system is used. Such systems exist for bacteria (such as E. coli), yeasts (such as Pischia Pastoris), insect cells (through the use of baculovirus-based vectors) or mammalian expression systems (such as CHO cells) or plants (using either transient expression or stable

In plants, high-level expression can most readily achieved using transient expression. Vectors for this purpose can be based on either replicating DNA- or RNA-containing viruses (Lomonossoff and Montague, 2008). Alternatively, the sequences can be expressed from non-replicating constructs in the presence of a suppressor of gene silencing (Sainsbury and Lomonossoff, 2008; Vezina et al., 2009).

Similar systems may also be used in transgenic plants.

A preferred high-level expression vector for use in plants will generally achieve a yield of at least around 100 mg capsids/kg of harvested fresh weight of tissue (typically leaves). Thus the weight % yield of capsids, including payload where applicable, is preferably at least 0.1/1000×100=0.01% but may in other embodiments be at least or between 0.001 and 0.1%, more preferably at least 0.005 or 0.05%. Such yields can readily be achieved as evidenced by the Examples herein.

A preferred high-level expression vector is the CPMV-HT (“hyper translatable”) vectors described in prior-filed patent application PCT/GB2009/000060. The disclosure of PCT/GB2009/000060 is specifically incorporated herein in support of the embodiments using the CPMV-HT system—for example vectors based on pEAQ-HT expression plasmids.

Thus the vectors for use in the present invention will typically comprise an expression cassette comprising:

(i) a promoter, operably linked to
(ii) an enhancer sequence derived from the RNA-2 genome segment of a bipartite RNA virus, in which a target initiation site in the RNA-2 genome segment has been mutated;
(iii) a first or second nucleotide sequence as described above (encoding L-S polyprotein or proteinase);
(iv) a terminator sequence; and optionally
(v) a 3′ UTR located upstream of said terminator sequence.

“Expression cassette” refers to a situation in which a nucleic acid is under the control of, and operably linked to, an appropriate promoter or other regulatory elements for transcription in a host cell such as a microbial or plant cell.

A “promoter” is a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter.

“Enhancer” sequences (or enhancer elements), as referred to herein, are sequences derived from (or sharing homology with) the RNA-2 genome segment of a bipartite RNA virus, such as a comovirus, in which a target initiation site has been mutated. Such sequences can enhance downstream expression of a heterologous ORF to which they are attached. Without limitation, it is believed that such sequences when present in transcribed RNA, can enhance translation of a heterologous ORF to which they are attached.

A “target initiation site” as referred to herein, is the initiation site (start codon) in a wild-type RNA-2 genome segment of a bipartite virus (e.g. a comovirus) from which the enhancer sequence in question is derived, which serves as the initiation site for the production (translation) of the longer of two carboxy coterminal proteins encoded by the wild-type RNA-2 genome segment.

Typically the RNA virus will be a comovirus as described hereinbefore.

For example the enhancer sequence may comprise nucleotides 1 to 507 of the cowpea mosaic virus RNA-2 genome segment sequence shown in Table A, wherein the AUG at position 161 has been mutated as shown in Table B, located downstream of the promoter. As described in PCT/GB2009/000060, it is believed that mutation of the initiation site at position 161 in the CPMV RNA-2 genome segment is thought to lead to the inactivation of a translation suppressor normally present in the CPMV RNA-2. It is further believed that mutations around the start codon at position 161 may have the same (or similar) effect as mutating the start codon at position 161 itself, for example, disrupting the context around this start codon may mean that the start codon is bv-passed more frequently.

In one embodiment of the invention, the enhancer sequence comprises nucleotides 1 to 512 of the CPMV RNA-2 genome segment (see Table A), wherein the target initiation site at position 161 has been mutated. In another embodiment of the invention, the enhancer sequence comprises an equivalent sequence from another comovirus, wherein the target initiation site equivalent to the start codon at position 161 of CPMV has been mutated. The target initiation site may be mutated by substitution, deletion or insertion. Preferably, the target initiation site is mutated by a point mutation.

In alternative embodiments of the invention, the enhancer sequence comprises nucleotides 10 to 512, 20 to 512, 30 to 512, 40 to 512, 50 to 512, 100 to 512, 150 to 512, 1 to 514, 10 to 514, 20 to 514, 30 to 514, 40 to 514, 50 to 514, 100 to 514, 150 to 514, 1 to 511, 10 to 511, 20 to 511, 30 to 511, 40 to 511, 50 to 511, 100 to 511, 150 to 511, 1 to 509, 10 to 509, 20 to 509, 30 to 509, 40 to 509, 50 to 509, 100 to 509, 150 to 509, 1 to 507, 10 to 507, 20 to 507, 30 to 507, 40 to 507, 50 to 507, 100 to 507, or 150 to 507 of a comoviral RNA-2 genome segment sequence with a mutated target initiation site. In other embodiments of the invention, the enhancer sequence comprises nucleotides 10 to 512, 20 to 512, 30 to 512, 40 to 512, 50 to 512, 100 to 512, 150 to 512, 1 to 514, 10 to 514, 20 to 514, 30 to 514, 40 to 514, 50 to 514, 100 to 514, 150 to 514, 1 to 511, 10 to 511, 20 to 511, 30 to 511, 40 to 511, 50 to 511, 100 to 511, 150 to 511, 1 to 509, 10 to 509, 20 to 509, 30 to 509, 40 to 509, 50 to 509, 100 to 509, 150 to 509, 1 to 507, 10 to 507, 20 to 507, 30 to 507, 40 to 507, 50 to 507, 100 to 507, or 150 to 507 of the CPMV RNA-2 genome segment sequence shown in Table A, wherein the target initiation site at position 161 in the wild-type CPMV RNA-2 genome segment has been mutated.

In further embodiments of the invention, the enhancer sequence comprises nucleotides 1 to 500, 1 to 490, 1 to 480, 1 to 470, 1 to 460, 1 to 450, 1 to 400, 1 to 350, 1 to 300, 1 to 250, 1 to 200, or 1 to 100 of a comoviral RNA-2 genome segment sequence with a mutated target initiation site.

In alternative embodiments of the invention, the enhancer sequence comprises nucleotides 1 to 500, 1 to 490, 1 to 480, 1 to 470, 1 to 460, 1 to 450, 1 to 400, 1 to 350, 1 to 300, 1 to 250, 1 to 200, or 1 to 100 of the CPMV RNA-2 genome segment sequence shown in Table A, wherein the target initiation site at position 161 in the wild-type CPMV RNA-2 genome segment has been mutated.

Enhancer sequences comprising at least 100 or 200, at least 300, at least 350, at least 400, at least 450, at least 460, at least 470, at least 480, at least 490 or at least 500 nucleotides of a comoviral RNA-2 genome segment sequence with a mutated target initiation site are also embodiments of the invention.

In addition, enhancer sequences comprising at least 100 or 200, at least 300, at least 350, at least 400, at least 450, at least 460, at least 470, at least 480, at least 490 or at least 500 nucleotides of the CPMV RNA-2 genome segment sequence shown in Table A, wherein the target initiation site at position 161 in the wild-type CPMV RNA-2 genome segment has been mutated, are also embodiments of the invention.

In a preferred embodiment, the promoter is an inducible promoter.

The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

The termination (terminator) sequence may be a termination sequence derived from the RNA-2 genome segment of a bipartite RNA virus, e.g. a comovirus. In one embodiment the termination sequence may be derived from the same bipartite RNA virus from which the enhancer sequence is derived. The termination sequence may comprise a stop codon. Termination sequence may also be followed by polyadenylation signals.

Gene expression cassettes, gene expression constructs and gene expression systems of the invention may also comprise a 3′ untranslated region (UTR). The UTR may be located upstream of a terminator sequence present in the gene expression cassette, gene expression construct or gene expression system. More specifically the UTR may be located downstream of the first or second nucleotide sequence. The UTR may be derived from a bipartite RNA virus, e.g. from the RNA-2 genome segment of a bipartite RNA virus. The UTR may be the 3′ UTR of the same RNA-2 genome segment from which the enhancer sequence present in the gene expression cassette, gene expression construct or gene expression system is derived. Preferably, the UTR is the 3′ UTR of a comoviral RNA-2 genome segment, e.g. the 3′ UTR of the CPMV RNA-2 genome segment e.g. a 3′ UTR which is optionally derived from the same bipartite RNA virus as the enhancer sequence e.g. nucleotides 3302 to 3481 of the cowpea mosaic virus RNA-2 genome segment sequence shown in Table A, located downstream of the expressed first or second nucleotide sequence.

### Preferred Hyper-Translatable Plant Vectors

Where the host is a plant, the promoter used to drive the gene of interest will preferably be a strong plant promoter. Examples of published promoters include:

(1) CAMV p35S
(2) Cassaya Vein Mosaic Virus promoter, pCAS
(3) Promoter of the small subunit of ribulose biphosphate carboxylase, pRbcS

Other strong promoters include pUbi (for monocots and dicots) pActin and the plastocyanin promoter (Vezina et al., 2009).

Preferably the vectors of the present invention which are for use in plants comprise border sequences which permit the transfer and integration of the expression cassette into the plant genome. Preferably the construct is a plant binary vector. Preferably the binary transformation vector is based on pPZP (Hajdukiewicz, et al. 1994). Other example constructs include pBin19 (see Frisch, D. A., L. W. Harris-Haller, et al. (1995). “Complete Sequence of the binary vector Bin 19.” Plant Molecular Biology 27: 405-409).

As described herein, and in PCT/GB2009/000060, the invention may be practiced by moving an expression cassette with the requisite components into an existing pBin expression cassette, or in other embodiments a direct-cloning pBin expression vector may be utilised.

These examples represent preferred binary plant vectors. Preferably they include the CoIEI origin of replication, although plasmids containing other replication origins that also yield high copy numbers (such as pRi-based plasmids, Lee and Gelvin, 2008) may also be preferred, especially for transient expression systems.

As is well known to those skilled in the art, a “binary vector” system includes (a) border sequences which permit the transfer of a desired nucleotide sequence into a plant cell genome; (b) desired nucleotide sequence itself, which will generally comprise an expression cassette of (i) a plant active promoter, operably linked to (ii) the target sequence and\or enhancer as appropriate. The desired nucleotide sequence is situated between the border sequences and is capable of being inserted into a plant genome under appropriate conditions. The binary vector system will generally require other sequence (derived from A. tumefaciens) to effect the integration. Generally this may be achieved by use of so called “agro-infiltration” which uses Agrobacterium-mediated transient transformation. Briefly, this technique is based on the property of Agrobacterium tumefaciens to transfer a portion of its DNA (“T-DNA”) into a host cell where it may become integrated into nuclear DNA. The T-DNA is defined by left and right border sequences which are around 21-23 nucleotides in length. The infiltration may be achieved e.g. by syringe (in leaves) or vacuum (whole plants). In the present invention the border sequences will generally be included around the desired nucleotide sequence (the T-DNA) with the one or more vectors being introduced into the plant material by agro-infiltration.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

Most preferred vectors are the pEAQ vectors of PCT/GB2009/000060 which permit direct cloning version by use of a polylinker between the 5′ leader and 3′ UTRs of an expression cassette including a translational enhancer of the invention, positioned on a T-DNA which also contains a suppressor of gene silencing and an NPTII cassettes. The polylinker also encodes one or two sets of 6× Histidine residues to allow the fusion of N— or C terminal His-tags to facilitate protein purification. As discussed above, the inventors have modified the C-terminus of VP60 to include a His-tag (see FIG. 9) and shown that eVLPS can still be assembled from it. Nevertheless the His tag enables the rapid purification of the VP60 and\or assembled eVLPs by Ni-affinity chromatography.

The presence of a suppressor of gene silencing in such gene expression systems is preferred but not essential. Suppressors of gene silencing are known in the art and described in WO/2007/135480. They include HcPro from Potato virus Y, He-Pro from TEV, P19 from TBSV, rgsCam, B2 protein from FHV, the small coat protein of CPMV, and coat protein from TCV. A preferred suppressor when producing stable transgenic plants is the P19 suppressor incorporating a R43W mutation.

### In Vitro Aspects

As noted above, the present inventors have shown that, using the CPMV-HT system, but in the absence of the proteinase, unprocessed VP60 can be purified from cells (for example using Ni-affinity chromatography where the VP60 includes a His-tag). This VP60 may be utilised in other aspects of the invention which can be performed in vitro whereby purified VP60 (e.g. VP60-His) is cleaved after purification by the addition of a suitable proteinase (e.g. the CPMV 24K proteinase) and permitted to assemble into eVLPs in a non-cellular environment. This may have particular utility for the in vitro encapsidation of foreign material which might not otherwise readily diffuse into “pre-assembled” eVLPs.

Thus in another aspect there is provided a method of producing RNA virus capsids encapsidating a desired payload in vitro, which method comprises:

(a) introducing a recombinant DNA vector into a host cell or an ancestor thereof, wherein said vector comprises a nucleotide sequence encoding a polyprotein which comprises viral small (S) and large (L) coat proteins from said RNA virus,
(b) permitting expression of said polyprotein from said nucleotide sequence, wherein said polyprotein is not proteolytically processed in the host cell to said viral S and L coat proteins,
(c) purifying said polyprotein from said host cell,
(d) contacting said polyprotein in vitro with (i) a proteinase capable of proteolytically processing the polyprotein to said viral S and L coat proteins and (ii) said payload,
such that the viral S and L coat proteins assemble in vitro into viral capsids encapsidating said payload.

Optionally the polyprotein includes a tag (e.g. His-tag) at the N— or C terminal to facilitate protein purification.

The various preferred embodiments of the other aspects of the invention described herein apply mutatis mutandis to the in vitro aspect unless context demands otherwise. Thus as in other aspects of the invention, the RNA virus is preferably a bipartite RNA virus which is preferably a member of the family Comoviridae (e.g. a Comovirus, e.g. CPMV). The nucleotide sequence preferably encodes CPMV VP60 in which one or both of the CPMV S and L proteins is optionally modified by way of sequence insertion, subtitution or deletion. The proteinase is preferably the CPMV 24K proteinase.

### Other Aspects of the Invention

In a further aspect of the invention, there is disclosed a host cell containing a heterologous construct according to the present invention.

Gene expression vectors of the invention may be transiently or stably incorporated into plant cells.

For small scale production, mechanical agroinfiltration of leaves with constructs of the invention. Scale-up is achieved through, for example, the use of vacuum infiltration.

In other embodiments, an expression vector of the invention may be stably incorporated into the genome of the transgenic plant or plant cell.

In one aspect the invention may further comprise the step of regenerating a plant from a transformed plant cell.

Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148). Suitable vectors may include plant viral-derived vectors (see e.g. EP-A-194809). If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

Nucleic acid can be introduced into plant cells using any suitable technology, such as a disarmed Ti-plasmid vector carried by Agrobacterium exploiting its natural gene transfer ability (EP-A-270355, EP-A-0116718, NAR 12(22) 8711-87215 1984; the floral dip method of Clough and Bent, 1998), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11. Ti-plasmids, particularly binary vectors, are discussed in more detail below.

Agrobacterium transformation is widely used by those skilled in the art to transform dicotyledonous species. However there has also been considerable success in the routine production of stable, fertile transgenic plants in almost all economically relevant monocot plants (see e.g. Hiei et al. (1994) The Plant Journal 6, 271-282)). Microprojectile bombardment, electroporation and direct DNA uptake are preferred where Agrobacterium alone is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233).

The particular choice of a transformation technology will be determined by its efficiency to transform certain plant species as well as the experience and preference of the person practising the invention with a particular methodology of choice.

It will be apparent to the skilled person that the particular choice of a transformation system to introduce nucleic acid into plant cells is not essential to or a limitation of the invention, nor is the choice of technique for plant regeneration. In experiments performed by the inventors, the enhanced expression effect is seen in a variety of integration patterns of the T-DNA.

Thus various aspects of the present invention provide a method of transforming a plant cell involving introduction of a construct of the invention into a plant tissue (e.g. a plant cell) and causing or allowing recombination between the vector and the plant cell genome to introduce a nucleic acid according to the present invention into the genome. This may be done so as to effect transient expression.

Alternatively, following transformation of plant tissue, a plant may be regenerated, e.g. from single cells, callus tissue or leaf discs, as is standard in the art. Almost any plant can be entirely regenerated from cells, tissues and organs of the plant. Available techniques are reviewd in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The generation of fertile transgenic plants has been achieved in the cereals such as rice, maize, wheat, oat, and barley plus many other plant species (reviewed in Shimamoto, K. (1994) Current Opinion in Biotechnology 5, 158-162.; Vasil, et al. (1992) Bio/Technology 10, 667-674; Vain et al., 1995, Biotechnology Advances 13 (4): 653-671; Vasil, 1996, Nature Biotechnology 14 page 702).

Regenerated plants or parts thereof may be used to provide clones, seed, selfed or hybrid progeny and descendants (e.g. F1 and F2 descendants), cuttings (e.g. edible parts), propagules, etc.

The invention further provides a transgenic plant (for example obtained or obtainable by a method described herein) in which an expression vector or cassette has been introduced, and wherein CPMV capsids are accumulated.

The invention also provides a plant propagule from such plants, that is any part which may be used in reproduction or propagation, sexual or asexual, including cuttings, seed and so on. It also provides any part of these plants which includes the plant cells or heterologous vectors, expression systems, or capsids described above.

### Nucleic Acids

“Nucleic acid” or a “nucleic acid molecule” as used herein refers to any DNA or RNA molecule, either single or double stranded and, if single stranded, the molecule of its complementary sequence in either linear or circular form.

Typically the nucleic acid vectors of the present invention are DNA vectors, which encode portions of the RNA genome of a bipartite RNA virus—in particular the capsid coat proteins—which are transcribed and translated into said coat proteins in a host cell, optionally as a cleavable polyprotein, and then assembled into capsids.

In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” Is sometimes used. This term, when applied to DNA, refers to a DNA molecule that is separated from sequences with which it is immediately contiguous in the naturally occurring genome of the organism in which it originated.

For example, an “isolated nucleic acid” may comprise a DNA molecule inserted into a vector, such as a plasmid or virus vector, or integrated into the genomic DNA of a prokaryotic or eukaryotic cell or host organism.

The nucleic acid described herein (e.g. of the gene expression system, or having the first or second nucleotide sequence, or providing the enhancer sequence) may thus consist or consist essentially of DNA encoding a portion, or fragment, of the RNA-1 or RNA-2 genome segment of CPMV. For example, in one embodiment the nucleic acid may not encode at least a portion of the coding region of the RNA-1 or RNA-2 genome segment from which it is derived.

The nucleic acid encoding the polyprotein may consist essentially of the coding sequence for the L and S proteins, and the polyprotein may consist essentially of those proteins.

The phrase “consisting essentially of” when referring to a particular nucleotide or amino acid has the following meaning:

When used in reference to an amino acid sequence, the phrase includes the sequence per se and molecular modifications that would not affect the basic and novel characteristics of the sequence or sequences.

When used in reference to a nucleic acid, the phrase includes the sequence per se and minor changes and\or extensions that would not affect the function of the sequence, or provide further (additional) functionality.

### Variants

It will be appreciated by those skilled in the art that the invention may be utilised not only with the specified sequences set out herein, but also by variants of those sequences sharing the requisite biological activity.

Typically variants of the relevant amino acid or nucleic acid sequences set out herein will share at least about 60%, or 70%, or 80% identity, most preferably at least about 90%, 95%, 96%, 97%, 98% or 99% identity with the recited sequence, as well as retaining the biological activity thereof. The relevant biological activities are as follows:

The “polyprotein” must be proteolytically processable to native or mutated S and L coat proteins for assembly in the host cell into capsids. Fore CPMV, these will typically comprise 60 copies each of a Large (L) and Small (S) protein.

The “proteinase” must be capable of proteolytically processing the polyprotein to native or mutated S and L coat proteins.

The “enhancer” sequences is capable of enhancing downstream expression of the polyprotein and\or proteinase.

By way of non-limiting example, the invention may utilise an expression enhancer sequence with at least 70% identity to nucleotides 1 to 507 of the cowpea mosaic virus RNA-2 genome segment sequence shown in Table 1, wherein the AUG at position 161 has been mutated, located downstream of the promoter;

Naturally, changes to the nucleic acid which make no difference to the encoded polypeptide (i.e. ‘degeneratively equivalent’) are included within the scope of the invention.

Identity may be over the full-length of the relevant sequence shown herein, or may be over a part of it, preferably over a contiguous sequence of about or greater than about 20, 25, 30, 33, 40, 50, 67, 133, 167, 200, 233, 267, 300, 333, 400 or more amino acids or codons.

Thus, where the S or L protein has been engineered to incorporate a heterologous sequence (e.g. foreign epitope), the % identity can be assessed based on the S or L originating parts of the sequence, even if these do not run contiguously.

The percent identity of two amino acid or two nucleic acid sequences can be determined by visual inspection and mathematical calculation, or more preferably, the comparison is done by comparing sequence information using a computer program.

An exemplary, preferred computer program is the Genetics Computer Group (GCG; Madison, Wis.) Wisconsin package version 10.0 program, ‘GAP’ (Devereux et al., 1984, Nucl. Acids Res. 12: 387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

TABLE A
The complete CPMV RNA-2 genome segment
(nucleotides 1 to 3481)

| | |
|---|---|
| 1 | tattaaaatc ttaataggtt ttgataaaag cgaacgtggg gaaacccgaa ccaaaccttc |
| 61 | ttctaaattc tctctcatct ctcttaaagc aaacttctct cttgtctttc ttgcatgagc |
| 121 | gatcttcaac gttgtcagat cgtgcttcgg caccagtaca atgttttctt tcactgaagc |
| 181 | gaaatcaaag atctctttgt ggacacgtag tgcggcgcca ttaaataacg tgtacttgtc |
| 241 | ctattcttgt cggtgtggtc ttgggaaaag aaagcttgct ggaggctgct gttcagcccc |
| 301 | atacattact tgttacgatt ctgctgactt tcggcgggtg caatatctct acttctgctt |
| 361 | gacgaggtat tgttgcctgt acttctttct tcttcttctt gctgattggt tctataagaa |
| 421 | atctagtatt ttctttgaaa cagagttttc ccgtggtttt cgaacttgga gaaagattgt |
| 481 | taagcttctg tatattctgc ccaaatttga aatggaaagc attatgagcc gtggtattcc |
| 541 | ttcaggaatt ttggaggaaa aagctattca gttcaaacgt gccaaagaag ggaataaacc |
| 601 | cttgaaggat gagattccca agcctgagga tatgtatgtg tctcacactt ctaaatggaa |
| 661 | tgtgctcaga aaaatgagcc aaaagactgt ggatctttcc aaagcagctg ctgggatggg |
| 721 | attcatcaat aagcatatgc ttacgggcaa catcttggca caaccaacaa cagtcttgga |
| 781 | tattcccgtc acaaaggata aaacacttgc gatggccagt gattttattc gtaaggagaa |
| 841 | tctcaagact tctgccattc acattggagc aattgagatt attatccaga gctttgcttc |
| 901 | ccctgaaagt gatttgatgg gaggcttttt gcttgtggat tctttacaca ctgatacagc |
| 961 | taatgctatt cgtagcattt ttgttgctcc aatgcgggga ggaagaccag tcagagtggt |
| 1021 | gaccttccca aatacactgg cacctgtatc atgtgatctg aacaatagat tcaagctcat |
| 1081 | ttgctcattg ccaaactgtg atattgtcca gggtagccaa gtagcagaag tgagtgtaaa |
| 1141 | tgttgcagga tgtgctactt ccatagagaa atctcacacc ccttcccaat tgtatacaga |
| 1201 | ggaatttgaa aaggagggtg ctgttgttgt agaatactta ggcagacaga cctattgtgc |
| 1261 | tcagcctagc aatttaccca cagaagaaaa acttcggtcc cttaagtttg actttcatgt |
| 1321 | tgaacaacca agtgtcctga agttatccaa ttcctgcaat gcgcactttg tcaagggaga |
| 1381 | aagtttgaaa tactctattt ctggcaaaga agcagaaaac catgcagttc atgctactgt |
| 1441 | ggtctctcga gaaggggctt ctgcggcacc caagcaatat gatcctattt tgggacgggt |
| 1501 | gctggatcca cgaaatggga atgtggcttt tccacaaatg gagcaaaact tgtttgccct |
| 1561 | ttctttggat gatacaagct cagttcgtgg ttctttgctt gacacaaaat tcgcacaaac |
| 1621 | tcgagttttg ttgtccaagg ctatggctgg tggtgatgtg ttattggatg agtatctcta |
| 1681 | tgatgtggtc aatggacaag attttagagc tactgtcgct tttttgcgca cccatgttat |
| 1741 | aacaggcaaa ataaaggtga cagctaccac caacatttct gacaactcgg gttgttgttt |
| 1801 | gatgttggcc ataaatagtg gtgtgagggg taagtatagt actgatgttt atactatctg |
| 1861 | ctctcaagac tccatgacgt ggaacccagg gtgcaaaaag aacttctcgt tcacatttaa |
| 1921 | tccaaaccct tgtggggatt cttggtctgc tgagatgata agtcgaagca gagttaggat |
| 1981 | gacagttatt tgtgtttcgg gatggacctt atctcctacc acagatgtga ttgccaagct |
| 2041 | agactggtca attgtcaatg agaaatgtga gcccaccatt taccacttgg ctgattgtca |
| 2101 | gaattggtta ccccttaatc gttggatggg aaaattgact tttccccagg gtgtgacaag |
| 2161 | tgaggttcga aggatgcctc tttctatagg aggcggtgct ggtgcgactc aagctttctt |
| 2221 | ggccaatatg cccaattcat ggatatcaat gtggagatat tttagaggtg aacttcactt |
| 2281 | tgaagttact aaaatgagct ctccatatat taaagccact gttacatttc tcatagcttt |
| 2341 | tggtaatctt agtgatgcct ttggttttta tgagagtttt cctcatagaa ttgttcaatt |
| 2401 | tgctgaggtt gaggaaaaat gtactttggt tttctcccaa caagagtttg tcactgcttg |
| 2461 | gtcaacacaa gtaaacccca gaaccacact tgaagcagat ggttgtccct acctatatgc |
| 2521 | aattattcat gatagtacaa caggtacaat ctccggagat tttaatcttg gggtcaagct |
| 2581 | tgttggcatt aaggattttt gtggtatagg ttctaatccg ggtattgatg gttcccgctt |
| 2641 | gcttggagct atagcacaag gacctgtttg tgctgaagcc tcagatgtgt atagcccatg |
| 2701 | tatgatagct agcactcctc ctgctccatt ttcagacgtt acagcagtaa cttttgactt |
| 2761 | aatcaacggc aaaataactc ctgttggtga tgacaattgg aatacgcaca tttataatcc |
| 2821 | tccaattatg aatgtcttgc gtactgctgc ttggaaatct ggaactattc atgttcaact |
| 2881 | taatgttagg ggtgctggtg tcaaaagagc agattgggat ggtcaagtct ttgtttacct |
| 2941 | gcgccagtcc atgaaccctg aaagttatga tgcgcggaca tttgtgatct cacaacctgg |
| 3001 | ttctgccatg ttgaacttct cttttgatat catagggccg aatagcggat ttgaatttgc |
| 3061 | cgaaagccca tgggccaatc agaccacctg gtatcttgaa tgtgttgcta ccaatcccag |
| 3121 | acaaatacag caatttgagg tcaacatgcg cttcgatcct aatttcaggg ttgccggcaa |
| 3181 | tatcctgatg cccccatttc cactgtcaac ggaaactcca ccgttattaa agtttaggtt |
| 3241 | tcgggatatt gaacgctcca agcgtagtgt tatggttgga cacactgcta ctgctgctta |
| 3301 | actctggttt cattaaattt tctttagttt gaatttactg ttatttggtg tgcatttcta |
| 3361 | tgtttggtga gcggttttct gtgctcagag tgtgtttatt ttatgtaatt taatttcttt |
| 3421 | gtgagctcct gtttagcagg tcgtcccttc agcaaggaca caaaaagatt ttaattttat |
| 3481 | t |

The start codons at positions 115, 161, 512 and 524 of the CPMV RNA-2 genome segment are shown in bold and underlined.

TABLE B
Oliqonucleotides which can be used in the mutagenesis
of the CPMV RNA-2 sequence

| Oligonu- | | |
|---|---|---|
| cleotide | Sequence | Mutation |
| A115G-F | CTTGTCTTTCTTGCGTGAGCGATCTT | Removes AUG (→GUG) |
| | CAACG | at 115 eliminating |
| A115G-R | CGTTGAAGATCGCTCACGCAAGAAAG | translation from uORF |
| | ACAAG | |
| U162C-F | GGCACCAGTACAACGTTTTCTTTCAC | Removes AUG (→ACG) |
| | TGAAGCG | at 161 eliminating |
| U162C-R | CGCTTCAGTGAAAGAAAACGTTGTAC | translation from AUG 161 |
| | TGGTGCC | while maintaining amino |
| | | acid sequence of uORF |

The mutant nucleotide of the oligonucleotides used in the mutagenesis are shown in bold

### BRIEF DESCRIPTION OF THE DRAWINGS

FIG. 1.

Diagrammatic representation of baculovirus-expressed CPMV protein constructs. Genome organization of CPMV RNA-1 and RNA-2 and the location of the open reading frames cloned into pMFBD. (a) RNA-1 derived constructs driven by the polyhedron promoter, bv-1A and bv-24K. (b) RNA-2 derived constructs cloned behind the p10 promoter, bv-2 including both the 5′ and 3′ untranslated CPMV sequences and bv-VP60. (c) bv-VP60/24K, construct possessing both the 24 K and VP60 genes. VPg, viral protein genome linked.

FIG. 2.

Polyacylamide gel and western blot analysis of extracts of Sf 21 cells infected with 1-3 bv-2; 4, bv-2 and bv-1A; 5, bv-2 and bv-24K; 6, bv-VP60; 7, bv-VP60 and bv-1A; 8, bv-VP60 and bv-24K; 9, bv-2 and bv-1A. H, extracts from healthy cells. (a) detection of CPMV coat protein. (b) membrane probed with antibody prepared against the 58/48K proteins. L and S, large and small coat proteins.

FIG. 3.

Gradient analysis of virus-like particles (VLPs) prepared from CPMV-infected plants and baculovirus-infected Sf21 cells. (a) CPMV; (b) bv-2 and bv-1A; (c), bv-VP60 and bv-1A; (d) bv-VP60/24K; (e) bv-VP60. (f) Gradient peak fractions resolved on a single polyacrylamide gel. 1, bv-2 and bv-1A; 2, bv-VP60 and bv-1A; 3, bv-VP60/24K; 4, bv-VP60. C, CPMV from infected plants. T, top and B, bottom of each gradient.

FIG. 4.

Transmission electron microscopy of particles of wild-type CPMV (a). and samples from the peak gradient fractions of Sf21 cells infected with bv-2 and bv-1A (b), bv-VP60 and bv-1A (c), bv-VP60/24K (d) and bv-VP60 (e). Bars indicate 20 nm.

FIG. 5.

Production of VLPs in N. benthamiana leaves. Top panel: VP60 and 24K proteinase constructs used in plants to produce VLPs. Middle panel: Coomassie Blue-stained SDS-polyacrylamide gel of extracts from plants infiltrated with the indicated constructs. Lane 4 contains a preparation of purified CPMV.

FIG. 6.

Analysis of VLPs purified from plants or insect cells. Upper panel: Coomassie Blue-stained SDS-polyacrylamide gel of purified VLPs. Lower Panel: Agarose gel stained with Coomassie Blue (top) or ethidium bromide (bottom). The samples loaded on the gels are indicated.

FIG. 7.

Western blot showing the processing of VP60 in plants by the 24 kDa proteinase. The blot was probed with an anti-CPMV serum which predominantly recognises the S protein, thus the L protein appears more faint. The lanes are as follows:

### LEFT-HAND PANEL

empty vector (pEAQ-HT): Extract from leaves infiltrated with the empty pEAQ vector; no CPMV-specific bands.

CPMV/L+CPMV/S: Extract from leaves co-infiltrated with pEAQ vectors expressing the separate L and S proteins; capsids are formed but only the S is detected by the antibody.

VP60: Extract from leaves infiltrated with pEAQ vector expressing VP60; no processing occurs due to absence of proteinase, and a protein the size of VP60 accumulates.

VP60+RNA-1: Extract from leaves co-infiltrated with pEAQ vector expressing VP60 and plasmid pBinP-S1NT expressing RNA-1 as a source of the 24 kDa proteinase; processing to give mature L (faint) and S proteins occurs.

VP60+24K: Extract from leaves co-infiltrated with pEAQ vectors expressing VP60 and the 24 kDa proteinase; processing to give mature L (faint) and S proteins occurs.

Middle Panel—S Coat Protein Modified to Contain 19 Amino Acid Insert in βB-βC Loop VP60(FMDV5): Extract from leaves infiltrated with pEAQ vector expressing VP60 into which FMDV sequence has been inserted; no processing occurs due to absence of proteinase and a protein the size of VP60+the insert accumulates.

VP60(FMDV5)+RNA-1: Extract from leaves co-infiltrated with pEAQ vector expressing VP60 with FMDV insert and plasmid pBinP-S1 NT expressing RNA-1 as a source of the 24 kDa proteinase; processing to give mature L (faint) and a modified S protein carrying the FMDV insert occurs.

VP60(FMDV5)+24K: VP60+24K: Extract from leaves co-infiltrated with pEAQ vectors expressing VP60 with the FMDV insert and the 24 kDa proteinase; Processing to give mature L (faint) and S protein with insert occurs.

### Right-Hand Panel

CPMV: Proteins from purified CPMV preparation.

FIG. 8.

The structures of the high-level expression plasmids used for plant expression are shown: pEAQ-HT-CPMV-24K (a) and pEAQ-HT-CPMV-60K (b). The complete sequence is provided as SEQ ID NO.s 1 and 2 respectively.

FIG. 9.

Construct used by the inventors to express VP60 with a His-tag.

FIG. 10.

The structure of a combined high-level expression plasmid used for plant expression is shown as pEAQexpress-VP60-24K. The complete sequence is provided as SEQ ID NO 3.

FIG. 11.

Analysis of eVLPs produced using combined plasmid of FIG. 10 and modified extraction protocol. The TEM image shows eVLPs negatively stained with 2% Uranyl acetate.

FIG. 12.

SDS-PAGE analysis demonstrating that omitting an organic extraction step increases eVLP recovery.

wt: Highly purified wild-type CPMV particles run as a standard;

Lane 1: eVLPs extracted from leaf tissue using an organic clarification step;

Lane 2: eVLPs extracted from the same amount of leaf tissue without the organic clarification step;

Lane 3: Crude extract

FIG. 13

SDS-PAGE analysis demonstrating that the Presence of VP60 and 24K genes in the same T-DNA region enhances eVLP yield. The L and S proteins from particles have been separated by SDS-PAGE using 12% NuPAGE gels stained with Instant Blue Coomassie stain. The intensity of bands on the gel shows that the expression is enhanced at least three-fold if one vector encodes both genes.

### EXAMPLES

### Methods

Plasmid constructions. All CPMV-derived constructs are based on the nucleotide sequences which appear as GenBank Accession nos. NC_{—}003549 (RNA-1) and NC_{—}003550 (RNA-2). The recombinant donor plasmid pFastBac Dual was modified by site-directed mutagensis and oligonucleotide insertion to yield pMFBD. The original HindIII and EcoRI restriction sites were deleted and EcoRI and MluI restriction sites were introduced between the NcoI and XhoI restriction sites. Finally AgeI and HindIII restriction sites were introduced between the poI 10 and polyhedron promoters. The polymerase chain reaction was used to clone a full-length copy, including both the 5′ and 3′ non-coding nucleotide sequences, of CPMV DNA from pBinPS2NT (Liu and Lomonossoff, 2002) into pMFBD via its BbsI and EcoRI restriction sites to yield pMFDB-2. Similarly by PCR, the region of the RNA-2 open reading frame VP60 of pBinPS2NT was cloned into pMFBD via the BbsI and EcoRI restriction sites to yield pMFBD-VP60. The 5′ half of CPMV RNA-1 corresponding to nucleotides 180 to 3857 was obtained by PCR with plasmid pBinPS1 NT as template DNA and cloned into pMFBD via its BamHI restriction site to yield pMFBD-1A. PCR was used to obtain the region of the RNA-1 open reading frame encoding the 24K proteinase sequence from pBinPS1 NT (Liu and Lomonossoff, 2002) and the sequence was cloned into pMFBD and pMFBD-VP60 via the BamHI and SpeI restriction sites to yield pMFBD-24K and pMFBD-VP60/24K, respectively. After sequence verification, all resulting plasmids were transposed into E. coli DH10Bac and the resulting bacmid DNA was introduced into Spodoptera frugiperda (Sf21) cells as recommended by the manufacturers of the Bac-to-Bac Baculovirus Expression Systems (Invitrogen Ltd).

Extraction of total proteins from infected insect cells. Infected Sf21 cells were harvested 2 to 3 days postinfection, by low speed centrifugation, washed in 10 mM sodium phosphate pH 7 recentrifuged and the resulting pellet suspended in 62.5 mM Tris-HCl, pH6.8, 2% SDS.

Purification of VLPs from insect cells. At 3 or 4 days postinfection, infected Sf21 cells were collected by low speed centrifugation and suspended into 100 mM sodium phosphate pH 7, 0.5% NP40 and stirred on ice for 60 minutes. Cell debris was removed by centrifugation at 17,211 g for 15 minutes and the resulting supernatant was centrifuged at 118,706 g for 150 minutes. The virus pellet was suspended in 10 mM sodium phosphate pH 7 and layered onto 5 mL 10-40% sucrose gradient as described by (Shanks & Lomonossoff 2000). The gradients were centrifuged at 136,873 g for 2 hours at 4° C. and 300 μL fractions were collected.

Expression of VLPs in plants. For expression of proteins in plants using the CPMV-HT system (Sainsbury and Lomonossoff, 2008), the sequences encoding VP60 and 24K were amplified from pBinP-NS1 (Liu et al., 2005) and pBinP-S1-NT (Liu and Lomonossoff, 2002), respectively, using oligonucleotides encoding suitable 5′ and 3′ restriction sites (see Example 6).

Endonuclease treated PCR products were inserted into appropriately digested pEAQ-HT resulting in the expression plasmids pEAQ-HT-VP60 and pEAQ-HT-24K (see FIG. 8 and SEQ ID No.s 1 and 2).

Following electroporation of these plasmids into the Agrobacteria tumefaciens strain LBA4404, transient expression in Nicotiana benthamiana was carried out as previously described (Sainsbury and Lomonossoff, 2008).

RNA-1 expression was provided by pBinP-S1-NT.

For small scale soluble protein extraction, infiltrated leaf tissue was homogenized in 3 volumes of protein extraction buffer (50 mM Tris-HCl, pH 7.25, 150 mM NaCl, 2 mM EDTA, 0.1% [v/v], Triton X-100). Lysates were clarified by centrifugation and protein concentrations determined by the Bradford assay. Approximately 20 μg of protein extracts were separated on 12% NuPage gels (Invitrogen) under reducing conditions and electro-blotted onto nitrocellulose membranes. Blots were probed with G49 and an anti-rabbit horseradish peroxidase-conjugated secondary antibody was used (Amersham Biosciences). Signals were generated by chemiluminescence and captured on Hyperfilm (Amersham Biosciences).

Extraction of VLPs from plants. In one method, CPMV VLP purifications were performed on 10-20 g of infiltrated leaf tissue by established methods (van Kammen, 1971). The amount of empty VLPs was estimated spectrophotometrically at a wavelength of 280 nm, by using the molar extinction coefficient for CPMV empty particles of 1.28.

Subsequently, an improved protocol was developed which is described in Example 7.

Electrophoretic analysis of protein. Extracts of infected cells and gradient fractions were analysed by polyacrylamide gel electrophoresis with the NuPAGE system (Invitrogen Ltd). Gels were either stained with Instant Blue (Expedeon Ltd) or transferred to nitrocellulose and probed with anti-CPMV antibodies or an antibody made to a peptide sequence corresponding to the carboxyl-terminal 14 amino acids of the 48K/58K proteins (Holness et al., 1989). Proteins were visualized by detection with conjugated secondary antibody to horse radish peroxidise.

Transmission electron microscopy. Selected gradient fractions were washed in Microcon Ultracel YM 100-kD Spin (Millipore) tubes with water as recommended by the manufacturer. Samples were placed onto pyroxylin and carbon-coated copper grids and negatively stained with 2% uranyl acetate. Grids were examined at 200 kV in an FEI Tecnai20 transmission electron microscope (FEI UK Ltd, Cambridge) and images were obtained using a bottom-mounted AMT XR60CCD camera (Deben UK Ltd, Bury St. Edmunds) at a direct magnification of 80000×.

### Example 1

### Processing of the RNA-2-Encoded Polyproteins in Trans in Insect Cells to Give the L and S Coat Proteins Requires Both the 24K Proteinase and the 32K Proteinase Co-Factor

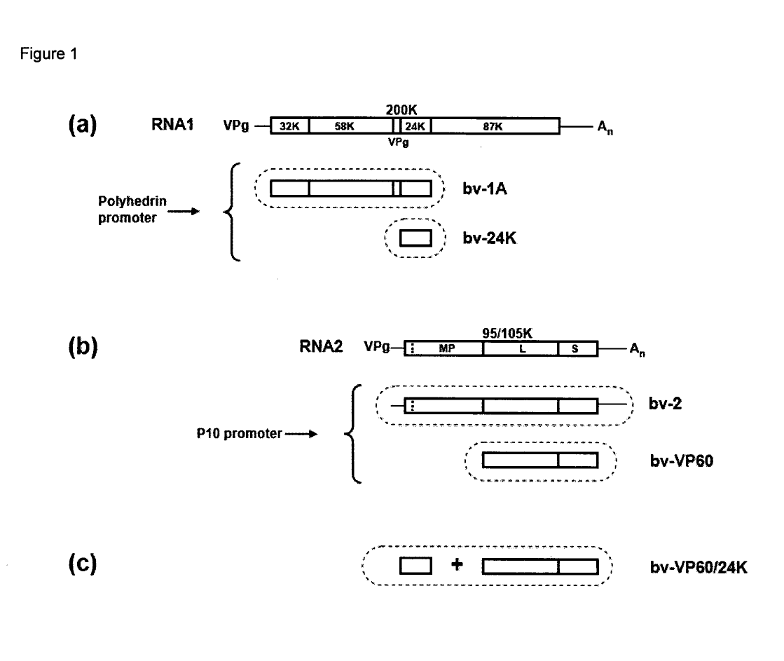

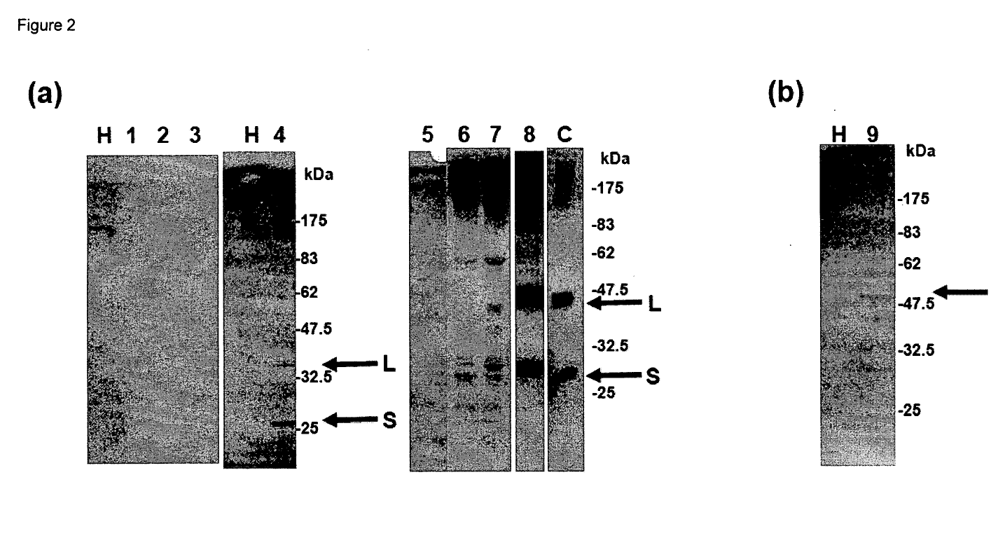

A full-length cDNA clone of RNA 2 was assembled in the baculovirus expression vector pMFBD so that upon transcription the entire nucleotide sequence of RNA-2 would be generated (FIG. 1). Recombinant baculovirus, bv-2, was then produced by transposition of E. coli DH10bac with the pMFBD recombinant plasmid. The resulting recombinant baculovirus DNA was transfected into the Bac-to-Bac expression system (Invitrogen) to test for the expression of both the 105 and 95K CPMV polyprotein precursors. Examination by western blotting of three independently derived samples of Sf21 cells transfected with this construct using an antibody raised against CPMV capsids failed to detect protein products of these sizes (FIG. 2a lanes 1 to 3). This result was not surprising as both the 105 and 95K polyproteins are known to be unstable (Wellink et al., 1989). To achieve processing, a cDNA clone corresponding to nucleotides 207 to 3857 of RNA 1 was constructed in pMFBD (FIG. 1). This construct, bv-1A, encodes the N-terminal portion of the RNA-1-encoded polyprotein and should give rise to the 32K, 58K, VPg and the 24K protein products as a result of the action of the encoded 24K proteinase. Thus it encodes all the factors necessary for the processing of the RNA-2-encoded polyprotein.

Western blot analysis using an antibody raised against CPMV capsids of extracts of Sf21 cells coinfected with bv-2 and bv-1A (FIG. 2a, lane 4) showed the presence of both the L and S coat proteins. This result shows that the 24K proteinase product derived from bv-1A can proteolytic cleave the RNA-2 polyprotein in trans, thereby duplicating the activity of the proteinase found in CPMV infected plants. To confirm processing of the RNA-2 polyprotein had occurred correctly, an extract of Sf21 cells coinfected with bv-2 and bv-1A was probed with an antibody specific to C-terminus of the 58/48K proteins detected the 48K protein product in cells co-infected with bv-2 and bv-1A FIG. 2b lane 9. This confirms that the 24K and 32K protein products can reproduce their in trans activity when expressed in insect cells.

To ascertain whether the 24K proteinase can process the 95 and 105K polyproteins in the absence of the 32K processing regulator, the region of RNA-1 encoding the 24K proteinase was cloned downstream of the polyhedrin promoter to give construct bv-24K. Translation of this construct initiates from the first methionine of the 24K sequence (amino acid 948 of the RNA-1 polyprotein; Wellink et al., 1986) and terminates immediately after the C-terminal glutamine (amino acid 1155). When bv-24K was co-inoculated into Sf21 cells in the presence of bv-2, no products corresponding to the mature L or S protein could be detected on a western blot (FIG. 2a, lane 5). This suggests that in the absence of the 32K processing regulator, the 24K proteinase is ineffective at cleaving the RNA-2 encoded polyproteins.

### Example 2

### Processing of VP60 in Trans to Give the L and S Coat Proteins Requires Only the 24K Proteinase in Insect Cells

To examine whether VP60 can act as a precursor for the mature L and S protein, a cDNA clone, bv-VP60, was constructed which contains the sequence from RNA-2 encoding VP60 (FIG. 1). Translation iniation was designed to occur from the methionine which forms the N-terminal residue of the L protein, with termination occurring at the natural stop codon downstream of the S protein. Western blot analysis using anti-CPMV capsid antiserum of extracts of Sf21 cells transfected with bv-VP60 showed the presence of a protein of approximately 60 kDa which corresponds in size to VP60; a protein of a size which could represent a C-terminally truncated form of the S coat protein was also seen in low abundance (FIG. 2a, lane 6). Co-infection of Sf21 cells with bv-VP60 and bv-1A resulted in the appearance of both the L and S coat proteins as well as some residual VP60 (FIG. 2a, lane 7). To determine whether 24K proteinease can process VP60 by itself, Sf21 cells were co-infected with bv-VP60 and bv-24K and cell extracts were examined by western blotting using anti-CPMV capsid serum. Significant amounts of the mature L and S coat protein were found, indicating that the 24K proteinase alone can efficiently process VP60. Higher levels of the L and S protein were obtained when the VP60 and the 24K sequences were expressed from the same plasmid (construct bv-VP60/24K; data not shown).

### Example 3

### The L and S Proteins Produced by Proteolytic Processing in Trans can Assemble into VLPs in Insect Cells

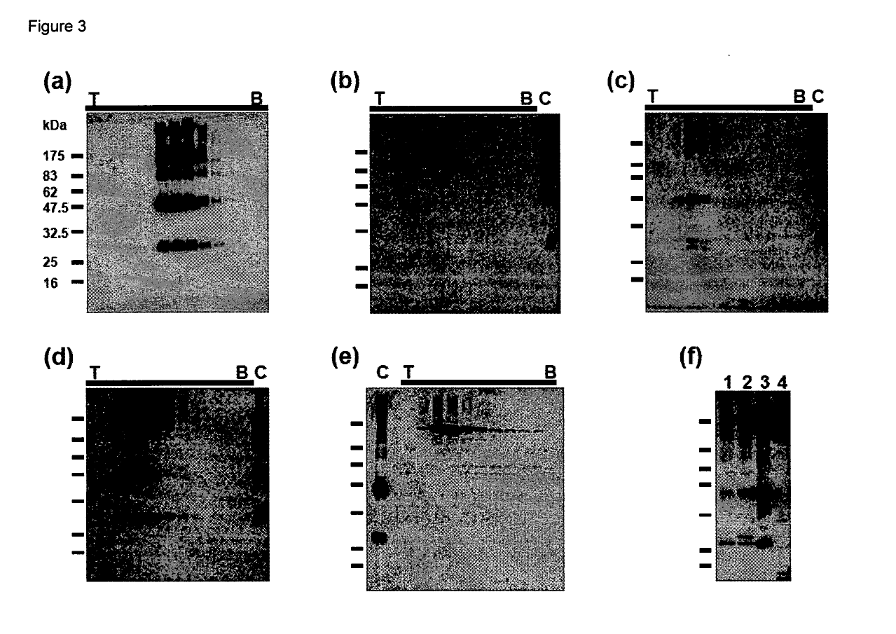

To ascertain whether the L and S proteins resulting from in trans proteolytic processing of precursor polypeptides can assemble into VLPs, extracts of infected cells were prepared and analysed by sucrose gradient density centrifugation. As a control, a preparation of CPMV particles isolated from plants was analysed in parallel. The positions of the L and S proteins in the gradients were determined by western blot analysis, using anti-CPMV, antibodies of samples of each fraction. In the case of CPMV particles isolated from infected plants, most of the L and S protein is found in fractions from the middle of the gradient (FIG. 3a). This represents the sedimentation of the Middle and Bottom components of CPMV, containing RNA-2 and RNA-1, respectively. The small amounts of the L and S proteins in the fractions at the top of the gradient are derived from the relatively low levels of empty particles (Top component) present in a natural preparation of CPMV.

Analysis of extracts prepared from cells infected with bv-2 and bv-1A, with bv-VP60 and bv-1A or with bvVP60/24K showed that in each case the L and S co-sediment suggesting that they have assembled into VLPs (FIG. 3b-d). Moreover, they sediment to a position similar to that of the CPMV empty particles, suggesting that the VLPs produced in insect cells do not encapsidate RNA. Density gradient centrifugation of extracts of cells infected with bv-VP60, which produces uncleaved VP60, showed the presence of a protein of approximately 175 kDa, which was distributed throughout the gradient (FIG. 3e). On the basis of its size, this product could represent an SDS-stable trimer of VP60 which then forms aggregates of a variety of sizes. The peak fractions containing the L and S proteins generated using the various methods of proteolysis were co-run on a single gel (FIG. 3f). While the position of the L protein was consistent in all the samples, the pattern corresponding to the S protein varied. Only the fast migrating form of the S protein is found in cells infected with bv-2 and bv-1A and bv-VP60/24K in comparison to cells infected with bv-VP60 and bv-1A where both the fast and slow migrating forms of the S protein are generated (FIG. 3f).

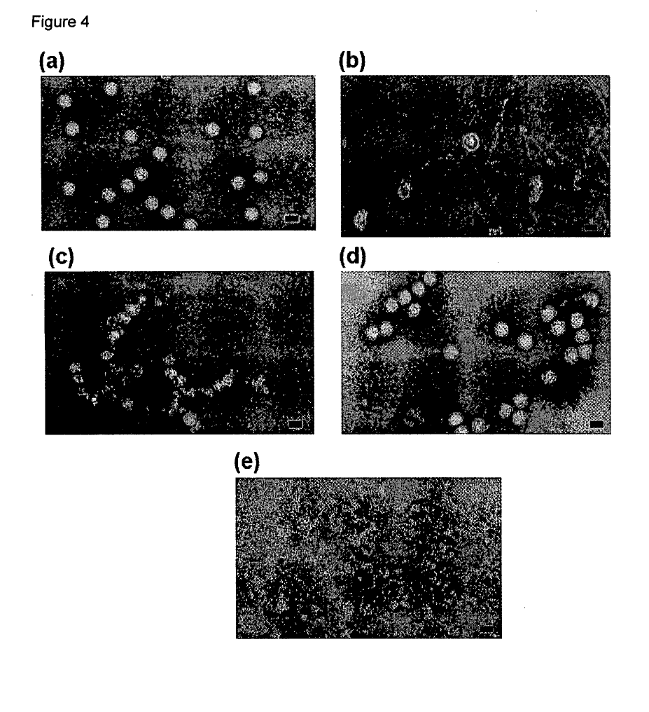

Transmission electron microscopy of the material obtained from the peak fractions containing the L and S proteins of the sucrose gradients of insect cell extracts revealed the presence of virus-like particles (FIG. 4b-d) which were similar in appearance to particles isolated from plants (FIG. 4a). Particles were relatively abundant in extracts from cells infected with bv-VP60/24K compared to extracts from cells co-infected with either bv-2 or bv-VP60 and bv-1A and their appeared to be less background material (FIG. 4, compare panels b and c with panel d). No particles were seen in preparations from extracts of insect cells infected with bv-VP60 alone (FIG. 4e).

### Example 4

### Processing of VP60 by the 24K Proteinase in Plants Leads to VLP Formation

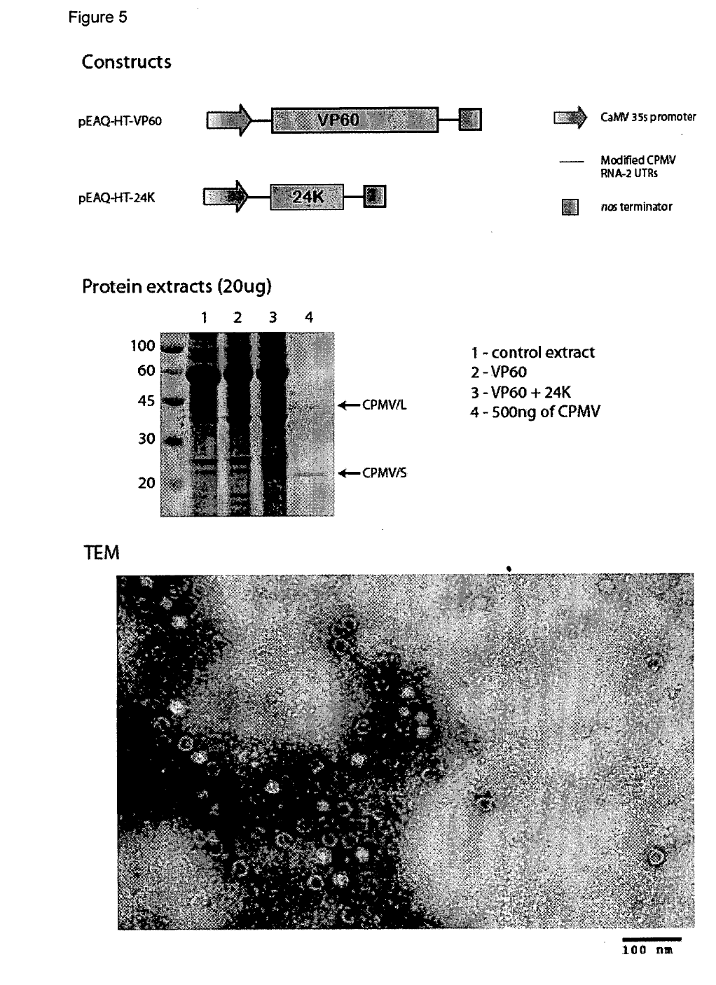

To determine whether the 24K-directed processing of VP60 in insect cells also occurs in plants, we employed a recently developed high-level transient expression system (Sainsbury and Lomonossoff, 2008). This system has been shown to allow the co-expression of multiple proteins from separate plasmids in plant cells using agro-infiltration. To examine the ability of VP60 to act as a precursor to capsid formation in plants, the construct pEAQ-HT-VP60 (FIG. 5) was infiltrated into N. benthamiana leaves in the presence of a construct (pEAQ-HT-24K; FIG. 5) expressing the 24K proteinase. Analysis of protein extracts from infiltrated tissue on SDS/polyacrylamide gels revealed that VP60 is cleaved into the L and S coat proteins in the presence of the 24K proteinase (FIG. 5, middle panel). Potential VLPs resulting from the co-infiltration of leaves with pEAQ-HT-VP60 and pEAQ-HT-24K were purified using the standard CPMV purification protocol (van Kammen, 1971). Electron microscopy revealed the presence of CPMV particles in the resulting material FIG. 5, bottom panel).

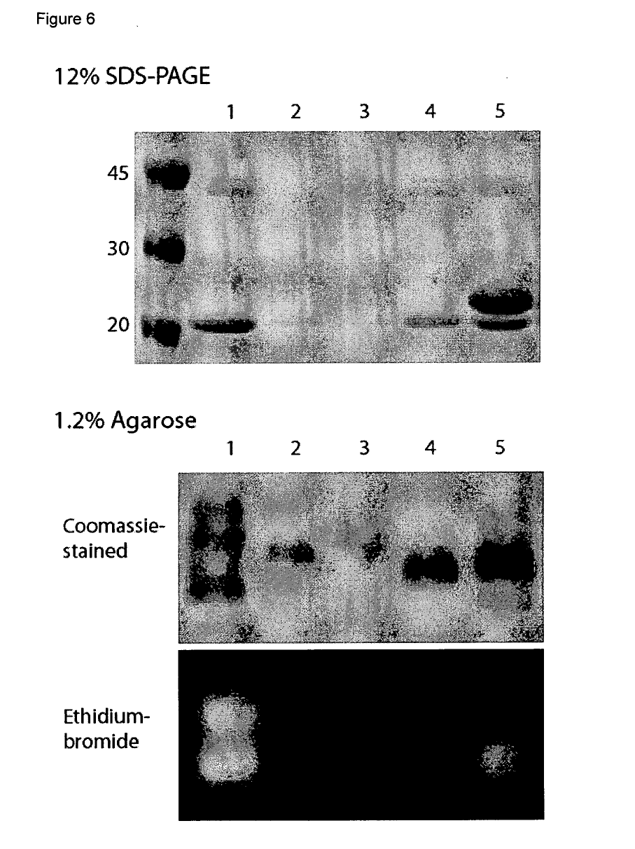

SDS-PAGE electrophoresis (FIG. 6, upper panel) showed that the VLPs resulting from the co-infiltration of leaves with pEAQ-HT-VP60 and pEAQ-HT-24K (lane 3) had a coat protein composition similar to that of either a natural mixture CPMV particles or purified Top component isolated from plants (lanes 1 and 4) and to VLPs produced in insect cells (lane 2). The only significant difference was the presence of larger amounts of the unprocessed form of the S protein in the VLPs produced by the co-infiltration than in the plant- or insect cell-derived particles. This may simply reflect the relative age of the preparations, the slower migrating form of the S protein is converted to the faster form on storage.

As an alternative to using pEAQ-HT-24K to process VP60, we investigated whether it is possible to achieve processing with a full-length version of RNA-1. To this end, pEAQ-HT-VP60 was co-infiltrated with pBinP-S1-NT and potential VLPs isolated. SDS-PAGE electrophoresis of these VLPs showed that they contained mature L and S proteins (FIG. 6, Top panel, lane 5), indicating the RNA-1 can catalyse effective processing of VP60 in plants.

Gel electrophoresis of CPMV particles on non-denaturing agarose gels has previously shown to be an effective method for distinguishing between empty and RNA-containing particles, the migration of RNA-containing particles being greater than that of empty particles (Steinmetz et al., 2007). However, the migration of the particles is not only dependent upon their RNA content but also upon the presence or absence of the 24 carboxyl-terminal amino acids of the S protein which is often lost by proteolysis. FIG. 6 (lower panels) show an agarose gel stained with either Coomassie blue (top) which is specific for proteins or with ethidium bromide to detect nucleic acids. The pattern of bands resulting from electrophoresis of a natural mixture of particles isolated from infected plants can be revealed by staining with either Coomassie blue or ethidium bromide, indicating that they contain both protein and nucleic acid. By contrast, the particles resulting from cleavage of VP60 by the 24K proteinase either in insect cells (lane 2) or in plants (lane 3) can be seen only with Coomassie blue staining, a situation identical to that found with purified Top components (lane 4). These results are consistent with particles being empty (nucleic acid-free). Intriguingly, VLPs isolated from leaves co-infiltrated with pEAQ-HT-VP60 and pBinP-S1-NT gave rise to two bands on the agarose gel, the slower migrating of which stained only Coomassie blue, while the faster stained with both Coomassie blue and ethidium bromide. This suggests that the slower band consists of nucleic acid-free particles while faster one while the faster one has encapsidated nucleic acid, probably the RNA-1 generated by pBinP-S1-NT.

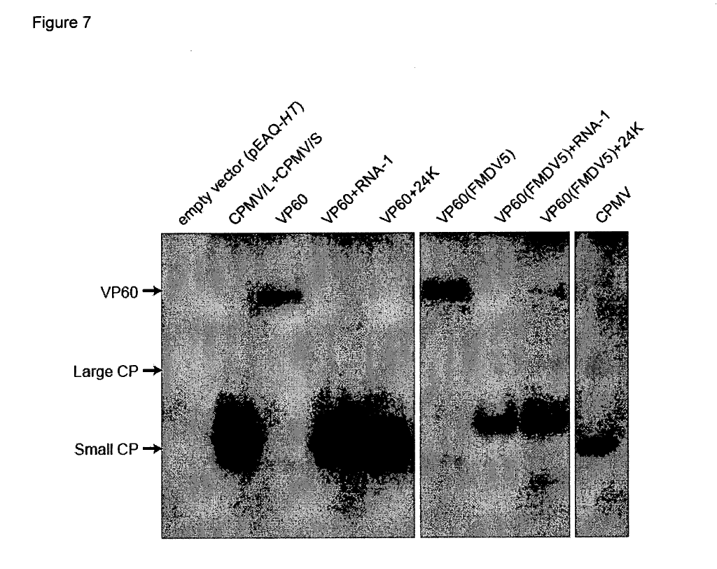

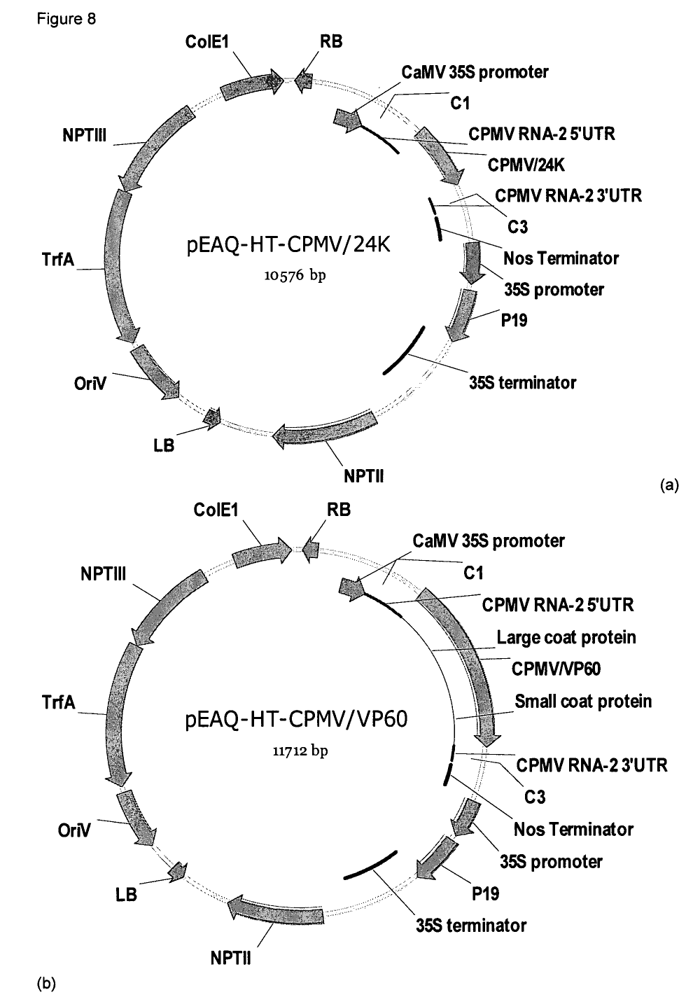

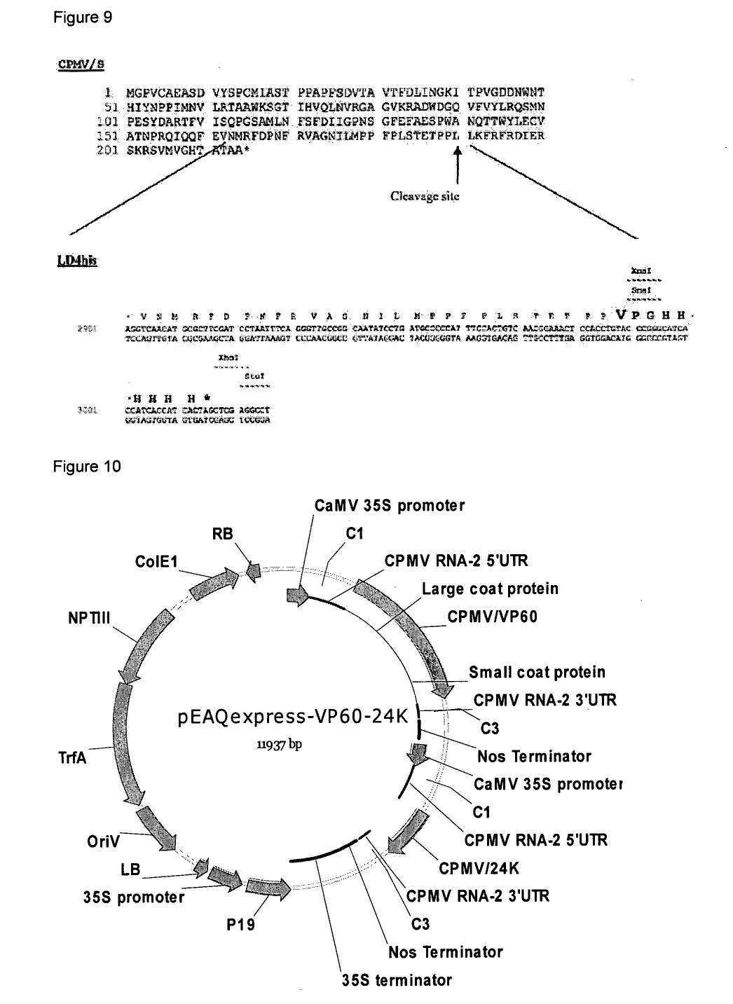

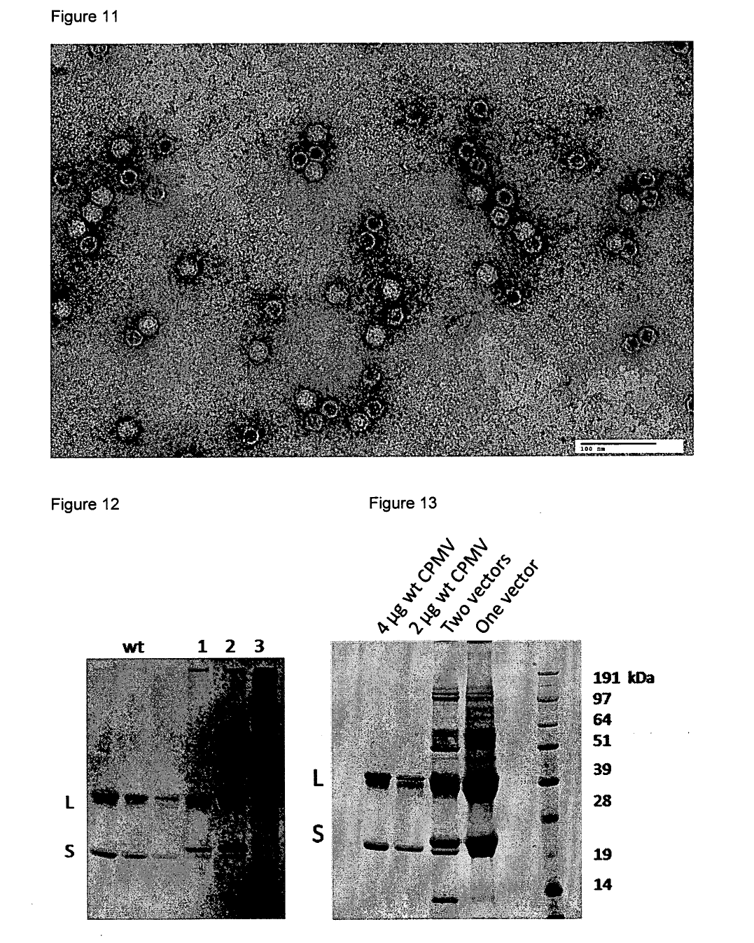

FIG. 7 is a Western blot showing the processing of VP60 in plants by the 24 kDa proteinase, including a demonstration that VP60 can be modified such that the S coat protein includes a 19 amino acid FMDV sequence inserted in 13B-13C loop, without impairing proteolytic processing.

### Example 5

### Discussion of Examples 1 to 4

The Examples above demonstrate the first report of the generation of CPMV capsids via proteolytic processing.

Insect cells have only previously been shown to support both the activity of the 24K proteinase in cis (van Bokhoven et al., 1990;1992) and the formation of VLPs from the individually expressed L and s proteins (Shanks and Lomonossoff, 2000). When full-length version RNA-2-encoded polyproteins were used as the coat protein precursors in the above Examples, the mature L and S proteins were released only when an RNA-1 construct encoding both the 32K proteinase co-factor and the 24K proteinase was used to achieve processing. This observation is consistent with the conclusion from in vitro translation studies that both the 32K and 24K proteins are required for processing the RNA-2-encoded polyproteins at the 58K/48K-L junction (Vos et al., 1988).

However, we have further been able to demonstrate that the L and S proteins produced by processing of the full-length RNA-2 polyproteins can assemble into VLPs, the first time this has been observed.

By contrast to the situation when the full-length RNA-2 polyproteins were used, co-expression of the 24K proteinase alone was sufficient to achieve processing of VP60 into the L and S proteins. This is consistent with previous studies that the 24K proteinase alone could cleave at the L-S junction to release the S protein when the proteinase and VP60 sequences were part of the same artificial precursor (Garcia et al., 1987; Vos et al., 1988; Wellink et al., 1996). However, prior to this report no direct processing of VP60 by the 24K proteinase to give the mature L and S protein had previously been observed. The fact that release of the L and S proteins from VP60 by the action of the 24K proteinase in trans also leads to the formation of VLPs demonstrates that VP60 can act as a coat protein precursor as originally proposed by Franssen et al. (1982).

The relevance of VP60 cleavage to capsid formation in planta was confirmed by the demonstration that the transient co-expression of VP60 and the 24K proteinase in N. benthamiana leaves lead to the production of the L and S proteins and formation of capsids.

Sucrose gradient density analysis of the VLPs produced by proteolytic processing in insect cells suggested that the particles are essentially RNA-free as they sediment to a position characteristic of Top components produced during a natural infection. In the case of extracts from cells expressing bv-VP60/24K, which produced the largest amount of VLPs, this observation was confirmed by agarose gel electrophoresis of particles. The observation that only the fast migrating form of the S protein is generated through co-expression of bv-2 and bv-1A or by expression of bv-VP60/24K while cells co-infected with bv-VP60 and bv-1A generate both the fast and slow migrating forms of the S protein is unclear. Expression of VP60 in the absence of the 24K proteinase does not lead to VLP formation, a result consistent with that of Nida et al. (1992). However, the protein appears to form amorphous aggregates which migrate over a considerable portion of a sucrose density gradient. Analysis of the fractions from the gradients revealed a protein of approximately 175 kDa which is roughly 3 times the molecular weight of VP60. This is consistent with it being an SDS-stable trimer of VP60 which might represent an intermediate in the VLP assembly pathway—assembly to produce capsids only proceeding after cleavage at the L-S site. This raises the possibility that capsid assembly starts by the association of VP60 molecules around the 3-fold axes which in the mature particles are occupied by the L protein

A further interesting feature of the expression of VP60 in both insect cells and in plants is the appearance, in the absence of the 24K proteinase, of low amounts of protein whose size is identical to the fast form of the S protein. This product most likely arises through the non-specific cleavage of the linker between the C-terminal domain of the L protein and the S protein. This linker consists of 25 amino acids and is probably in an extended conformation making it susceptible to cleavage (Clark et al., 1999).

### Example 6

### Presence of VP60 and 24K Genes in the Same T-DNA Region Enhances eVLP Yield

FIG. 10 shows the structure of a combined high-level expression plasmid used for plant expression (pEAQexpress-VP60-24K). The complete sequence is provided as SEQ ID NO 3.

As shown in FIG. 13, expression can be enhanced at least three-fold if one vector encodes both genes, as compared with the use of two separate vectors.

In conjunction with the improved protocol described in Example 7, yields of up to 0.2 g/Kg leaf tissue (i.e. 0.02% w/w) or more can be achieved.

### Example 7

### Improved Extraction of Cowpea Mosaic Virus Empty Virus-Like Particles

The method for extraction of CPMV eVLPs from N. benthamiana was initially based on a protocol from van Kammen and de Jager (Database of plant viruses, 1971).

Since the 1971 protocol was originally designed for wild-type particles from cowpea, it was optimised for eVLPs. To identify the key steps in the extraction process where particles were being lost, samples were collected from each stage of the extraction and analysed by SDS-PAGE and western blots. Based on this, the protocol was modified and validated by analysing samples from each step again.

The following observations were made and the eVLP extraction protocol was modified accordingly.

| OLD PROTOCOL | PROBLEM | MODIFIED PROTOCOL |
|---|---|---|
| Leaf tissue was | eVLPs degrade upon | Leaf tissue is processed |
| harvested and | freezing. | fresh (e.g. in cold room). |
| frozen. | | |
| Sodium phosphate | Polysaccharides from N. | 2% PVPP (polyvinyl- |
| buffer was used. | benthamiana purify | polypyrrolidone) is used |
| | along with eVLPs and | while grinding plant |
| | form a sticky pellet after | tissue as it binds to |
| | ultra-centrifugation. | polysaccharides and |
| | | phenolics from the plant. |
| | | Since PVPP is insoluble, |
| | | it is separated in the first |
| | | spin and doesn't affect |
| | | the next steps. |
| A 1:1 chloroform- | Over a 50% of the eVLPs | This step is deleted |
| butanol mixture | were degrading at this | completely. Further |
| was used to remove | step. | purification steps are |
| chlorophyll and | | done on the final sample |
| other plant proteins | | to remove impurities. |
| from the extract. | | |
| A 27000 g spin is | eVLPs were being lost | After adding buffer to |
| done straight after | in the pellet after the | the PEG precipitate, |
| PEG precipitation. | 27000 g spin. This | it is resuspended |
| | indicates that the PEG | thoroughly by vortexing, |
| | ppt. was not resuspended | pippeting up and down |
| | properly prior to the spin. | and shaking the tubes |
| | | vigorously for 2-3 hours. |
| Ultra-centrifugation | The sedimentation | The centrifugation spin |
| done for 2:15 hours. | coefficient of eVLPs | time is increased to 2:30 |
| | (58 S) is lesser than that | hours. |
| | of the wt particles (118 S). | |

A preferred modified protocol is as follows:

### Equipment

Electric blender, Centrifuge, Ultracentrifuge, Magnetic stirrer, Vortex mixer

### Procedure

1. Harvest infiltrated leaves and homogenise leaf tissue with 3 volumes (for 1 g tissue, use 3 mls) of 0.1M Sodium phosphate buffer, pH=7.0 using a blender.
2. Add Polyvinyl-polypyrrolidone (PVPP) to the buffer to a final concentration of 2%. PVPP binds to contaminating polysaccharides and phenolics from the plant.
3. Squeeze homogenate through two layers of muslin cloth and spin at 13000 g for 20 mins at 4° C. to remove cell debris.
4. To the supernatant, add polyethylene glycol 6000 (PEG 6000) to a final concentration of 4% and NaCl to 0.2 M. Stir at 4° C. overnight to precipitate the virus particles.
5. Spin at 13000 g for 20 mins at 4° C. to pellet the PEG precipitate.
6. Dissolve the pellet in 0.01 M sodium phosphate buffer, pH=7 (0.5 ml/g leaf tissue) and resuspend thoroughly by vortexing.
7. Spin at 27000 g for 20 mins at 4° C.
8. Transfer the supernatant to ultracentrifuge tubes and spin at 118,700 g for 150 mins at 4° C. in an ultracentrifuge.
9. Resuspend pellet in a small volume (by way of non-limiting example, 500 μl) of buffer and spin at 10,000 g for 5 mins on a bench-top centrifuge to remove possible contaminants.

The supernatant contains purified CPMV eVLPs.

As shown in FIG. 11, using the modified protocol and the new construct, the yield of eVLPs from N. benthamiana is in excess of 0.2 g/kg FWT. This is about 10-fold more than what it was before optimisation. The eVLPs produced in this way are about 30 nm in size.

As shown in FIG. 12, removal of the organic extraction step increases eVLP recovery The L and S proteins from particles have been separated by SDS-PAGE using 12% NuPAGE gels stained with Instant Blue Coomassie stain. Comparison of Lanes 1 and 2 shows that deletion of the organic clarification step (Lane 2) increases recovery by about 60%. An increase in contaminants is seen but these can be easily removed using dialysis and desalting columns.

### Example 8

### Cowpea Mosaic Virus Unmodified Empty Virus-Like Particles can be Loaded with Metal and Metal Oxide

The wild-type virus CPMV capsid is stable to moderately high temperature, for example 60° C. (pH 7) for at least one hour, across the range of pH 4-10, and in some organic solvent-water mixtures. This degree of stability is extremely valuable as it enables the particles to be chemically modified. For example, amino acid residues on the solvent-exposed capsid surface can be used to selectively attach moieties such as redox-active molecules, fluorescent dyes, metallic and semi-conducting nanoparticles, carbohydrates, DNA, proteins and antibodies.^{4,8,9} As well as chemical modification, the availability of infectious cDNA clones has allowed the production of chimeric virus particles presenting multiple copies of peptides on the virus surface.²² One application of chimeric virus has been to produce externally mineralized virus-templated monodisperse nanoparticles.^{23,24} However, for many purposes, such as targeted magnetic field hyperthermia therapy, it would be desirable to produce particles that are internally mineralized; eVLPs offer a route to how this could be achieved.

The method for the production of eVLPs in this example used pEAQ-HT system to simultaneously express the VP60 coat protein precursor and the 24K proteinase in plants via agro-infiltration. As described above, efficient processing of VP60 to the L and S proteins occurred, leading to the formation of capsids which were shown to be devoid of RNA.

Incubation of CPMV eVLPs, suspended in 10 mM sodium phosphate buffer pH 7, with cobalt chloride solution, followed by washing, and then subsequent reduction with sodium borohydride gave cobalt-loaded VLPs (cobalt-VLPs) in which cobalt is encapsulated within the capsid core. Recovery of cobalt-VLPs is approximately 70% based on initial CPMV eVLP concentration. An unstained transmission electron microscopy (TEM) image clearly showed the cobalt core (not shown) and energy dispersive X-ray spectroscopy (EDXS) confirmed the presence of cobalt. CPMV eVLPs, prior to the reaction, were not visible in the TEM without staining. A uranyl acetate negatively stained TEM image of cobalt-VLPs showed the intact VLP protein shell (the capsid) surrounding the metallic core (not shown). Dynamic light scattering (DLS) of the particles in buffer confirms that the external diameter of the VLPs (31.9±2.0 nm compared to 32.0±2.0 nm for CPMV eVLP) does not change significantly on internalization of cobalt and that the particles remain monodisperse. The cobalt particle size of ca. 26 nm is as expected if the interior cavity of the VLP is fully filled.

A similar approach was employed to generate internalized iron oxide. A suspension of CPMV eVLPs was treated with a mixture of ferric and ferrous sulfate solutions in a molar ratio of 2:1, under conditions which favor the formation of Fe₃O₄, magnetite. After mixing overnight at pH 5.1, the particles were washed on 100 kDa cut-off columns before the pH was raised to 10.1. The resultant iron oxide-VLPs were purified and obtained in 40-45% yield based on initial CPMV eVLP concentration. Again, unstained TEM images clearly showed the metal oxide core; negatively stained TEM images showed the external capsid protein; EDXS confirms the presence of iron and oxygen; and DLS shows that the particles are monodisperse with an external diameter (˜31.6±2.0 nm) changed little compared to CPMV eVLPs. The zeta potentials for suspensions of eVLPs (−32.0±2.3 mV) cobalt-VLPs (−32.9±1.8 mV) and iron oxide-VLPs (−32.1±2.4 mV) indicate that the colloids have good stability and show little propensity to aggregate. In each case, control experiments performed under identical conditions except for the absence of eVLPs gave non-specific bulk precipitation with a wide size distribution of nanoparticles as observed by TEM and DLS; thus the eVLPs are essential for controlled nanoparticle growth.

Previously, we have found that externally mineralized, for example silicated, CPMV particles are robust and the coat proteins cannot be released by denaturation under harsh conditions (e.g. denaturing with sodium dodecyl sulfate at 100° C. for 30 min).²³ Here, however, sodium dodecyl sulfate polyacrylamide gel electrophoresis (SDS-PAGE) of the denatured proteins from wild-type CPMV isolated from infected plants, “top component” consisting of empty particles from a wild-type infection,²⁵ CPMV eVLPs, cobalt-VLPs and iron oxide-VLPs (not shown) all gave a similar pattern of bands after Coomassie Blue staining; the slower running L protein and faster running forms of the S protein. The difference in the S proteins isolated from wild-type virus and eVLP samples is due to the differing degrees of C-terminal processing. These results indicate that the coat proteins are accessible and that the mineralization is internal. Further confirmation that the capsid structure is preserved, and that the eVLPs were not externally mineralized, was provided by analysis of the intact particles by agarose gel electrophoresis. Coomassie Blue staining revealed that all the VLPs which were devoid of RNA, whether containing internalized metal/metal oxide or not, had the same mobility. By contrast the RNA-containing particles from natural populations of particles gave a typical complex pattern.

Further analysis confirmed that the mineralized VLPs contained both the coat proteins and either cobalt or iron, respectively. Samples of each of unmineralized eVLP, cobalt-VLP and iron oxide-VLP were spotted onto a nitrocellulose membrane which, after blocking, was probed with polyclonal antibodies raised in rabbits against CPMV particles. The binding of the antibodies was detected using a goat anti-rabbit IgG coupled to horseradish peroxidise and the signals were visualized by electrochemiluminescence. In each case a dark signal was obtained confirming the presence of CPMV coat protein in all the VLP samples (not shown). Similarly, each of eVLP, cobalt-VLP and iron oxide-VLP were spotted onto a nitrocellulose membrane and probed with either a cobalt-specific stain (1-nitroso-2-naphthol) or Prussian blue staining to identify iron. Only the cobalt-VLP stained orange, showing the presence of cobalt, and only the iron oxide-VLP stained blue, showing the presence of iron, within the VLPs.

To demonstrate that the external coat of the metal containing VLPs is still amenable to chemical modification, cobalt-VLPs were functionalized at solvent-exposed lysines with succinimide ester activated biotin by an adaptation of our standard procedure.²⁶ The binding of both biotinylated-cobalt-VLPs and biotinylated-eVLPs to streptavidin-modified chips was monitored by surface plasmon resonance. In each case a response was observed, confirming that chemical modification of the VLP capsid exterior had successfully occurred, irrespective of the internal mineralization. This provides the first evidence that the external surface of eVLPs and internally mineralized VLPs can be chemically modified using the same approach taken for wild-type CPMV. Comparison of normalized sensograms recorded at the same VLP concentration (based on protein content as estimated by UV-visible spectroscopy) showed a two and a half fold increase in resonance units consistent with the increase in mass associated with the loading of cobalt within the VLP.

In conclusion, this Example confirms that CPMV eVLPs can, without further genetic or chemical modification, easily encapsulate inorganic payloads such as cobalt or iron oxide within the capsid interior. Previously, it has been shown that wild-type CPMV particles are permeable to cesium ions and that penetration probably occurs via channels at the five-fold axes of the virus particles, where the S subunits cluster. These channels are funnel-shaped, with the narrow end at the outer surface of the virus particle and the wider end in the interior.¹⁹ The opening at the narrow end is about 7.5 Å in diameter. Further down the five-fold axis, a second constriction can be found which occurs as a result of the three N-terminal residues of the S subunits forming a pentameric annulus structure. In this structure, the amino group of the N-terminus forms a hydrogen bond with the main chain carbonyl oxygen of the neighbouring third residue; the opening at this point is ca. 8.5 Å. We propose that it is through these channels that the cobalt and iron ions enter the inside of the eVLP. That the pentameric annulus controls access to the interior of the eVLPs is supported by the observation that the addition of a methionine residue to the N-terminus of the S protein prevents penetration by cobalt ions, presumably by occluding the channel with a bulky side chain.²⁷ The charge on the internal surface of the capsid is negative, arising from glutamic acid and aspartic acid residues. The electrostatic interactions between the internal surface and the incorporated metal ions entrap them within the capsid. Even six hours dialysis against buffer does not remove the electrostatically entrapped metal ions. On further treatment, either reduction for cobalt or alkaline hydrolysis for the iron oxide, the metal ions act as nucleation sites for metal particle formation or further autocatalytic hydrolysis²⁸ to produce iron oxide, respectively.

The encapsulation processes occur at ambient temperature, in aqueous media, producing little waste, so are environmentally friendly. In addition, amino acid residues on the exterior surface of the internally mineralized particles remain amenable for chemical modification. The ability to both encapsulate materials (e.g. nanoparticles or drugs) within the eVLP and to chemically modify the external surface, opens up routes for the further development of CPMV-based systems for the targeted delivery of therapeutic agents and for other uses in biomedicine.

### Example 9

### Cowpea Mosaic Virus Unmodified Empty Virus-Like Particles can be Loaded with Dyes and Drugs

Two compounds were selected: rhodamine (a fluorescent dye) and doxorubicin (a fluorescent drug). Both compounds were theoretically just small enough to enter eVLPs through the pores at the 5-fold axes.

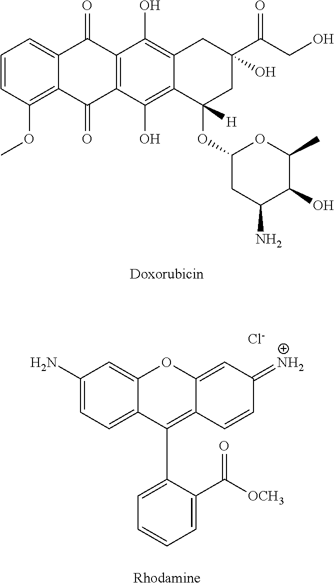

The method for the production of eVLPs in this example used a solution of 1 mg/ml eVLP mixed with a final concentration of 1 mg/ml Doxorubicin or Rhodamine and incubated overnight at 4C with occasional agitation.

eVLPs were concentrated and washed with water to remove unbound drug/dye.

The loaded eVLPs were coated with the positively polymer polyallylamine hydrochloride (PAH) to coat the virus and prevent leaching of the drug/dye.

Particles were washed with water.

Examination of loaded eVLPs on agarose gels showed co-migration of coat protein and fluorescence.

Uv/vis spectrophotometry suggests 8 Rhodamine or 10 Doxorubicin molecules per eVLP.

Gemcitabine is a nucleoside analog used in chemotherapy. It is marketed as Gemzar by Eli Lilly and Company. It is predicted to be smaller than either of the compounds above may be loaded into eVLPs using corresponding methods.

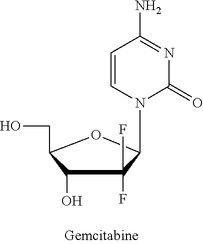

### Example 10

### Oligonucleotides for Cloning of Sequences

a) 24K Cloning 5′ oligo

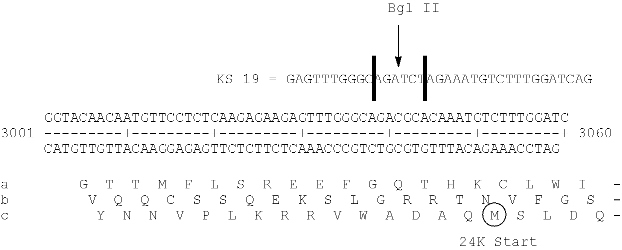

KS 19 = GAGTTTGGGCAGATCTAGAAATGTCTTTGGATCAG

b) 24K Cloning 3′ oligo

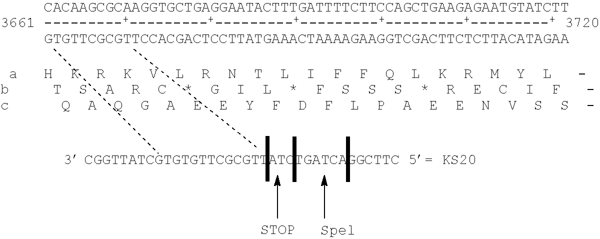

KS 20 = CTTCGGACTAGTCTATTGCGCTTGTGCTATTGGC

c) VP60 Cloning 5′ oligo

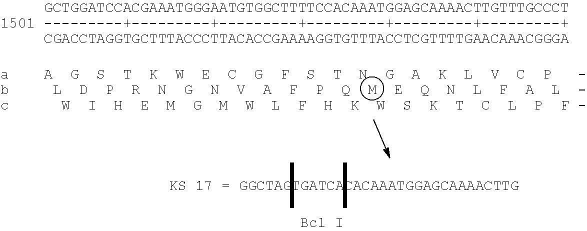

KS 17 = GGCTAGTGATCACACAAATGGAGCAAAACTTG

d) VP60 Cloning 3′ oligo

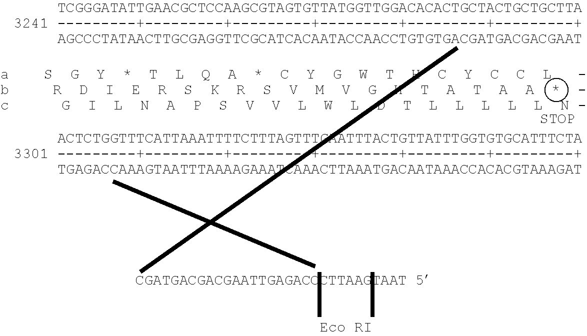

KS 18 = TAATGAATTCCCAGAGTTAAGCAGCAGTAGC

e) Cloning of 1A-5′ oligo

Into Bam HI compatible site (Bbs I) using Bam Hi site in RNA 1 at 3857 and—

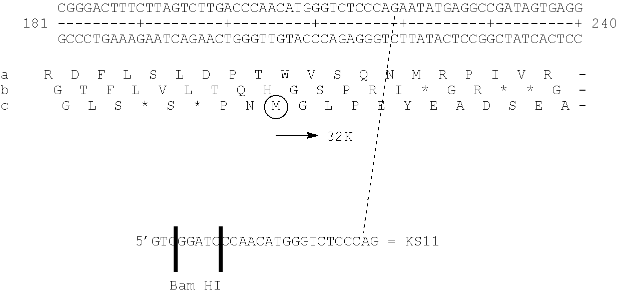

KS11 = GTCGGATCCCAACATGGGTCTCCCAG

f) Cloning of 1A-3′ oligo

Into Bam HI compatible site (Bbs I) using Bam Hi site in RNA 1 at 3857

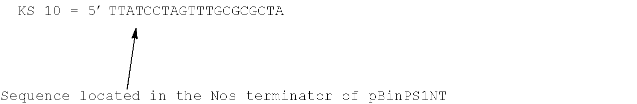

After PCR the product was digested with Bam HI and the appropriate product ligated into pMFBD previously digested with Bam HI

KS 10 = 5′ TTATCCTAGTTTGCGCGCTA

g) 24K protease sequence map

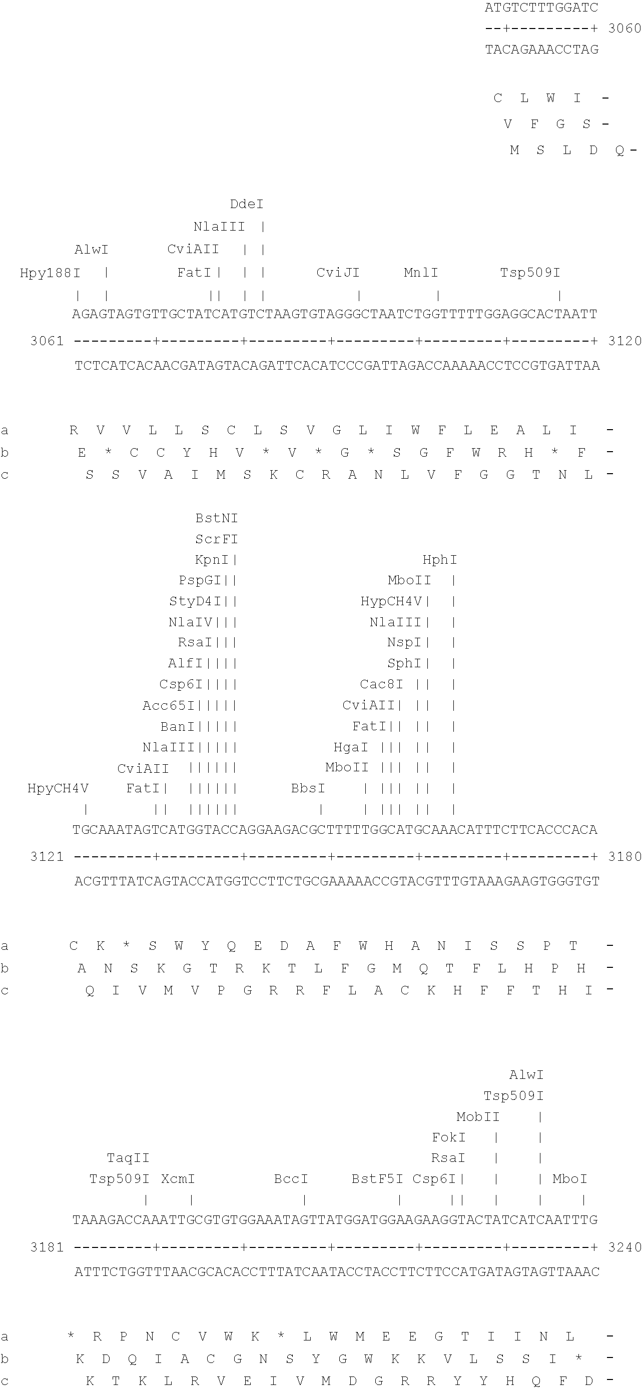

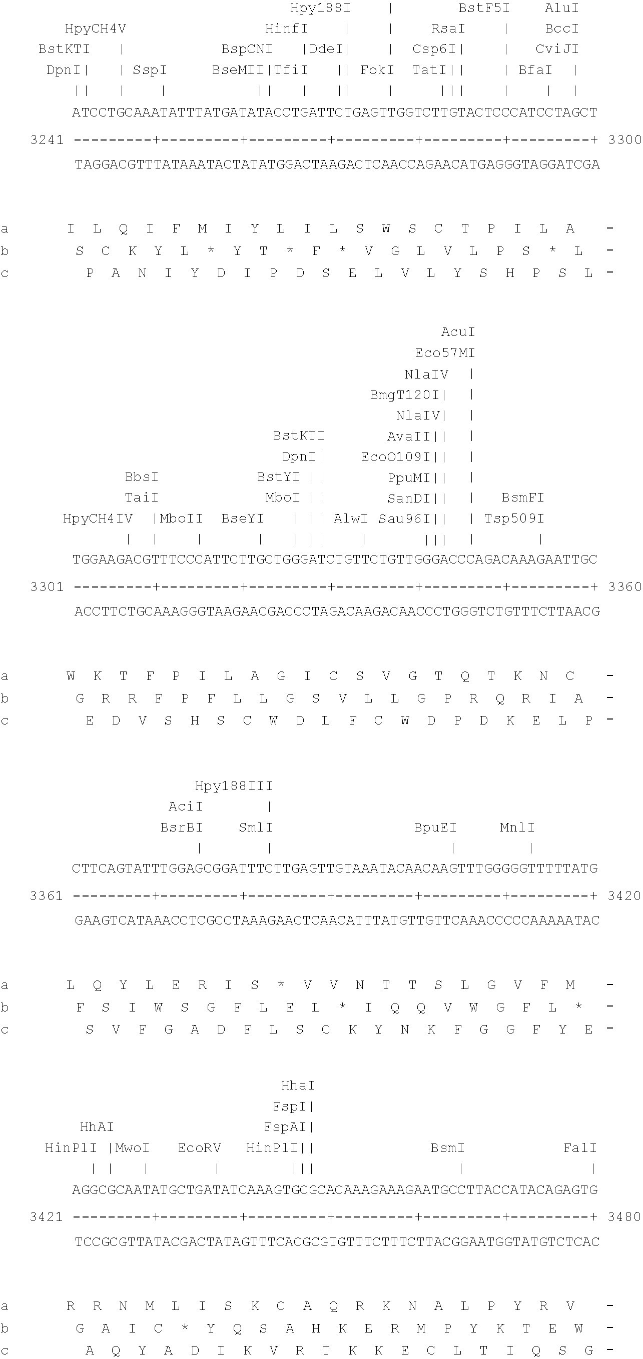

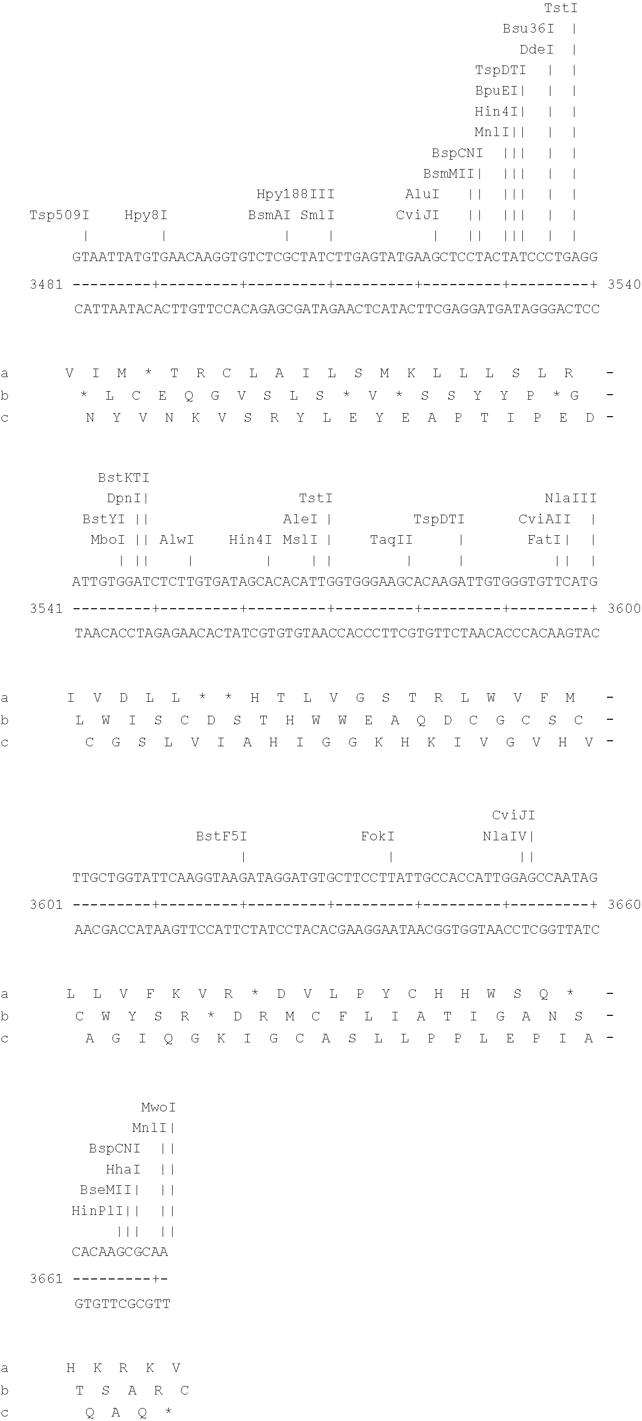

h) VP60 sequence map

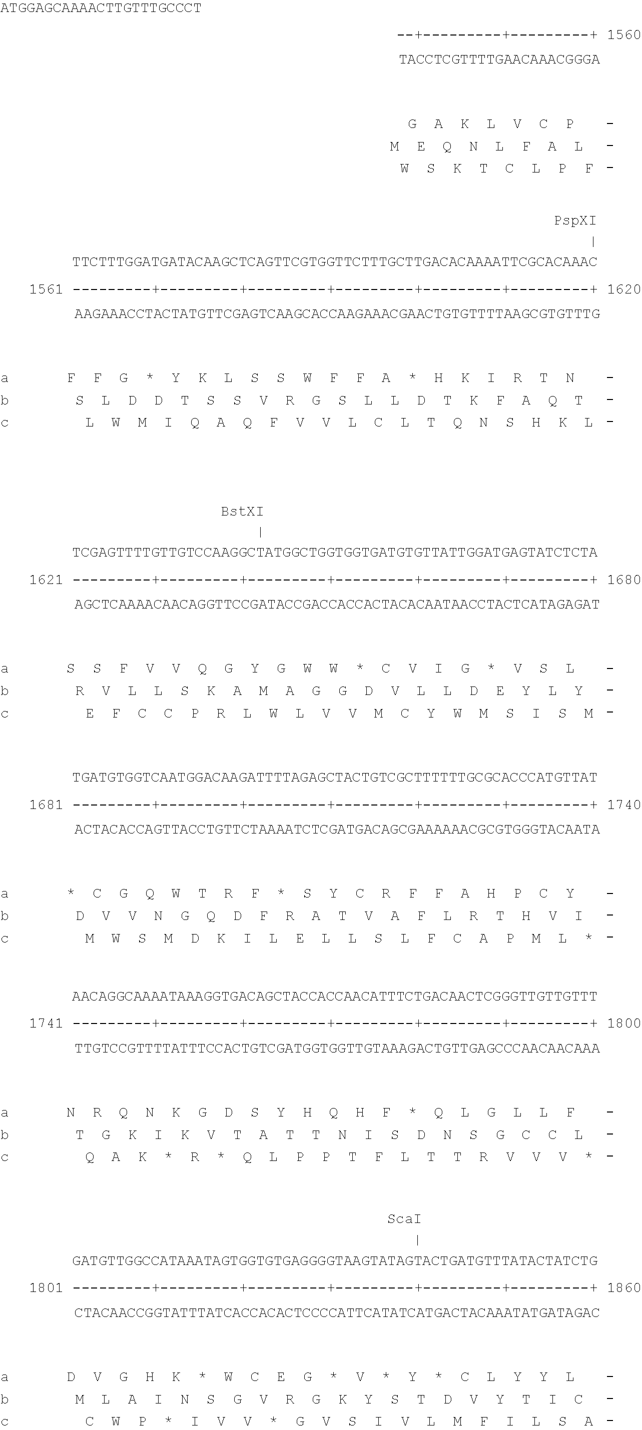

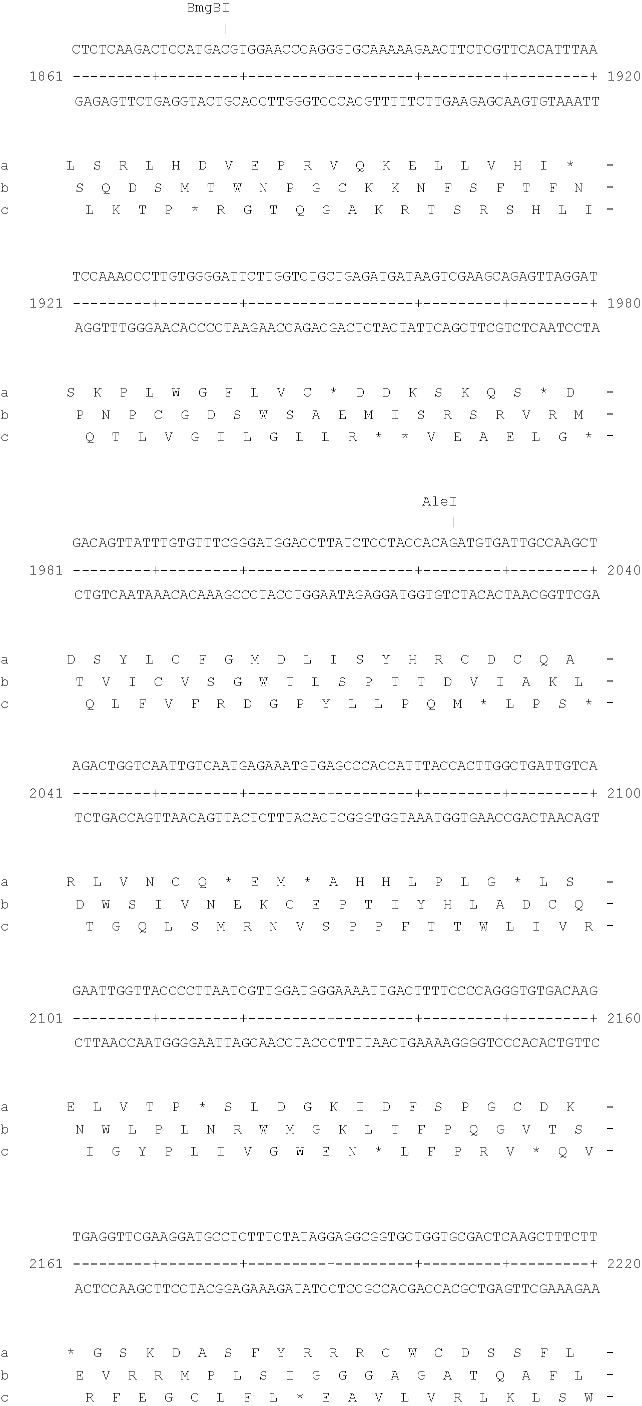

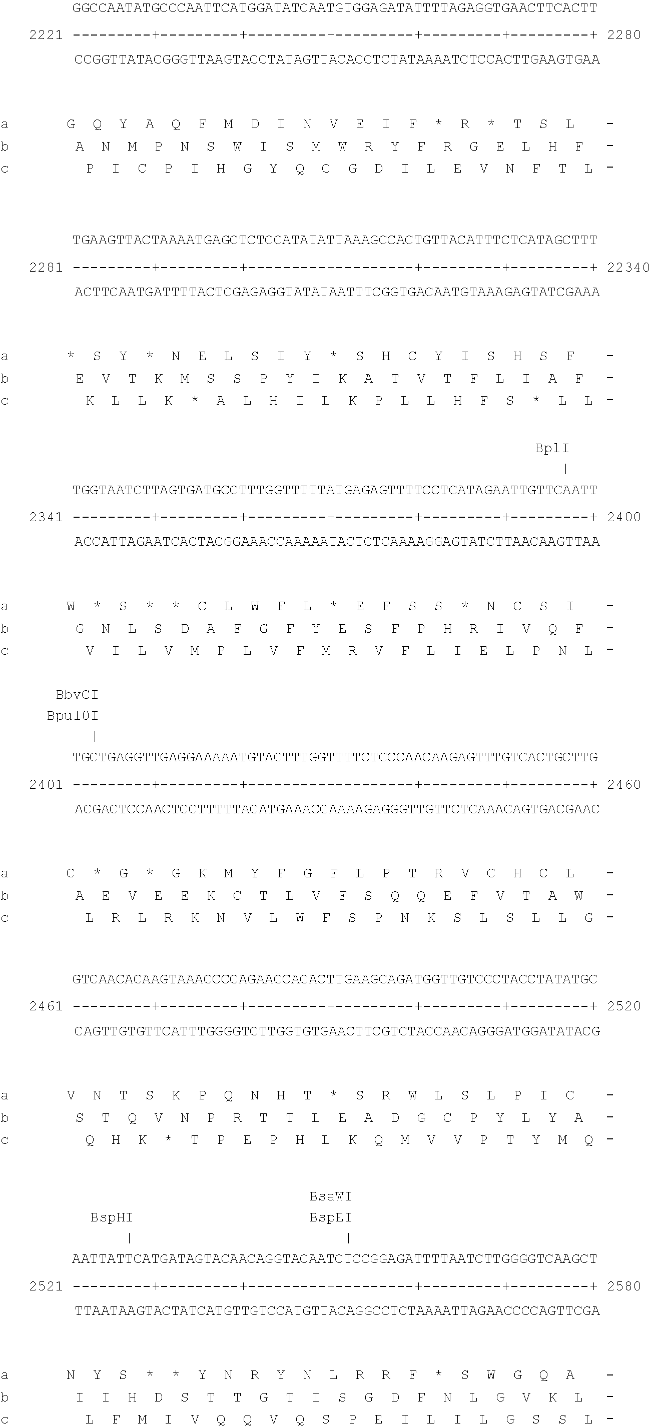

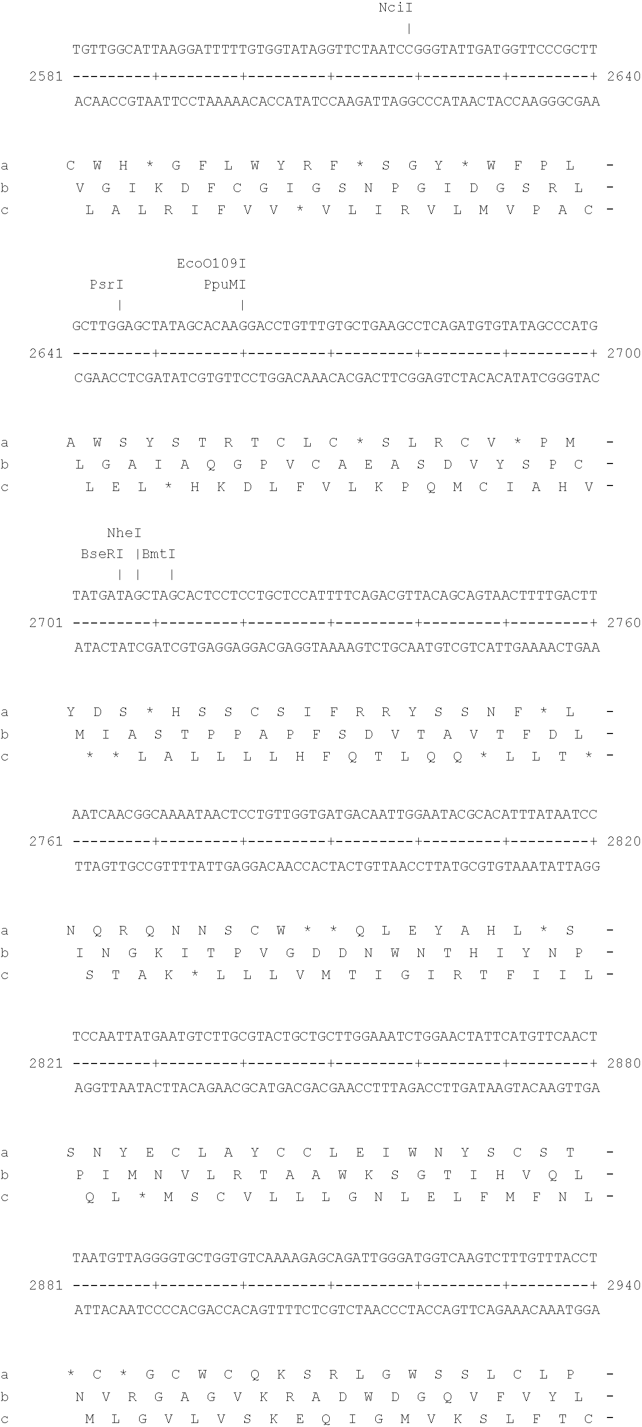

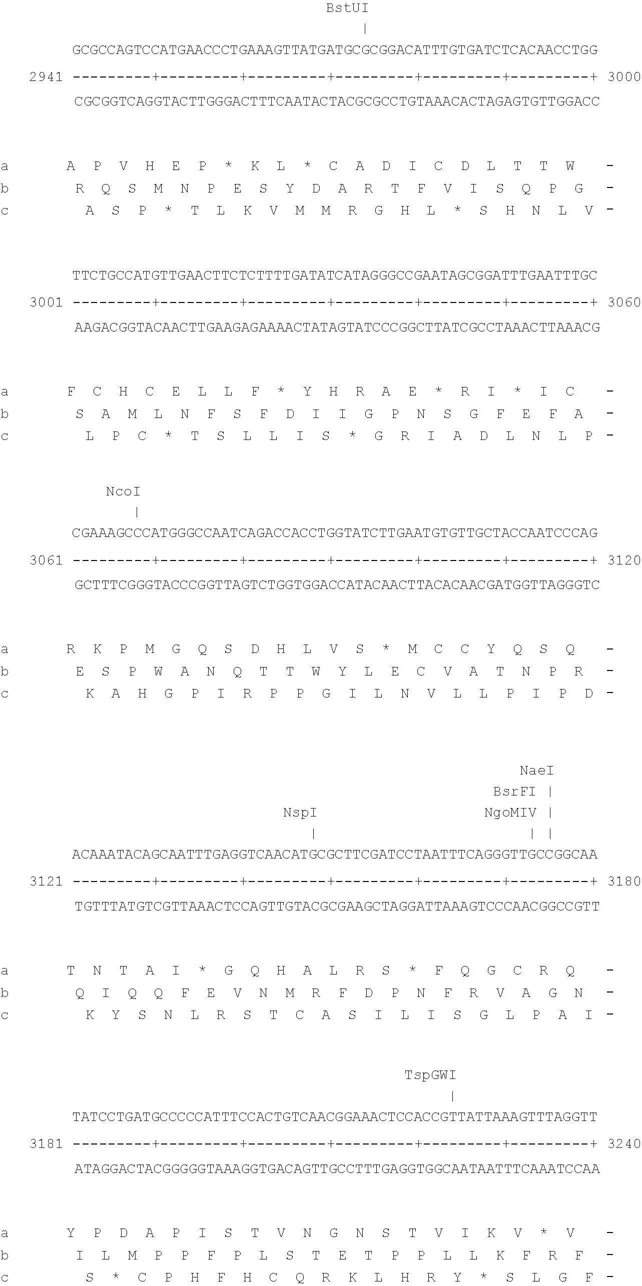

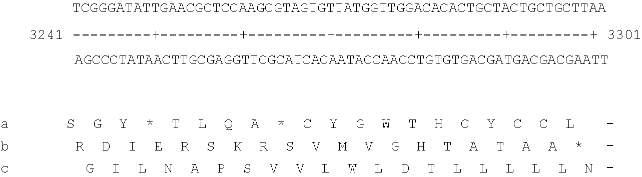

### REFERENCES

(1) Flynn, C. E.; Lee, S.-W.; Peelle, B. R., Belcher, A. M. Acta Mat. 2003, 51, 5867-5880.
(2) Singh, P.; Gonzalez, M. J.; Manchester, M. Drug Develop. Res. 2006, 67, 23-41.
(3) Uchida, M.; Klem, M. T.; Allen, M.; Suci, P.; Flenniken, M.; Gillitzer, E.; Varpness, Z.; Liepold, L. O.; Young, M.; Douglas, T. Adv. Mater. 2007, 19, 1025-1042.
(4) Steinmetz, N. F.; Evans, D. J. Org. Biomol. Chem. 2007, 5, 2891-2902.
(5) Young, M.; Willits, D; Uchida, M.; Douglas, T. Annu. Rev. Phytopathol. 2008, 46, 361-384.
(6) Evans, D. J. J. Mater. Chem. 2008, 18, 3746-3754,
(7) Escosura, A. de la; Nolte, R. J. M.; Cornelissen, J. J. L. M. J. Mater. Chem. 2009, 19, 2274-2278.
(8) Evans, D. J. Biochem. Soc. Trans. 2009, 37, 665-670.
(9) Manchester, M.; Steinmetz, N. F. (Eds) Curr. Top. Microbiol. Immunol.; Viruses and Nanotechnology; Springer-Verlag: Berlin, Heidelberg, 2009.
(10) Douglas, T.; Young, M. Nature 1998, 393, 152-155.
(11) Douglas, T.; Young, M. Adv. Mater. 1999, 11, 679-681.
(12) Klem, M. T.; Young, M.; Douglas, T. J. Mater. Chem. 2008, 18, 3821-3823.
(13) Escosura, A. de la; Verwegen, M.; Sikkema, F. D.; Comellas-Aragones, M.; Kirilyuk, A.; Rasing, T.; Nolte, R. J. M.; Cornelissen, J. J. L. M. Chem. Commun. 2008, 1542-1544.
(14) Langeveld, J. P. M.; Brennan, F. R.; Martinez-Torrecuadrada, J. L.; Jones, T. D.; Boshuizen, R. S.; Vela, C.; Casal J. I.; Kamstrup, S.; Dalsgaard, K.; Meloen, R. H.; Bendig, M. M.; Hamilton, W. D. O. Vaccine 2001, 19, 3661-3670.
(15) Rae, C.; Koudelka, K. J.; Destito, G.; Estrada, M. N.; Gonzales, M. J.; Manchester, M. PLoS ONE 2008 3(10):e3315.doi:10.1371/journal.pone.0003315.
(16) Phelps, J. P.; Dang, N.; Rasochova, L.; J. Virol. Meth. 2007, 141, 146-153.
(17) Ochoa, W. F.; Chatterji, A.; Lin, T.; Johnson, J. E. Chemistry & Biology 2006, 13, 771-778.
(18) Saunders, K.; Sainsbury, F.; Lomonossoff, G. P. Virology 2009, 393, 329-337.
(19) Lin, T; Johnson, J. E. Adv Virus Res 2003, 62, 167-239.
(20) Sainsbury, F.; Lomonossoff, G. P. Plant Physiol. 2008, 148, 1212-1218.
(21) Sainsbury, F.; Thuenemann, E. C.; Lomonossoff, G. P. Plant Biotech J. 2009, 7, 682-693.
(22) Lomonossoff, G. P.; Hamilton, W. D. O. Curr. Top. Microbiol. Immunol. 1999, 240, 177-189.
(23) Steinmetz, N. F.; Shah, S, N.; Barclay, J. E.; Rallapalli, G.; Lomonossoff, G. P.; Evans, D. J. Small 2009, 5, 813-816.
(24) Shah, S, N.; Steinmetz, N. F.; Aljabali, A. A. A.; Lomonossoff, G. P.; Evans, D. J. Dalton Trans. 2009, 8479-8480.
(25) Lomonossoff, G. P.; Johnson, J. E. Prog. Biophys. Mol. Biol. 1991, 55, 107-137.
(26) Steinmetz, N. F.; Calder, G.; Lomonossoff, G. P.; Evans, D. J. Langmuir 2006, 22, 10032-10037.
(27) Aljabali, A. A. A.; Sainsbury, F.; Evans, D. J.; Lomonossoff, G. P. unpublished results.
(28) Wade, V. J.; Levi, S.; Arosio, P.; Treffry, A.; Harrison, P. M.; Mann, S. J. Mol. Biol. 1991, 221, 1443-1452.

### OTHER REFERENCES

Bu, M. and Shih, D. S. (1989). Inhibition of proteolytic processing of the polyproteins of cowpea mosaic virus by hemin. Virology 173, 348-351.
Chatterji, A., Ochoa, W., Paine, M., Ratna, B. R., Johnson, J. E. & Lin, T. (2004). New addresses on an addressable virus nanoblock Uniquely reactive Lys residues on cowpea mosaic virus. Chem. Biol. 11, 855-863.
Clark, A. J., Bertens, P., Wellink, J., Shanks, M. & Lomonossoff, G. P. (1999). Studies on hybrid comoviruses reveal the importance of three-dimensional structure for processing of the viral coat proteins and show that the specificity of cleavage is greater in trans than in cis. Virology 262, 184-194.
Destito, G., Schneemann, A. and Manchester, M (2009). Biomedical nanotechnology using virus-based nanoparticles. In Current Topics in Microbiology and Immunology (Steinmetz N. F. & Manchester M., eds) 327, 95-122.
Franssen, H., Goldbach, R., Broekhuijsen, M., Moerman, M. & van Kammen, A. (1982). Expression of middle-component RNA of cowpea mosaic virus: in vitro generation of a precursor to both capsid proteins by a bottom-component RNA-encoded protease from infected cells. Journal of Virology 41, 8-17.
Garcia, J. A., Schrijvers, I., Tan, A., Vos, P. Wellink, J. and Goldbach, R. (1987). Proteolytic activity of the cowpea mosaic virus encoded 24K protein synthesized in Escherichia coli. Virology 159, 67-75.
Goldbach, R. W. and Wellink, J. (1996). Comovirus: molecular biology and replication. In The Plant Viruses, vol. 5, pp. 35-76. Edited by B. D. Harrison & A. F. Murrant. New York: Plenum Press.
Holness, C. L., Lomonossoff, G. P., Evans, D. and Maule, A. J. (1989). Identification of the initiation codons for translation of cowpea mosaic virus middle component RNA using site-directed mutagenesis of an infectious cDNA clone. Virology, 172, 311-320.
Langeveld, J. P. M., Brennan, F. R., Martinez-Torrecuadrada, J. L., Jones, T. D., Boshuizen, R. S., Vela, C., Casal, J. I., Kamstrup, S., Dalsgaard, K., Meloen, R. H., Bendig., M. M. and Hamilton, W. D. O (2001). Inactivated recombinant plant virus protects dogs from a lethal challenge with canine parvovirus. Vaccine 19, 3661-3670.
Lin, T., Chen, Z., Usha, R., Stauffacher, C. V., Dai, J. B., Schmidt, T. & Johnson, J. E. (1999). The refined crystal structure of Cowpea mosaic virus at 2.8 \acute{Å} resolution. Virology 265, 20-34.
Liu, L. and Lomonossoff, G. P. (2002). Agroinfection as a rapid method for propagating Cowpea mosaic virus-based constructs. J Virol Meth 105, 343-348.
Liu, L., Cañzares, M. C., Monger, W., Perrin, Y., Tsakiris, E., Porta, C., Shariat, N., Nicholson, L. and Lomonossoff, G. P. (2005). Cowpea mosaic virus-based systems for the production of antigens and antibodies in plants. Vaccine 23, 1788-1792.
Lomonossoff, G. P. and Montague, N. P. (2008) Plant viruses as gene expression and silencing vectors. Encyclopedia of Life Sciences, John Wiley & Sons, Chichester. DOI 10.1002/9780470015902. a0020709.
Lomonossoff, G. P. & Shanks, M. (1983). The nucleotide sequence of cowpea mosaic virus B RNA. EMBO Journal 2, 2253-2258.
Lomonossoff, G. P. and Johnson J. E. (1991). The synthesis and structure of comovirus capsids. Prog. Biophys. Molec Biol 55, 107-137.
Nida, D. L., Anjos, R. J., Lomonossoff, G. P. & Ghabrial, S. A. (1992). Expression of cowpea mosaic virus coat protein precursor in transgenic tobacco plants. Journal of General Virology 73, 157-163.
Ochoa, W., Chatterji, A., Lin, T. and Johnson, J. E. (2006). Generation and structural analysis of reactive empty particles derived from an icosahedral virus. Chem. Biol. 13, 771-778.
Phelps, J. P., Dang, N. and Rasochova, L. (2007). Inactivation and purification of cowpea mosaic virus-like particles displaying peptide antigens from Bacillus anthracis. J. Virol. Meth. 141, 146-153.
Porta, C., Spall, V. E., Findlay, K. C., Gergerich R. C., Farrance, C. E. and Lomonossoff, G. P. (2003). Cowpea mosaic virus-based chimaeras. Effects of inserted peptides on the phenotype, host-range and transmissibility of the modified viruses. Virology 310, 50-63.
Rae, C., Koudelka, K. J., Destito, G., Estrada, M. N., Gonzales, M. J., Manchester, M. (2008) Chemical addressability of ultraviolet-inactivated viral nanoparticles (VNPs). PLoS ONE 3(10):e3315.doi:10.1371/journal.pone.0003315
Rezelman, G., van Kammen, A. and Wellink, J. (1989). Expression of cowpea mosaic virus M RNA in cowpea protoplasts. J. Gen. Virol. 70, 3043-3050.
Sainsbury, F. and Lomonossoff, G. P. (2008). Extremely high-level and rapid transient protein production in plants without the use of viral replication. Plant Physiology 148, 1212-1218.
Shanks, M. and Lomonossoff, G. P. (2000). Co-expression of the capsid proteins of cowpea mosaic virus in insect cells leads to the formation of virus-like particles. J Gen Virol 81, 3093-3097.
Steinmetz, N. F., Evans, D. J. and Lomonossoff, G. P. (2007). Chemical introduction of reactive thiols into a viral nanoscaffold: A method that avoids virus aggregation. Chem Bio Chem 8, 1131-1136.
Steinmetz, N. F., Lin, T., Lomonossoff, G. P. and Johnson, J. E. (2009). Structure-based Engineering of an Icosahedral Virus for Nanomedicine and Nanotechnology. In Current Topics in Microbiology and Immunology (Steinmetz N. F. & Manchester M., eds) 327, 23-58
Taylor, K. M., Spall, V. E., Butler, P. J. G. and Lomonossoff, G. P. (1999). The cleavable carboxyl-terminus of the small coat protein of cowpea mosaic virus is involved in RNA encapsidation. Virology 255, 129-137.
Van Bokhoven, H., Wellink, J., Usmany, M., Vlak, J. M., Goldbach, R. and van Kammen, A. (1990). Expression of plant virus genes in animal cells: high level synthesis of cowpea mosaic virus B-RNA-encoded proteins with baculovirus expression vectors. J. Gen. Virol. 71, 2509-2517.
Van Bokhoven, H., van Lent, J. W. M., Custers, R., Vlak, J. M., Wellink, J., and van Kammen, A. (1992). Synthesis of the complete 200K protein encoded by cowpea mosaic virus B-RNA in insect cells. J. Gen. Virol. 73, 2775-2784.
Van Kammen, A. (1971). Cowpea mosaic virus. CMI/AAB Descriptions of plant viruses No. 47.
Vézina, L-P., Faye, L., Lerouge, P., D'Aoust, M. A., Marquet-Blouin, E., Burel, C., Lavoie, P-O., Bardor, M. & Gomord, V. (2009). Transient co-expression for fast and high-yield production of antibodies with human-like N-glycans in plants. Plant Biotechnology Journal 7, 442-455
Vos, P., Verver, J., Jaegle, M., Wellink, J., van Kammen, A. and Goldbach, R. (1988). Two viral proteins involved in the proteolytic processing of the cowpea mosaic virus polyproteins. Nuc. Acids Res. 16, 1967-1985.
Wellink, J., Jaegle, M., Prinz, H., van Kammen, A. and Goldbach, R. (1987). Expression of middle component RNA of cowpea mosaic virus in vivo. J. Gen. Virol. 68, 2577-2585.
Wellink, J., Rezelman, G., Goldbach, R. And Beyreuther, K. (1986). Determination of the proteolytic processing sites in the polyprotein encoded by the bottom-component RNA of cowpea mosaic virus. Journal of Virology 59, 50-58.
Wellink, J., Verver, J., van Lent, J. and van Kammen. A. (1996). Capsid proteins of cowpea mosaic virus transiently expressed in protoplasts form virus-like particles. Virology 224, 352-355.
Wu, G.-J. & Bruening, G. (1971). Two proteins from cowpea mosaic virus. Virology 46, 506-512.
Young, M., Willits, D., Uchida, M. & Douglas, T, (2008). Plant Viruses as Biotemplates for materials and their use in nanotechnology. Annu. Rev. Phytopathol. 46, 361-384

1. A method of producing RNA virus capsids in a host cell, which method comprises:
(a) introducing one or more recombinant DNA vectors into the host cell or an ancestor thereof, wherein said one or more vectors comprise:
(i) a first nucleotide sequence encoding a polyprotein which can be proteolytically processed in the host cell to viral small (S) and lame (L) coat proteins from said RNA virus for assembly in the host cell into viral capsids; and
(ii) a second nucleotide sequence encoding a proteinase capable of said proteolytic processing;
(b) permitting expression of said polyprotein and proteinase from said first and second nucleotide sequences,
such that the polyprotein is proteolytically processed in the host cell to viral S and L coat proteins which assemble in the host cell into viral capsids,
which capsids are incapable of infection of the host cell.

2. A method as claimed in claim 1 wherein the one or more vectors are high-level expression vectors.

3. A method as claimed in claim 1 wherein the first nucleotide sequence encodes a polyprotein consisting essentially of the S and L coat proteins, one or both of which is optionally modified by way of sequence insertion, substitution, or deletion.

4. A method of producing RNA virus capsids in a plant cell, which method comprises:
(a) introducing one or more high-level expression recombinant DNA vectors into the plant cell or an ancestor thereof, wherein said one or more high-level expression recombinant DNA vectors comprise:
(i) a first nucleotide sequence encoding a viral S coat protein from said RNA virus; and
(ii) a second nucleotide sequence encoding a viral L coat protein from said RNA virus,
(b) permitting expression of said S coat protein and L coat protein from said first and second nucleotide sequences,
such that S and L coat proteins are assembled in the host cell into viral capsids,
and wherein the one or more vectors are high-expression vectors, which capsids are incapable of infection of the host cell.

5. A method as claimed in claim 4 wherein one or both of said S and L proteins is modified by way of sequence insertion, substitution or deletion.

6. A method as claimed in claim 3 wherein said modification is selected from the group consisting of: display of a heterologous peptide; incorporation of pores into the capsid; and incorporation of a tag to facilitate purification of the protein or capsid.

7. A method as claimed in claim 1 wherein the RNA virus capsids are essentially free of native viral genomic RNA.

8. A method as claimed in claim 7 wherein the RNA virus capsids are essentially free of RNA.

9. A method as claimed in claim 1 wherein the DNA vector or vectors do not encode entire native viral genomic RNA.

10. A method as claimed in claim 1 wherein the host cell is a plant cell, which is present in a plant.

11. A method as claimed in claim 10 wherein the DNA vector or vectors are plant vectors which include an expression cassette comprising:
(i) a promoter;
(ii) an enhancer sequence derived from the RNA-2 genome segment of a bipartite RNA virus, in which a target initiation site in the RNA-2 genome segment has been mutated;
(iii) said first and\or second nucleotide sequences;
(iv) a terminator sequence; and
(v) a 3′ UTR located upstream of said terminator sequence.

12. A method as claimed in claim 11 wherein the enhancer sequence consists of all or part of nucleotides 1 to 507 of the cowpea mosaic virus RNA-2 genome segment sequence shown in Table A, wherein the AUG at position 161 has been mutated as shown in Table B.

13. A method as claimed in claim 11 wherein said first nucleotide sequence encoding the polyprotein and said second nucleotide sequence encoding a proteinase are present on a single vector.

14. A method as claimed in claim 11 wherein the plant vector is a plant binary vector Which includes a suppressor of gene silencing.

15. A method as claimed in claim 10 further comprising harvesting a tissue from the plant in which the RNA virus capsids have been assembled, and isolating the capsids from the tissue.

16. A method as claimed in claim 15 wherein isolating the capsids from the tissue comprises the steps of:
(1) providing said plant tissue material;
(2) homogenising said material;
(3) adding an insoluble binding-agent which binds polysaccharides and phenolics;
(4) removing solid matter including said binding agent;
(5) precipitating the virus particles with a polyol;
(6) recovering the polyol precipitate, optionally by centrifugation;
(7) redissolving the pellet in aqueous buffer;
(8) high-speed centrifuging and discarding pelletable material not including said capsids;
(9) ultracentrifuging and discarding supernatant not including said capsids; and
(10) resuspending the pellet in aqueous buffer.

17. A method as claimed in claim 15 wherein isolating the capsids from the tissue does not comprise an organic solvent extraction step.

18. A method as claimed in claim 14 wherein the plant vector is a high-level expression vector such that % yield of isolated capsids from the harvested plant tissue is at least 0.01% or 0.02% w/w.

19. A method as claimed in claim 1 wherein the RNA virus is a bipartite RNA virus that is a member of the family Comoviridae.

20. A method as claimed in claim 19 wherein (i) the first nucleotide sequence encodes CPMV VP60 in which one or both of the CPMV S and L proteins is optionally modified by way of sequence insertion, subtitution or deletion; and (ii) the second nucleotide sequence encodes the CPMV 24K proteinase.

21. A method as claimed in claim 1 wherein the RNA virus capsids are subsequently chemically modified.

22. A gene expression system for producing RNA virus capsids in a host cell, which system comprises one or more high expression recombinant DNA vectors, wherein said one or more high expression recombinant DNA vectors comprise:
(i) a first nucleotide sequence encoding a polyprotein which can be proteolytically processed in the host cell to viral S and L coat proteins from said RNA virus for assembly in the host cell into capsids; and
(ii) a second nucleotide sequence encoding a proteinase from said RNA virus capable of said proteolytic processing.

23-24. (canceled)

25. A plant cell obtained or obtainable by a method of claim 10.

26. A plant which is selected from the group consisting of: a plant transiently transfected with a gene expression system of claim 22; and a transgenic plant stably transformed with a gene expression system of claim 22.

27. A method of producing RNA virus capsids encapsidating a desired payload in vitro, which method comprises:
(a) introducing a recombinant DNA vector into a host cell or an ancestor thereof, wherein said vector comprises a nucleotide sequence encoding a polyprotein which comprises viral small (S) and large (L) coat proteins from said RNA virus,
(b) permitting expression of said polyprotein from said nucleotide sequence, wherein said polyprotein is not proteolytically processed in the host cell to said viral S and L coat proteins,
(c) purifying said polyprotein from said host cell,
(d) contacting said polyprotein in vitro with (i) a proteinase capable of proteolytically processing the polyprotein to said viral S and L coat proteins and (ii) said payload,
such that the viral S and L coat proteins assemble in vitro into viral capsids encapsidating said payload.

28. A method as claimed in claim 27 wherein said polyprotein includes a tag at the N- or C terminal to facilitate protein purification.

29. An RNA virus capsid obtained or obtainable by a method of claim 1.

30. An RNA virus capsid as claimed in claim 29 which is a CPMV capsid essentially free of CPMV RNA.

31. An RNA virus capsid as claimed in claim 29 which is a CPMV capsid essentially free of CPMV RNA and which includes foreign protein sequence as part of the L or S sequence.

32. An RNA virus capsid as claimed in claim 31 wherein the foreign protein sequence is a tag at the N- or C terminal to facilitate protein or capsid purification.
